(19) United States
(12) Patent Application Publication
(10) Pub. No.: US 2026/0102359 A1
(43) Pub. Date: Apr. 16, 2026

(54) COMBINATION TREATMENT OF DERMAL AND TRANSDERMAL FIBROTIC DISEASES, DISORDERS AND ASSOCIATED PAIN AND INFLAMMATION

(71) Applicant: ERESINA, LLC, Aliso Viejo, CA (US)

(72) Inventors: David SCHARP, Mission Viejo, CA (US); Mukhtar SIDDIQUI, San Ramon, CA (US); Jennifer HURTIKANT, Downey, CA (US); David BONDURANT, San Clemente, CA (US); Brian HAIGHT, Yorba Linda, CA (US); Kai HANSEN, Venice, CA (US); David J. AUSTIN, Aliso Viejo, CA (US)

(21) Appl. No.: 19/114,930

(22) PCT Filed: Sep. 28, 2023
(86) PCT No.: PCT/US2023/034058

§ 371 Date: Mar. 25, 2025

Related U.S. Application Data
(60) Provisional application No. 63/411,052, filed on Sep. 28, 2022.

(51) Int. Cl. A61K 31/015; A61K 9/00; A61K 31/282; A61K 36/886; A61K 38/48; A61K 45/06; A61P 1/04; A61P 17/02; A61P 17/14; A61P 19/00; A61P 31/10

(52) U.S. Cl. CPC A61K 31/015; A61K 9/0014; A61K 31/282; A61K 36/886; A61K 38/4886; A61K 45/06; A61P 1/04; A61P 17/02; A61P 17/14; A61P 19/00; A61P 31/10; C12Y 304/24003

(57) ABSTRACT

The present disclosure is directed to topical compositions and methods for the treatment and/or amelioration of dermal and transdermal fibrotic diseases, disorders and associated pain and inflammation, including Dupuytren's disease, Dupuytren's contracture. Ledderhose's disease, Peyronie's disease, scleroderma, and Raynaud's (or Renaud's) phenomenon in combination with existing treatments of these disorders.

### CROSS-REFERENCE TO RELATED APPLICATIONS

This non-provisional application claims the benefit of U.S. Application Ser. No. 63/411,052, filed on Sep. 28, 2022, which claims the benefit of U.S. application Ser. No. 16/882,429, filed on May 22, 2020, which claims the benefit of U.S. application Ser. No. 16/198,793, filed on Nov. 22, 2018, which claims the benefit of U.S. Provisional Application No. 62/590,251, which was filed on Nov. 22, 2017. Each of these applications is incorporated by reference herein in its entirety.

### FIELD OF THIS DISCLOSURE

The compositions and methods of the present disclosure relate generally to the field of combination treatments for fibrotic diseases, disorders and associated pain and inflammation, and methods of manufacture thereof. The application further discloses compositions for treatment of topical orphan diseases and related syndromes, and methods of treatment and process of manufacture of disclosed compositions.

### BACKGROUND OF THE INVENTION

Numerous diseases and disorders are associated with excess collagen deposition and the erratic accumulation of fibrous tissue rich in collagen. Such diseases and disorders are often referred to as fibrotic diseases and disorders. These include but are not limited to Dupuytren's disease (palmar fascia fibromatosis), Dupuytren's contracture, Ledderhose's disease, Peyronie's disease, scleroderma, and Raynaud's (or Renaud's) phenomenon, wound healing, such as skin damage from cuts, abrasions and burns, as well as aging skin changes, toe nail fungus, and chemotherapy and/or radiation induced oral mucosal ulceration and aphthous ulcers.

Orphan diseases include several well-known diseases, like cystic fibrosis and Tourette's syndrome, but most are unknown to the general public. The orphan diseases of interest in this application are Dupuytren's Contracture, Peyronie's Disease, Scleroderma, Raynaud's (or Renaud's) Phenomenon, Chemotherapy/Radiation Induced Oral Mucosal Ulceration, and Aphthous Ulcers; and more frequent skin issues of Skin Damage from Cuts, Abrasions, and Burns; Aging Skin Changes, and Toe Nail Fungus.

Treatments for orphan diseases such as these pose problems not associated with more frequent diseases. First, since few people are inflected, there is little awareness of their problems, so few treatments are investigated. Additionally the low frequency of occurrence hinders the development of these treatments due to the feeling that it will be difficult for a company to recover the high costs of development. An orphan disease is defined as a condition that affects fewer than 200,000 people nationwide. Collectively, however, they affect as many as 25 million Americans, according to the National Institutes of Health (NIH), and that makes the diseases—and finding treatments for them—a serious public health concern.

Patients diagnosed with a rare disease are often denied access to effective medicines because prescription drug manufacturers rarely could make a profit from marketing drugs to such small groups. Consequently, the prescription drug industry has not adequately funded research for orphan product development. Despite the urgent health need for these medicines, they came to be known as orphans because companies were not interested in adopting them.

The diseases and disorders of the present invention include diseases and disorders having a fibrotic component. Fibrosis is a pathogenic hallmark of a vast number of conditions, implicating a wide variety of tissues, including joints and interstitial tissues (e.g., arthrofibrosis, Dupuytren's disease), the skin (e.g., keloid lesions, nephrogenic systemic fibrosis, scleroderma), the mouth (e.g., fibrous proliferative lesions of the oral cavity), the vasculature, including the peripheral vasculature, among others. Fibrosis may result from environmental insults or injury, such as, for example, exposure to ionizing radiation (such as during cancer treatments), as a result of cystic rupture in the breast, (causing palpable lesions in mammary tissue), and generally as a result of overdeposition of collagen following a wound or tissue insult, such as after injury or surgery.

While some types of fibrosis involve underlying genetic predispositions (e.g., Dupuytren's disease), many types of fibrosis involve prolonged inflammation of the affected tissue. Symptoms may be as minor as pruritis and aesthetic concerns, such as keloid lesions of the skin, or as significant as pulmonary failure and death, for example in terminal symptoms of pulmonary fibroses and cardiac fibroses. Current antifibrotic treatments include anti-inflammatory compounds such as pirfenidone and fibroblast growth factor receptor antagonist nintedanib. For dermal and subdermal fibroses, therapies are largely limited to surgery, phototherapy and/or injections at the site of fibrosis. Due to the invasiveness and limited efficacy of current therapies, there remains a need for additional therapeutic approaches to this class of conditions.

Inflammation is part of the complex biological response of body tissues to harmful stimuli, such as pathogens, damaged cells, or irritants, and is a protective response involving immune cells, blood vessels, and molecular mediators. The function of inflammation is to eliminate the initial cause of cell injury, clear out necrotic cells and tissues damaged from the original insult and the inflammatory process, and initiate tissue repair. Inflammation can be classified as either acute or chronic. Acute inflammation is the initial response of the body to harmful stimuli and is achieved by the increased movement of plasma and leukocytes from the blood into the injured tissues. A series of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation, such as mononuclear cells, and is characterized by simultaneous destruction and healing of the tissue from the inflammatory process, which often leads to a host of diseases including fibroses.

Because inflammation often occurs in connection with fibroses, fibrotic diseases, disorders and conditions, and within and around fibrotic systems, there is a need for therapeutic approaches that can treat both fibroses and inflammation.

Dupuytren's disease (also known as (palmar fascia fibromatosis)) is a disease associated with the buildup of extracellular matrix materials such as collagen in the connective tissue characterized by thickening of the palmar fascia of the hand. The connective tissue thickens and shortens with the physical effect of causing the fingers to curl, most commonly the ring finger and little finger. Dupuytren's disease is a complex fibrosis proliferative disorder of the hand that is often progressive and eventually can cause contractures of the affected fingers. Contracture is referred to as Dupuytren's contracture and is an advanced stage of the disorder that is particularly difficult to treat.

Dupuytren's disease is characterized by thickening and contracture of the fascia (connective tissue) of the palm, usually progressing to flexion deformities and involvement of one or more fingers. This results from formation of longitudinal cords of indurated fibrous tissue in the palm and extending into the finger. A similar lesion sometimes occurs in the foot, known as Ledderhose's disease.

Dupuytren's disease affects approximately 5% of the white Caucasian population. The most common manifestation is progressive flexion contracture of the fingers, resulting in significantly compromised function. The causes of Dupuytren's disease are not well understood and underlying disease is not currently curable.

Dupuytren Contracture is one of the diseases with few people but who suffer great pain and difficulty with life. About 15 million Americans age 35 and older have Dupuytren disease, which is about 5% of the US population. 3 million in the US have bent fingers from Dupuytren disease and 750,000 Americans have severe Dupuytren biology and the risk of being crippled even with available treatment. After age 55, the percent of people with Dupuytren disease increases steadily in both men and women. By age 70, one-quarter of men have signs of Dupuytren, increasing to one-third by age 80. Dupuytren is more common in women than men in those 85 and older. The total number of US Dupuytren cases increased from 14.2 million in 2000 to 16.2 million people in 2010. The percent of the US population 35 and older affected with Dupuytren disease increased from 8.7% in 2000 to 9.3% in 2010.

Dupuytren's Contracture is a rare connective tissue disorder characterized by fixation of the joints of certain fingers in a permanently flexed position. Due to abnormal thickening and shortening of the bands of fibrous tissue beneath the skin of the palm, a hardened nodule may develop, eventually forming an abnormal band of hardened fibrotic tissue. This causes the fingers of the affected area to begin to be drawn in toward the palm over several months or years and cannot be pulled back. The skin of the affected area may pucker. The ring and pinky fingers are most affected and usually both hands are affected.

It has been demonstrated in the scientific literature that the nodules formed in Dupuytren's disease, and the tissue of fibrotic diseases in general, largely comprise myofibroblast cells. (See, e.g., Verjee L S, Verhoekx J S, Chan J K, Krausgruber T, Nicolaidou V, Izadi D, Davidson D, Feldmann M, Midwood K S, Nanchahal J. Unraveling the signaling pathways promoting fibrosis in Dupuytren's disease reveals TNF as a therapeutic target. Proc Natl Acad Sci USA. 2013 Mar. 5; 110(10):E928-37 at pg. E929, right column, lines 5-9.) Verjee, et al. disclose that 87%±6% of the cells isolated from a Dupuytren's nodule were myofibroblasts. (Id.)

Myofibroblast cells are known to be involved in both natural fibrosis and fibrotic disorders throughout the human body. (See, e.g., Hinz B, Lagares D. Evasion of apoptosis by myofibroblasts: a hallmark of fibrotic diseases. Nat Rev Rheumatol. 2020 January; 16(1): 11-31.) Inhibition of myofibroblasts has been reported to be a viable therapeutic target for the treatment and potential reversal of fibrosis disorders. (Id. at pg. 23, right column, lines 15-17.)

The present invention involves, in part, mitigation of myofibroblasts through inhibition of crucial myofibroblast signalling pathways and cytotoxicity. The present invention further involves mitigation of the inflammation associated with fibrosis and fibrotic disorders by mitigating the inflammatory response, including inhibition of crucial inflammatory cell signalling pathways and cytotoxicity, for example macrophages.

Treatment of Dupuytren's disease and contracture has traditionally involved surgical excision of the offending tissue. In severe or recurrent disease, the surgical excision may be combined with excision of the overlying palmar skin and resurfacing of the cutaneous defect with full thickness skin graft. Surgery is typically followed by prolonged rehabilitation, usually lasting 3 months and complications have been reported in up to 20% of cases. Such surgical correction is the mainstay treatment of later stage Dupuytren's contracture where secondary changes to tendons and joints have developed. A less invasive surgical intervention is needle aponeurectomy in which the fibrous bands (contractures) in connective tissue are divided using the bevel of a needle.

Enzymatic cleavage of the affected tissue has been the focus of development to reduce invasiveness associated with surgery and improve recovery time of surgical treatment. Clostridial collagenase, a bacterial collagenase, has been granted FDA approval as Xiaflex®. U.S. Pat. No. RE39,941, U.S. Pat. Nos. 5,589,171 and 6,086,872 describe the use of bacterial collagenase for the enzymatic cleavage of connective tissue in the treatment of Dupuytren's disease. Xiaflex® is associated with high cost, possible allergic reactions, development of neutralizing antibodies and a high recurrence rate. Bacterial collagenase therapy has been associated with improved outcomes in the treatment of Dupuytren's disease when injected into the affected areas of contractures, but it is expensive and is associated with development of neutralizing antibodies. The combination of these therapies with the topical compositions of the present invention overcome these limitations.

Peyronie's disease is a connective tissue disorder of the penis that may be likened to Dupuytren's contracture of the hand. It is characterized by the triad of bent erections, pain in the penis with erections and palpable penile plaque. Peyronie's disease is quite common, affecting as many as one in 11 men, despite the lack of public awareness. The penis is composed of the same connective tissue as every other joint in the body. The anatomy of the penis is composed of three cylinders: the paired erectile bodies and the urethra. The erectile bodies (corpora cavernosa) are made up of sinusoidal tissue that fills up with blood during an erection and an outer covering (tunica albuginea) composed of tough fibroelastic tissue. The outer covering determines the size and shape of the erection.

The principal finding in men with Peyronie's disease is the deposition of scar tissue in the tunica albuginea. All of the clinical symptoms are derived from this event. The curvature of the penis is due to the fact that scar tissue does not stretch as well as normal tissue. The normal tunica albuginea is composed of elastin fibers and collagen. The site of scar tissue from Peyronie's disease is composed mostly of collagen that may harden to the thickness of bone.

Although most men with Peyronie's disease report that their penis is bent upwards, a variety of other abnormalities are frequently observed, including bends in other directions, complex bends, divots in the side of the penis and hourglass deformities. The palpable plaque is the actual scar tissue that has been deposited on the outer covering of the erectile bodies. This is present in the vast majority—but not all—patients with Peyronie's disease. The plaque may become calcified, like bone, with severe disease. The pain experienced with erections in men suffering from Peyronie's disease is believed to be due to active inflammation in the plaque.

The exact etiology of Peyronie's disease is not currently known. There is evidence of an association with other connective tissue disorders, specifically Dupuytren's contracture, affecting the palms of the hands. The most prevalent current theory is that Peyronie's disease is induced by trauma. The trauma may be acute and distinct such as a penile fracture, but more often it is chronic and low grade, such as repeated attempts at sexual intercourse with weak or incomplete erections.

Peyronie's disease is similar to Dupuytren's Contracture as both involve a connective tissue disorder. Peyronie's disease is characterized by the development of fibrous plaques in the soft tissue of the penis of adult males which results in penile deformity. It is estimated to affect 0.5% adult males in the United States, although it is difficult to know the actual extent due to the hesitancy of disclosing the problem. Affected individuals may experience pain, have cord-like lesions on the penis, and exhibit abnormal curvature of the penis when erect. The abnormal curvature of the penis may make it impossible for affected individuals to have normal sexual intercourse.

Current medical therapies for Peyronie's disease are lacking. Surgical therapy (penile straightening) is employed when there is a significant penile defect preventing sexual relations and is often limited to more serious afflictions. Potential complications of penile-straightening surgery are palpable suture knots, penile shortening (approximately 1 centimeter for every 15 degrees correction), numbness of the penis, residual curvature and worsening erectile dysfunction. As such, there is a great need for a non-surgical, topical treatment for men suffering from Peyronie's disease.

Scleroderma is a rare, chronic disease of the immune system, blood vessels and connective tissue affecting about 2.5 million people worldwide and an estimated 300,000 people in the United States. The disease is three to four times more common in females than in males and may occur at any age but most frequently the symptoms begin in midlife. Scleroderma is an autoimmune connective tissue disorder characterized by abnormal thickening of the skin. Connective tissue is composed of collagen, which supports and binds other body tissues. Some types of sclerodermas affect certain, specific parts of the body, while other types can affect the whole body and internal organs. The exact cause of scleroderma is unknown and because the symptoms are similar to those of other autoimmune diseases, therefore diagnosis is difficult.

The early symptoms of scleroderma vary considerably but distinctive abnormalities on the skin usually appear later in the course of the disease. Common symptoms include painful joints, morning stiffness, fatigue, and weight loss. People with scleroderma have areas of skin that become hard and leathery. These areas of hardness are widespread and typically appear on both sides of the body. Eventually, tissue loss occurs, and the skin becomes more highly colored. The skin can become thin, shiny, and bright which results in decreased function of the fingers and toes. Treatment of scleroderma is typically limited to symptomatic and supportive care. Medications can be administered to ameliorate the hardening of the skin, although to date they have not proven to be very effective. Skin care may include lubricating creams or antibiotic ointments for infected ulcerations.

Raynaud's (or Renaud's) phenomenon may be associated with scleroderma, although the disorder can occur in subjects without scleroderma. Raynaud's phenomenon is a vascular disorder characterized by the intermittent loss of blood to various parts of the body, particularly the fingers, toes, nose, and ears causing them to turn white. Symptoms typically occur after exposure to cold, causing tingling sensations, numbness, and pain. Raynaud's phenomenon is often an early complaint of subjects having scleroderma.

An estimated 28 million people in the United States have Raynaud's phenomenon. The major symptom is a dramatic stark white pallor of affected areas when exposed to cold, although a blue or red color may also be present from time to time. Other symptoms include a feeling of numbness, severe aching or pain, tingling or throbbing, a sensation of tightness, “pins and needles,” and a profound loss of sensation. Drug therapy with vasodilators may be used to assist widening of the blood vessels, but no direct treatment is currently available.

Collagen is a major structural component of mammalian organisms and makes up a large portion of the total protein content of skin and other parts of the mammalian body. In humans, collagen is important in the wound healing process and the process of natural aging. Various skin traumas such as burns, surgery, infection and accident are often characterized by the erratic accumulation of fibrous tissue rich in collagen and having increased proteoglycan content. In addition to the replacement of the normal tissue which has been damaged or destroyed, excessive and disfiguring deposits of new tissue sometimes form during the healing process. The excess collagen deposition has been attributed to a disturbance in the balance between collagen synthesis and collagen degradation.

Fibrotic processes involved in wound healing may include topical injuries or surgical procedures. Typical topical injuries include abrasions, acute sun burns, cat scratches, chemical burns, dog bites, finger/toe nail damage, heated object burns, spider and insect bites and superficial cuts. Other topical disorders include cutaneous rashes due to allergic reactions, poison ivy, poison oak, secondary infectious rashes, including measles and chicken pox. Wounds due to surgical procedures include all wounds made in and through the skin, emergent first aid, regardless of type of surgery, skin grafts, and sutured and non-sutured incisions. Other disorders contemplated by the present invention include aged skin, sun exposed skin surfaces, and baldness.

Another condition that causes severe pain and difficulty is oral mucosal ulceration induced by chemotherapy and/or radiation therapy. Although not a disease itself, oral mucosal ulceration is a common disorder for patients undergoing cancer treatment. Radiation-induced oral mucosal ulceration occurs in up to 80% of head and neck cancer irradiated patients and reaches up to 100% of patients with altered fractionation head and neck cancer.

Radiation-induced oral mucosal ulceration is a major dose-limiting toxicity in head and neck cancer patients. It is similar to a normal tissue injury caused by radiation, which has marked adverse effects on patient quality of life and cancer therapy continuity. It remains a challenge for proper cancer treatment because it leads to therapy interruption, poor local tumor control, and changes in dose fractionation. The economic cost of oral mucosal ulceration in cancer treatment is estimated to be tens of thousands of dollars per patient being treated for head and neck cancers.

Aphthous Ulcers (also called canker sores) are similar to the oral mucosal ulceration caused by cancer treatments but occur in individuals with no known cause. Aphthous ulcers are small, shallow lesions that develop on the soft tissues in the mouth or at the base of the gums. Aphthous ulcers do not occur on the surface of the lips and are not contagious. However, they are painful and make eating and drinking uncomfortable and difficult. Most ulcers disappear on their own within one to two weeks, but some may last longer. An effective treatment for these ulcers would greatly benefit the quality of life for those who suffer from them with some frequency.

The aphthous ulcers are round or oval with a white or yellow center and a red border. The ulcers form inside the mouth; on or under the tongue, inside the cheeks or lips, at the base of the gums, or on the soft palate. Appearance is often accompanied by a tingling or burning sensation a day or two before the sores appear.

Numerous treatments for fibrosis/collagen-related diseases and disorders involve invasive procedures such as surgery or multiple injections into the affected tissue. Therefore, a topical treatment for fibrosis/collagen-related diseases and disorders highly advantageous to those suffering from the symptoms of these diseases.

Another problem with treating these orphan diseases was the inability to furnish compositions with sufficient concentrations of active ingredients. Additionally, some of the active ingredients were hydrophilic and other active ingredients were hydrophobic. It is difficult to apply hydrophilic and hydrophobic ingredients at the same time in sufficient concentrations as to be effective against the disease.

Previously, the hydrophilic and hydrophobic ingredients would separate before application, which prevented their combined use. The investigator of this application was able to solve this formulation problem that had plagued other researchers. Additionally, the disclosed process of manufacture presented unexpected results in both ingredient concentration and compatibility.

Additionally, it was discovered during the development of the disclosed compositions and treatments that the compositions were additionally effective against more common skin and oral problems. Variations of the compositions for the orphan diseases were able to treat skin damage from cuts, abrasions, and burns; aging skin changes; and toe nail fungus. This discovery increased the population who could be helped by these compositions.

The disclosed compositions and methods relate to treating medical conditions that are known to have few, if any, effective treatments currently available. There are many medical conditions that exist today without currently demonstrated effective medical treatments that are the subject of extensive research by major pharmaceutical companies throughout the world. The goal is to develop potentially beneficial, skin care compositions that demonstrate new effectiveness following composition component alteration and manufacturing in multiple medical conditions without known and effective treatments.

It has surprisingly and unexpectedly been found that topically applied compositions comprising α-pinene and/or aloe vera inner leaf gel, in combination with one or more adjuvants disclosed herein, and in combination with known therapies, have been found to alleviate the severe symptoms of fibrosis/collagen-related diseases and disorders.

### SUMMARY OF THIS DISCLOSURE

The present invention includes compositions and combination methods for the treatments of dermal and transdermal fibrotic diseases, disorders and associated pain and inflammation, and methods of manufacture thereof. The present invention further includes compositions for the combination treatment of dermal and transdermal orphan diseases and related syndromes, and methods of treatment and process of manufacture of disclosed compositions.

### DETAILED DESCRIPTION OF THIS DISCLOSURE

The following description sets forth exemplary methods, parameters and the like of the present invention. It should be recognized, however, that such description is not intended as a limitation on the scope of the present disclosure but is instead provided as a description of exemplary embodiments. Any terms used throughout this disclosure have their plain and ordinary meaning in the art. Any definitions herein are provided to disclose certain specific uses of those terms, unless specifically indicated otherwise. The plural and singular should be treated as interchangeable, other than the indication of number.

The present disclosure provides in certain embodiments novel pharmaceutical and medicinal formulations for the treatment of fibrosis/collagen-related diseases and disorders and associated pain and inflammation.

In one embodiment, the compositions of the present disclosure may be administered to treat, ameliorate, prevent, or cure one or more fibrotic diseases or disorder, including, Dupuytren's disease, Dupuytren's contracture, Ledderhose's disease, Peyronie's disease, scleroderma, and Raynaud's (or Renaud's) phenomenon, wound healing, such as skin damage from cuts, abrasions and burns, as well as aging skin changes, toe nail fungus, and chemotherapy and/or radiation induced oral mucosal ulceration, aphthous ulcers, and other diseases or conditions resulting in the excessive deposition of extracellular matrix components, such as collagen.

Another embodiment of the present invention is a method of treatment and/or alleviation of the pain and inflammation associated with the fibrotic diseases and disorders disclosed herein.

The methods and compositions according to the present disclosure may also comprise a primary fibrosis, or a condition in which said fibrosis, fibrotic condition or fibrotic symptom is secondary to or symptomatic of another condition.

One embodiment of the present invention is a combination therapy of the compositions of the present invention in combination with known compositions, methods and modalities for the treatment of dermal and transdermal fibrotic diseases and disorders, including but not limited to adalimumab (Humira®), fondaparinux (Arixtra®), Enoxaparin, Bivaliruden, a factor Xa inhibitor, a collagenase (e.g., Xiaflex®, bacterial), and endopeptidase.

As used herein, the term “combination” includes the administration of the compositions and/or agents being administered prior to, concurrent with, or subsequent to each other to the patient.

In particular certain embodiments comprise comprising α-pinene, aloe vera inner leaf gel, shea butter, methyl sulfonyl methane, black cumin seed oil, collagen, lecithin, glycerol, in combination with one or more adjuvants, for the treatment of the fibrosis/collagen-related diseases and disorders of the present disclosure.

In one embodiment, a composition comprising α-pinene is provided.

In one embodiment, a composition comprising aloe vera is provided. In another embodiment, a composition comprising aloe vera inner leaf gel is provided.

In one embodiment, a composition comprising shea butter is provided. In another embodiment, a composition comprising Ghana shea butter is provided.

In one embodiment, a composition comprising methyl sulfonyl methane (MSM) is provided.

In one embodiment, a composition comprising black cumin seed oil is provided.

In one embodiment, a composition comprising collagen is provided.

In one embodiment, a composition comprising lecithin is provided.

In one embodiment, a composition comprising glycerol is provided.

In one embodiment, the composition of the present disclosure comprises one or more adjuvants, which are comprised in the formulation, act as a vehicle, carrier, pharmaceutical carrier and/or increase the efficacy and/or potency of the composition.

In another embodiment, a composition comprising one or more adjuvants selected from(s)-(−)-perillic acid, 12-hydroxydodecanoic acid, 2-hydroxy benzoate, 3-hydroxyacetophenone, 3-hydroxybenzoic acid, 4(4′-hydroxyphenyl)-2-butanone, 4,6-dimethyl-1-heptanol, 4-ethyiphenol, 4-methoxyacetophenone, 4-methylcatechol, 5,7-dihydroxyflavanone, 5-aminosalicylic acid, 5-methoxylsalicylic acid, 6-methyl-1-heptanol, acetophenone, acetyl salicylic acid, additional: ajoene, alipinetin, α-lipoic acid, allicin, allopurinol, allopurinol sodium, alpha tocopherol (vitamin E), alpinetin, amidoamine, apigenin, arachidonic acid, asarinin (desmin), ascorbic acid vit c, ascorbyl palmitate, α-tocopheryl acetate VTE, baicalein, baicalein 7-O-β-D-glucuronide, baicalin, bemethyl, benzoic acid, benzyl alcohol, bilirubin, borneol, butylated hydroxytoluene, butyric acid, caffeic acid, caffeic acid, calcium ascorbate, carvacrol, castoramine, catechol, chlorovanillin, chromanol, Chrysanthemin, cianidanol (catechin), cyanidin, cyanidin-3-glucoside, cyclohexane-1,2,-diol, dihydroferulic acid, dihydrolipoic acid, dihydrolipoic acid, dimethyl isosorbide, dimethyl sulfoxide (DMSO), dipyridamole (persantin), epicatechin, erythorbic acid, eugenol, ferulic acid, gastrodin, genipin, glyceollin i, glycylglyciine (diglycine), halofuginone, hesperidin (glycoside #106), hesperitin, hesperitin, hydroquinone, hydroxytyrosol, icariin, iso-ascorbic acid. (dl), isopinocamphone, kaempferol, kynurenic acid, lauric acid, lecithins (hydrogenated), linalool alkaloid, linalool oxide a, lithospermic acid, lithospermic acid b, luteolin, lycopene, lycopine, melatonin, menthol, mnt4mpyp, mofebutazone, morelloflavone, morin, morin hydrate, murrayafoline a, naringenin, naringin, nupharamine, obovatal, o-creosol, pentoxifylline, phenol, pinocamphone, pinocembrin, pinocembrin, pinocembrin, polydatin, Reservatrol, probucol (Lorelco), propanediol, propylene glycol, puerarin, quercitin, quercitin, rel-alpha vitamin e, rhein, rosmarinate, S.R.R. alpha-tocopherol, salicylaldehyde, salicylamide, salicylate, salicylic acid, salsalate, salvianolic acid a, salvianolic acid a, salvianolic acid b, salvianolic acid b, sesamol, shogaol, silibinin (silybin), silymarin, sodium ascorbate, sodium erythorbate, sodium ferulate, terephthalic acid, tetrahydro-curcumin, theobromine, theophylline, tocopherol acetate vit e, tocotrienol, Trimidox, Trolox, uric acid, vanillin, verbascoside, violuric acid, vitamin e, vitamin e acetate is provided.

In another embodiment, a composition comprising one or more adjuvants selected from 4-(nicotinamido)butanic acid, 5,7-dihydroxyflavanone, allicin, allopurinol, allopurinol sodium, alpha tocopherol, ammonium purrolidyldithiocarbamate, anisodamine, arachidonic acid, asarinin (desmin), ascorbic acid (vitamin C), ascorbyl palmitate, bemethyl, beta-carotene, bilirubin, bromovanillin, butylated hydroxytoluene, butyric acid, caffeic acid, calcium ascorbate, carvacrol, chlorovanillin, Chromal, chromanol, cianidanol (catechin), cyanidin, cyanidin-3-glucose, diallyl sulfide, dihydroferulic acid, dihydrolipoic acid, dimethyl sulfoxide (DMSO), DL-alpha-tocopherol acetate, epicatechin, erythorbic acid, eugenol, ferulic acid, glutathione, glycylglycine, hydroxxytyrosol, iso-ascorbic acid, kaempferol, lecithin (hydrogenated), α-lipoic acid, lithospermate b, lithospermic acid, lithospermic acid b, luteolin, lycopene, manganese-platinum organic complexes (e.g., MntP-bio2 and Mnt4MPyP), melatonin, morin, morin hydrate, nicareven, pegorgotein, pentoxifyline, phytic acid, pinocembrin, proamipider, probucol, probucolc (Lorelcoti), propyl gallate, puerarin, quercitin, rebamipide, rel-alpha (vitamin E), reservatrol, retinol, rosemarinate, rosmarinic acid, salvianolic acid a, sesamol, silibyn (silininin), silymarin, sodium ascorbate, sodium erythrobate, sodium ferulate, sodium terephthalate, S.R.R alpha-tocopherol, terephthalic acid, tetrahydro-curcumin, thicotic acid, tocopheryl acetate, trans-caffeic acid, Trimidox (vetoquinol), Trocotrienl, Trolox, Tyrosol, ubiquinol-10, ubiquinone, uric acid, vanillin, verbascoside, violuric acid, vitamin A, vitamin E, vitamin E acetate.

In another embodiment, a composition comprising one or more adjuvants selected from 6-shagaol, ajoene, baicalin, gastrodin, genipin, halofuginone, kynurenic acid, lycopene, mofebutazone, obavatol, perillic acid, rhein is provided.

In another embodiment, a composition comprising one or more adjuvants selected from alipinetin, bicalin, glyceollin 1, hesperidin glycoside, hesperitin, icariin, morelloflavon, murrayafoline A, naringenin, naringin, pinocembrin, puerarin, quercitin is provided.

In another embodiment, a composition comprising one or more adjuvants selected from 3-hydroxyacetophenone, 3-hydroxybenzoic acid, 4(4′-hydroxyphenyl)-2-butanone, 4,6-dimethyl-1-heptanol, 4-ethyiphenol, 4-methoxyacetophenone, 4-methylcatechol, 5-methoxylsalicylic acid, 6-methyl-1-heptanol, acetophenone, benzoic acid, benzyl alcohol, borneol, castoramine, catechol, cyclohexane-1,2,-diol, hydroquinone, isopinocamphone, linalool alkaloid, linalool oxide a, nupharamine, o-creosol, phenol, pinocamphone, salicylaldehyde is provided.

In another embodiment, a composition comprising one or more adjuvants selected from 3-hydroxyacetophenone, 3-hydroxybenzoic acid, 4,6-dimethyl-1-heptanol, 4-ethyiphenol, 4-methoxyacetophenone, 4-methylcatechol, 5-methoxylsalicylic acid, 6-methyl-1-heptanol, acetophenone, catechol, isopinocamphone, linalool alkaloid, linalool oxide a, pinocamphone, salicylaldehyde is provided.

In another embodiment, a composition comprising one or more adjuvants selected from 3-hydroxyacetophenone, 3-hydroxybenzoic acid, 4-ethyiphenol, 4-methoxyacetophenone, 4-methylcatechol, 5-methoxylsalicylic acid, acetophenone, catechol, salicylaldehyde is provided.

In another embodiment, a composition comprising one or more adjuvants selected from 3-hydroxyacetophenone, 4-ethyiphenol, acetophenone, catechol is provided.

In one embodiment, a composition comprising 4-(nicotinamido)butanic acid is provided.

In one embodiment, a composition comprising alpha tocopherol is provided.

In one embodiment, a composition comprising ammonium purrolidyldithiocarbamate is provided.

In one embodiment, a composition comprising asarininn is provided.

In one embodiment, a composition comprising bilirubin is provided.

In one embodiment, a composition comprising bromovanillin is provided.

In one embodiment, a composition comprising butylated hydroxytoluene is provided.

In one embodiment, a composition comprising caffeic acid is provided.

In one embodiment, a composition comprising chlorovanillin is provided.

In one embodiment, a composition comprising Chromal is provided.

In one embodiment, a composition comprising diallyl sulfide is provided.

In one embodiment, a composition comprising dihydrolipoic acid is provided.

In one embodiment, a composition comprising dimethyl sulfoxide is provided.

In one embodiment, a composition comprising DL-alpha-tocopherol acetate is provided.

In one embodiment, a composition comprising feruic acid is provided.

In one embodiment, a composition comprising hydroxytyrosol is provided.

In one embodiment, a composition comprising lithospermate B is provided.

In one embodiment, a composition comprising lycopene is provided.

In one embodiment, a composition comprising manganese-platinum organic complexes (e.g., MNTP-Bio2 and MNT4MPYP) is provided.

In one embodiment, a composition comprising melatonin is provided.

In one embodiment, a composition comprising nicareven is provided.

In one embodiment, a composition comprising proamipider is provided.

In one embodiment, a composition comprising probucol is provided.

In one embodiment, a composition comprising rebamipide is provided.

In one embodiment, a composition comprising reservatrol is provided.

In one embodiment, a composition comprising rosmarinic acid is provided.

In one embodiment, a composition comprising Sesamol is provided.

In one embodiment, a composition comprising Silibyn (silininin) is provided.

In one embodiment, a composition comprising sodium terephthalate is provided.

In one embodiment, a composition comprising terephthallc acid is provided.

In one embodiment, a composition comprising thicotic acid is provided.

In one embodiment, a composition comprising trans-caffeic acid is provided.

In one embodiment, a composition comprising Trimidox (vetoquinol) is provided.

In one embodiment, a composition comprising Trolox is provided.

In one embodiment, a composition comprising Tyrosol is provided.

In one embodiment, a composition comprising uric acid is provided.

In one embodiment, a composition comprising vanillin is provided.

In one embodiment, a composition comprising violuric acid is provided.

In one embodiment, a composition comprising vitamin E is provided.

In one embodiment, a composition comprising vitamin E acetate is provided.

In another embodiment, a composition comprising one or more adjuvants selected from 1,2-hexanediol, 1,2-octanediol (caprylyl glycol), 1-tetradecanol, a phycobiliprotein, acai berry oil, agave nectar, ajwain oil, alanine, almond oil, Aloe vera inner leaf gel, alpha-cadinol, anethole oil, anise seed oil, annato, apricot kernal oil, arachidonic acid, arctic fish (AF) collagen, argan nut oil, arginine, atlas cedar wood oil, baobab oil, bayberry wax, bees wax, benzoic acid, benzoin, benzyl alcohol, berberine, betaine, bixin, black cumin seed oil, Boesenbergia rotunda, cajuput or cajeput oil, calendula oil, camellia oil, canolol oil, capric (decanoic) acid, caprylic, capric triglyceride, caramel powder, carnauba wax, carvacrol, caryophyllene, cassia oil, cedar oil, cetyl alcohol, Chamomile German oil, chlorogenic acid, chlorophyll oil, cichoric acid, coenzyme Q10, copaiba oil, costmary oil, coumaric acid, cubeb oil, curcumin, davana oil, dexapanthenol, elemi oil, emu oil, ethyl cinnamate oil, eucalyptol oil, ferulic acid, frankincense oil, galangin, galbanum oil (greater galangal), geranium oil, shea butter, Ghana shea butter, ginger oil, glutamic acid, glutamine, glycerol, glycine, helichrysum oil, histidine, ho wood oil, hyaluronic acid, hydroxyproline, isoleucine, isopropyl myristate, jasmine oil, jojoba oil, kaempferia oil, kanuka oil, lauryl wax, lavandin oil, lavender oil, lecithin, lemon oil, lesser galangal, leucine, lidocaine, limonene oil, linalool, linalyl acetate, linoleic acid, litsea cubeba oil, lysine, magnesium sulfate, magnolia oil, marula oil, methyl sulfonyl methane (MSM), moringa oil, myristic acid, myrrh oil, neroli oil, nerolina oil, niacinamide, niola oil, Norwegian angelica oil, octanoic (caprylic) acid, oleanolic acid, oleic acid, oregano oil, orris oil, palmarosa oil, panthenol, paprika oleoresin, patchouli oil, peppermint oil, perilla seed oil, petitgrain oil, phellandrene, pine oil, praline, propylene glycol, protocatechuic acid, protocatechuic acid (PCA dihydroxybenzoic acid), purple carrot juice, ravensara oil, red beet juice, resinoid oil, rose hip seed oil, rosemary oil, sabinene, sea buckthorn oil, sericin, serine, sodium hyaluronate, sodium hydroxide, sodium lactate, sodium polyacrylate, sodium protocatechuic acid (sodium PCA), soy wax, spearmint oil, sulisobenzone, tamanu oil, terpinene oil, terpineol alcohol, threonine, thyme oil, thyme red oil, thyme white oil, thymohydroquinone methyl ether, thymoquinone, tri (polyglyceryl-3/lauryl) hydrogenated trilinoleate, turmeric oil (curcumin), ursolic acid, vitamin A, vitamin B3 (niacin), vitamin C, and winter savory is provided.

In another embodiment, a composition comprising one or more adjuvants selected from apricot kernal oil, argan nut oil, baobab oil, calendula oil, camellia oil, caprylic capric triglyceride, caryophyllene, elemi oil, kanuka oil, litsea cubeba oil, marula oil, palmarosa oil, palo santo, rose hip seed oil, sachi inchi oil, sea buckthorn oil, tamanu oil is provided.

In another embodiment, a composition comprising one or more adjuvants selected from acai berry, chamomile german oil, copaiba oil, linoleic acid, myrrh, oleic acid, rose hip seed oil is provided.

In another embodiment, a composition comprising one or more adjuvants selected from anethole anise camphor, cabreuva oil, carvacrol, floral, niaouli oil, rosemary oil, thyme red, white winter savory is provided.

In another embodiment, a composition comprising one or more adjuvants selected from benzyl alcohol, cetyl alcohol, panthenol is provided.

In another embodiment, a composition comprising one or more adjuvants selected from caffeic acid, kaempferol, protocatechuic acid is provided.

In another embodiment, a composition comprising one or more adjuvants selected from bees wax, benzoin, benzocaine, cedarwood essential oil, ferulic Acid, is provided.

In another embodiment, a composition comprising one or more adjuvants selected from carvone oil, geranium oil, ho wood oil, jasmine oil, lavandin oil, limonene oil, nerolina oil, peppermint, terpineol, and vetiver is provided.

In another embodiment, a composition comprising one or more adjuvants selected from alanine, arginine, glutamine, glycine, histidine, hydroxyproline, isoleucine, leucine, lysine, praline is provided.

In another embodiment, a composition comprising one or more adjuvants selected from vitamin A, vitamin B3, niacin, vitamin C, vitamin E, niacinamide is provided.

In another embodiment, a composition comprising one or more adjuvants selected from Agave nectar, distilled water, Sericin, Sodium Polyacrylate, 1-Tetradecanol is provided.

In another embodiment, a composition comprising one or more adjuvants selected from α-pinene, 1-tetradecanol, acai berry, agave, alanine, anethole anise camphor, apricot kernal oil, argan nut oil, arginine, soluble collagen, arctic fish (AF) collagen, baobab oil, bees wax, benzocaine, benzoin, benzyl alcohol, black cumin seed, cabreuva oil, caffeic acid, calendula oil, camellia oil, caprylic, capric triglyceride, carvacrol, carvone oil, caryophyllene, cedarwood essential oil, cetyl alcohol, chamomile german oil, copaiba oil, elemi oil, ferulic acid, geranium oil, Ghana shea butter, glutamine, glycerol, glycine, histidine, ho wood oil, hydroxyproline, isoleucine, jasmine oil, kaempferol, kanuka oil, lavandin oil, lecithin, leucine, limonene oil, linoleic acid, litsea cubeba oil, lysine, marula oil, methyl sulfonyl methane (MSM), myrrh, nerolina oil, niacinamide, niaouli oil, oleic acid, palmarosa oil, palo santo, panthenol, peppermint, praline, protocatechuic acid, rose hip oil, rosemary oil, sacha inchi oil, sea buckthorn oil, sericin, shea butter, sodium polyacrylate, tamanu oil, terpineol, thyme red, white, vetiver, vitamin A, vitamin B3 (Niacin), vitamin C, winter savory is provided.

In another embodiment, a composition comprising one or more adjuvants selected from aloe barbadensis (aloe vera) leaf extract, butyrospermum parkii butter (shea butter), alpha pinene, black cumin seed oil, tri (polyglyceryl-3/lauryl) hydrogenated trilinoleate, methyl sulfonyl methane (MSM), sodium hydroxide, betaine, carvacrol, magnesium sulfate, camellia oil, caryophyllene, caprylyl glycol, 1,2-hexanediol, apricot oil, argan oil, sea buckthorn oil, tamanu oil, sodium pyroglutamic acid (PCA), caprylic/capric triglyceride, copaiba oil, roman chamomile oil, linoleic acid, oleic acid, propylene glycol, ferulic acid, sodium lactate, baobab oil, marula oil, niacinamide PC, Protocatechuic (PCA), serine, alanine, glycine, tetrahexyldecyl ascorbate, tocopheryl acetate, retinyl palmitate, soluble collagen, arctic fish (AF) collagen, glutamic acid, lysine HCl, threonine, arginine, proline, tocopherol is provided.

In some embodiments, the composition of this disclosure comprises one or more of α-pinene, aloe vera inner leaf gel, shea butter, collagen, methyl sulfonyl methane, lecithin, glycerol, and one or more adjuvants of the present disclosure.

In some embodiments, the composition of this disclosure comprises from about 1.0 to about 5.0 wt. % α-pinene, from about 0.1 to about 1.0 wt. % 1-tetradecanol, from about 1.0 to about 5.0 wt. % collagen, from about 0.1 to about 3.0 wt. % agave nectar, from about 0.1 to about 2.0 wt. % alanine, from about 10.0 to about 20.0 wt. % aloe vera inner leaf gel, from about 0.1 to about 2.0 wt. % apricot kernal oil, from about 0.1 to about 2.0 wt. % argan nut oil, from about 0.1 to about 2.0 wt. % arginine, from about 0.1 to about 1.0 wt. % baobab oil, from about 1.0 to about 5.0 wt. % bees wax, from about 0.1 to about 2.0 wt. % benzoin, from about 1.0 to about 5.0 wt. % benzyl alcohol, from about 1.0 to about 5.0 wt. % black cumin seed oil, from about 0.1 to about 3.0 wt. % calendula oil, from about 0.1 to about 3.0 wt. % camellia oil, from about 0.1 to about 3.0 wt. % caprylic, capric triglyceride, from about 0.1 to about 3.0 wt. % carvacrol, from about 0.1 to about 3.0 wt. % caryophyllene, from about 1.0 to about 5.0 wt. % cetyl alcohol, from about 0.1 to about 3.0 wt. % chamomile german oil, from about 0.1 to about 3.0 wt. % copaiba oil, from about 20 to about 35.0 wt. % distilled water, from about 0.1 to about 3.0 wt. % elemi oil, from about 0.1 to about 3.0 wt. % ferulic acid, from about 0.1 to about 1.0 wt. % geranium oil, from about 10.0 to about 20.0 wt. % shea butter, from about 0.1 to about 3.0 wt. % glutamine, from about 0.1 to about 3.0 wt. % glycerol, from about 0.1 to about 2.0 wt. % glycine, from about 0.1 to about 2.0 wt. % histidine, from about 0.1 to about 1.0 wt. % ho wood oil, from about 0.1 to about 2.0 wt. % hydroxyproline, from about 0.1 to about 2.0 wt. % isoleucine, from about 0.1 to about 1.0 wt. % jasmine oil, from about 0.1 to about 1.0 wt. % kanuka oil, from about 0.1 to about 3.0 wt. % lavandin oil, from about 0.5 to about 4.0 wt. % lecithin, from about 0.1 to about 2.0 wt. % leucine, from about 1.0 to about 5.0 wt. % limonene oil, from about 0.1 to about 2.0 wt. % linoleic acid, from about 0.1 to about 2.0 wt. % litsea cubeba oil, from about 0.1 to about 2.0 wt. % lysine, from about 0.1 to about 1.0 wt. % marula oil, from about 0.5 to about 3.0 wt. % methyl sulfonyl methane, from about 0.1 to about 1.0 wt. % nerolina oil, from about 0.1 to about 1.0 wt. % niacinamide, from about 0.1 to about 2.0 wt. % oleic acid, from about 0.1 to about 1.0 wt. % palmarosa oil, from about 0.1 to about 3.0 wt. % panthenol, from about 0.1 to about 2.0 wt. % praline, from about 0.1 to about 3.0 wt. % protocatechuic acid, from about 0.1 to about 2.0 wt. % rosemary oil, from about 0.1 to about 2.0 wt. % sea buckthorn oil, from about 1.0 to about 5.0 wt. % sericin, from about 0.1 to about 3.0 wt. % sodium polyacrylate, from about 0.1 to about 2.0 wt. % tamanu oil, from about 0.1 to about 1.0 wt. % vitamin A, from about 0.1 to about 3.0 wt. % Vitamin B3 (niacin), from about 0.1 to about 1.0 wt. % vitamin C, and from about 0.1 to about 1.0 wt. % vitamin E.

α-Pinene is a hydrophobic compound found in nature. In one embodiment, α-pinene is provided as a colorless liquid with a pine, turpentine-like odor. In another embodiment, α-pinene is from a natural source. In yet another embodiment, α-pinene is from a synthetic source. α-pinene is reported to have a specific gravity at 25° C. of 0.8550-0.8600, a refractive index at 20° C. of 1.4640-1.4680, an optical rotation of 20.00-30.00. In one embodiment, α-pinene is provided without the presence of salmonella (AOAC, negative), lead, aflatoxin, arsenic, or mercury detected. In one embodiment, α-pinene is about 95% pure, preferably 99% pure, and more preferably about 99.5-100% pure. In one embodiment, pure α-pinene is obtained from Flavorchem Orchidia Fragrances, Downers Grove, IL, USA.

In one embodiment, a composition of the present disclosure comprises about 0.1 to about 50.0 wt. % α-pinene. In another embodiment, a composition of to the present disclosure comprises about 0.5 to about 20.0 wt. % α-pinene, about 1.0 to about 10.0 wt. % α-pinene, about 2.0 to about 7.0 wt. % α-pinene, or about 3.0 to about 4.0 wt. % α-pinene. In another embodiment, the composition of the present invention comprises 1.0 wt. % α-pinene, 2.0 wt. % α-pinene, 3.0 wt. % α-pinene, 4.0 wt. % α-pinene, 5.0 wt. % α-pinene, 6.0 wt. % α-pinene, 7.0 wt. % α-pinene, 8.0 wt. % α-pinene, 9.0 wt. % α-pinene, 10.0 wt. % α-pinene, 11.0 wt. % α-pinene, 12.0 wt. % α-pinene, 13.0 wt. % α-pinene, 14.0 wt. % α-pinene or 15.0 wt. % α-pinene.

In one embodiment, a composition of the present disclosure comprises from about 1.0 to about 5.0 wt. % α-pinene.

An “aloe vera” of the present disclosure may be prepared or otherwise derived from leaves of the aloe vera plant, otherwise known as Aloe barbadensis. In one embodiment, an aloe vera preparation is a hydrophilic component of a composition of the present disclosure. In one embodiment, an aloe vera preparation is and/or includes gel taken from the inner portion of aloe vera leaves (also known as “aloe vera inner leaf gel”), for instance as described above. Aloe vera inner leaf gel is a gel that comprises proteins, lipids, amino acids, vitamins, enzymes, inorganic compounds, organic compounds, and carbohydrates. The gel may be in its natural form, taken directly from the aloe vera leaf, or for instance in a processed form, for instance as a 10× or 200× concentrated dried powder. In another embodiment, an aloe vera preparation of this disclosure may include part of or all of whole aloe vera leaves, for instance in the form of aloe vera leaf juice or an aloe vera leaf extract. An aloe vera preparation that is or includes leaf juice or is or includes leaf extract may also be in a concentrated form, for instance in a 10× or 200× dried powder. In one embodiment, an aloe vera preparation of the present disclosure does not include the aloin or yellow or brown latex portion of the aloe leaf, and/or does not include the dark green outer covering of the leaf.

In one embodiment, the aloe vera inner leaf gel is in powder form, 200× concentrated. For instance, the 200× powder may be a spray-dried, light-cream-to-beige colored powder, having characteristics such as pH (1:199 w/w) 3.5-5.0, specific gravity (l: 199 w/w) 0.997-1.004, 8% maximum moisture, 100% particle size through 80 mesh, certifications such as is made from pure aloe i.e. does not include fillers and/or adulterants, and/or has no pathogens present (<10 cfu/g total plate count, <10 cfu/g yeast and mold). In one embodiment, the 200× aloe vera inner leaf gel powder is spray-dried to 200× the concentration of aloe vera inner leaf gel, is light cream to beige powder, pH 4.5, specific gravity (1:199 w/w) 1.001, about 4-5% moisture (water), 100% particle size through 80 mesh, is pure aloe i.e. does not include fillers and/or adulterants, and has no pathogens present (<10 cfu/g total plate count, <10 cfu/g yeast and mold). In a preferred embodiment, the aloe vera preparation is Terra-Pure™ Certified Spray Dried Aloe Vera Inner Leaf Powder Regular, 200× (Terry Laboratories, Melbourne FL, USA). In one embodiment, an aloe vera preparation including a 200× aloe vera preparation comprises or consists of inner leaf juice, made by removing the rind of the leaf prior to processing and then rinsing away aloe latex. The remaining, gelatinous inner-leaf material is then ground/crushed into aloe vera inner leaf juice.

In one embodiment, aloe vera according to the present disclosure includes other parts of the aloe vera plant. For instance, the aloe vera preparation may be a powder made from Aloe barbadensis leaf juice (e.g., CAS 94349-62-9). In one embodiment, leaf juice is extracted from fresh aloe leaves with a juice extractor, decolorized and filtered, separated via organic membrane. The membrane-separated concentrate juice is then freeze dried to be converted into the 200:1 Aloe Vera freeze dried gel powder. In one embodiment, an aloe vera preparation according to the present disclosure is Aloe Vera freeze dried gel powder 200:1 (Selco, Wald-Michelbach, Germany). In one embodiment, 200:1 aloe powder includes about 0.5% soluble solid of Aloe Vera gel. 200 kg of 1:1 Aloe Vera gel juice is frozen and dried into 1 kg of Aloe Vera freeze dried gel powder that becomes Aloe vera 200:1.

In one embodiment, aloe vera according to the present disclosure is a 200× powder produced through the water extraction of Aloe barbadensis leaf to prepare leaf juice powder. In one embodiment, aloe leaves are harvested, washed and filtered, aloin removed from filets, then the leaves ground, excess pulp removed, treated with heat, filtered, and spray dried. Aloe vera according to the present disclosure, in unconcentrated or concentrated (e.g., 200×) form, is a hydrophilic component/ingredient of this disclosure. While 200× concentrated powders are a preferred embodiment according to the present disclosure, other concentrations may also be included in the present disclosure.

In one embodiment, aloe vera preparations of the present disclosure, for instance provided by Terry Laboratories as described above, include aloe polysaccharides consisting of linear (unbranched) chains of b-1-4-linked glucose and mannose molecules (often called glucomannans, and because more mannose than glucose is present, polymannans). These linear chains range from a few molecules to several thousand molecules. By convention, the lower limit is usually taken as a molecular weight of about 1,000 Daltons for the material to qualify as a polysaccharide. In one embodiment, aloe vera preparations for instance as provided by Terry Laboratories are microbiologically clean, color stable, processed as quickly as possible following harvest, and processed at the lower temperature possible. Also, in one embodiment the aloe vera preparation including for instance 10× or 200× aloe vera preparations retains polysaccharide molecular weight distribution comparable to unprocessed aloe vera and reduces polysaccharide degradation such that not less than 50% of polysaccharides are greater than 1 million Daltons. In one embodiment, an aloe vera preparation includes acemannan (beta-1-4-acetylated glucomannans).

In one embodiment, a composition of the present disclosure comprises about 0.1 to about 50.0 wt. % aloe vera. In another embodiment, a composition according to the present invention includes about 5.0 to about 30 wt. % aloe vera, about 10.0 to about 20.0 wt. % aloe vera, about 13.0 to about 18.0 wt. % aloe vera, about 14.0 to about 16.0 wt. % aloe vera, or about 15.0 wt. % aloe vera. In one embodiment, the aloe vera preparation may have its natural concentration, 1×, single-strength equivalent form, or in a concentrated 10×, 200×, or other concentrated form.

In one embodiment, the composition of the present disclosure comprises “shea butter.” Shea butter and/or oil extracted from shea butter (“shea butter oil”), is prepared from or otherwise derived from any Shea tree, including but not limited to a Shea tree from Ghana. In one embodiment, shea butter or shea butter oil is Butyrospermum parkii butter (CAS 194043-92-0; EC 293-515-7); Butyrospermum parkii oil (CAS 91080-23-8, EC 293-515-7), and/or vegetable oil (CAS 68956-68-3, EC 273-313-5). Other shea butters and shea butter oils may be used in shea butter preparations of this invention, including those having different CAS or EC, or other identifying numbers.

In one embodiment, Shea butter oil is prepared by a series of processes including mechanical extraction, solvent extraction, refinement, deodorization, and solvent fractionation. In another embodiment, Shea tree nuts undergo mechanical pressing and solvent extraction with hexane to prepare crude oil, which is then subjected to degumming with acidified water (phosphoric or citric acid) (degumming removes phospholipids, metals, and proteins), neutralization with alkaline water (sodium hydroxide) (neutralization removes free fatty acids, metals, and proteins), and bleaching with bleaching earth (clay) and citric acid (bleaching removes pigments, metals, and proteins) to prepare a bleached oil. Then, the bleached oil is mixed with solvent and cooled (separating solid and liquid constituents), filtered, solvent (hexane and/or acetone) recovered; and then deodorized with steam (removes flavors, free fatty acids, oxidation products and residual solvent). In one embodiment, Shea butter oil according to the present preparation is liquid at room temperature, has a red color, a density (25° C.) of 0.9-0.94 g/cm³, a fatty acid composition (w/w) for instance as follows: 5% C16:0, 28% C18:0, 56% C18:1, 9% C18:2, 0.3% C18:3, 1% C20:0, 1% C22:0, available as Lipex SheaLiquid™ (AAK Sweden AB, Karlshamn, Sweden, SE-374-82).

In one embodiment, a composition of the present disclosure comprises about 10.0 to about 20.0 wt. % aloe vera inner leaf gel.

In one embodiment, the composition of the present disclosure comprises about 0.1 to about 50.0 wt. % shea butter. In a preferred embodiment, the composition of the present disclosure comprises about 1.0 to about 30.0 wt. % shea butter, about 8.0 to about 20.0 wt. % shea butter about 12.0 to about 18.0 wt. % shea butter, about 14.0 to about 16.0 wt. % shea butter, or about 15.0 wt. % shea butter. In one embodiment, the composition of the present disclosure comprises about 1.0 to about 20.0 wt. % shea butter, wherein the preparation is shea butter oil, about 2.0 to about 15.0 wt. % w/w shea butter oil, about 3.0 to about 8.0 wt. % shea butter oil, or about 4.0 to about 6.0 wt. % shea butter oil.

In one embodiment, a composition of the present disclosure comprises from about 10.0 to about 20.0 wt. % shea butter.

In one embodiment, the composition of the present disclosure comprises soluble collagen. In one embodiment, soluble collagen, such as Ichtyocollagen™, comprises native fish collagen. In one embodiment, Ichtyocollagen™ has film-forming and moisturizing, skin conditioning properties. In one embodiment, Ichtyocollagen™ is provided in liquid form at 25° C., colorless to pale yellow, with appearance clarity of clear to slightly opalescent. In one embodiment, ichtyocollagen comprises 0.4-0.7% protein (by BCA), pH 3.5-4.5, specific gravity at 20° C. of 1.000-1.020, refractive index at 25° C. of 1.34-1.36, hydroxyproline content 0.06-0.09%, and/or maximum 100 CFU/G total aerobic microbial count and maximum 10 CFU/G total combined yeast/mold count. In one embodiment, Ichtyocollagen™ comprises soluble collagen (INCI), CAS 9007-34-5, EINECS 232-697-4, as well as propylene glycol (approx. 10%), phenoxyethanol (1.5%), potassium sorbate (0.05%), trisodium EDTA (0.1%). In one embodiment, Ichtyocollagen™ includes about 88% of natural origin content. Another embodiment, the composition of the present disclosure comprises Ichtyocollagen™ PH (Croda/Sederma Inc., Edison, NJ, USA)

In one embodiment, a composition of the present disclosure comprises from about 1.0 to about 5.0 wt. % collagen, from about 3.0 to about 10.0 wt. % collagen, from about 8.0 to about 115.0 wt. % collagen, and, from about 10.0 to about 25.0 wt. % collagen.

In one embodiment, a composition of the present disclosure comprises from about 0.5 to about 3.0 wt. % methyl sulfonyl methane.

In one embodiment, a composition of the present disclosure comprises from about 1.0 to about 5.0 wt. % black cumin seed oil.

In one embodiment, a composition of the present disclosure comprises from about 0.5 to about 4.0 wt. % lecithin.

In one embodiment, a composition of the present disclosure comprises from about 0.1 to about 3.0 wt. % glycerol.

The present disclosure also provides pharmaceutical formulations of the disclosed compositions. The pharmaceutical formulations comprise a therapeutically effective amount of a composition of the present disclosure formulated with one or more pharmaceutically acceptable carriers and/or excipients.

In one embodiment, the term “pharmaceutically acceptable” means approved by a regulatory agency of the Federal or a state government or listed in the U.S. Pharmacopeia or other generally recognized pharmacopeia for use in animals and, more particularly, in humans.

In another embodiment, the term “pharmaceutically acceptable” means suitable for use in mammals.

The pharmaceutical compositions of the present invention may be formulated for administration by a variety of routes including oral, rectal, transdermal, parenteral (subcutaneous, intraperitoneal, intravenous, intraarterial, transdermal and intramuscular), topical, intranasal, or via a suppository. Such compositions are prepared in a manner well known in the pharmaceutical art and comprise as an active ingredient at least one compound of the present invention as disclosed herein, and a pharmaceutically acceptable excipient or carrier.

The compounds of the present disclosure and pharmaceutically acceptable salts thereof may be in combination with one or more pharmaceutical excipients to form various types of formulations for delivery. For example, topical formulations may be used and may include pharmaceutically acceptable preservatives, surfactants, viscosity enhancers, buffers, sodium chloride, and water to form aqueous pharmaceutically compatible solutions and suspensions. Suitable topical formulations are known in the art, for example, as described in text books and review articles, e.g., Benson, et al., Topical and Transdermal Drug Delivery: From Simple Potions to Smart Technologies, Current Drug Delivery, 2019, 16(5) pgs. 444-460. Suitable ophthalmological formulations are also known in the art and described in text books and review articles, e.g., “Recent Advances in Topical Ocular Drug Delivery”, by V. K. Yellepeddi and S. Palakurthi (J. Ocul. Pharmacol. Ther. 2016, 32(2): 67-82), herein incorporated by reference in its entirety. Systemic formulations (for example, orally ingested tablets) and formulations for intraocular injection are also contemplated, (pharmaceutically acceptable carriers). Examples of suitable pharmaceutically acceptable excipients and carriers are described in Remington's Pharmaceutical Sciences, Mack Publishing Company, a standard reference text in the pharmaceutical field, herein incorporated by reference in its entirety.

Pharmaceutical excipients may be any excipient known in the art and are limited only by chemical-physical considerations, such as solubility and lack of reactivity with the compound for use in the present disclosure, and by the route of administration. The choice of excipient is determined by the particular method/route used to administer the pharmaceutical composition. Some examples of suitable pharmaceutical excipients include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose.

The formulations of the present disclosure may additionally include lubricating agents such as talc, magnesium stearate, and mineral oil, wetting agents, surfactants, emulsifying and suspending agents, preserving agents such as methyl- and propylhydroxybenzoates, sweetening agents, flavoring agents, colorants, buffering agents (e.g., acetates, citrates or phosphates), disintegrating agents, moistening agents, antibacterial agents, antioxidants (e.g., ascorbic acid or sodium bisulfite), chelating agents (e.g., ethylenediaminetetraacetic acid), and agents for the adjustment of tonicity such as sodium chloride.

Other pharmaceutical excipients of the present disclosure may be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. In one embodiment, water is a preferred excipient when the pharmaceutical composition is administered intravenously. Saline solutions and aqueous dextrose and glycerol solutions may also be employed as liquid excipients, particularly for injectable solutions.

In one embodiment, the term “pharmaceutically acceptable salts” refers to either “pharmaceutically acceptable acid addition salts” or “pharmaceutically acceptable basic addition salts”. The term “salt” or “salts” may refer to “pharmaceutically acceptable salts” or to salts suitable for use in industrial processes, that might not be pharmaceutically acceptable. The term “pharmaceutically acceptable acid addition salts” refer to non-toxic organic or inorganic acid addition salt of the base compounds disclosed herein or any of their intermediates. Illustrative inorganic acids which form suitable salts include hydrochloric, hydrobromic, sulphuric, and phosphoric acid and acid metal salts such as sodium monohydrogen orthophosphate, and potassium hydrogen sulfate. Illustrative organic acids, which form suitable salts include the mono-, di-, and tricarboxylic acids. Illustrative of such acids are for example, acetic, glycolic, lactic, pyruvic, malonic, succinic, glutaric, fumaric, malic, tartaric, citric, ascorbic, maleic, hydroxymaleic, benzoic, hydroxy-benzoic, phenylacetic, cinnamic, salicylic, 2-phenoxybenzoic, p-toluenesulfonic acid, and sulfonic acids such as methane sulfonic acid and 2-hydroxyethane sulfonic acid. Such salts may exist in either a hydrated or substantially anhydrous form. In general, the acid addition salts of these compounds are soluble in water and various hydrophilic organic solvents. The term “pharmaceutically acceptable basic addition salts” refers to non-toxic organic or inorganic basic addition salts of the compounds disclosed herein. Illustrative bases which form suitable salts include alkali metal or alkaline-earth metal hydroxides such as sodium, potassium, calcium, magnesium, or barium hydroxides; ammonia, and aliphatic, alicyclic, or aromatic organic amines such as methylamine, dimethylamine, trimethylamine, and picoline.

The compositions of the present disclosure also comprise solvates of the compounds herein. “Solvates” include physical associations of a compound of the disclosure with one or more solvent molecules. This physical association involves varying degrees of ionic and covalent bonding, including hydrogen bonding. In certain instances, the solvate may be capable of isolation. The term “solvate” encompasses both solution-phase and isolatable solvates. Non-limiting examples of suitable solvates include ethanolates, methanolates and the like. A “hydrate” is a solvate wherein the solvent molecule is water.

The meaning of any range disclosed as a whole number (e.g., 1 or 12, or 33) is understood to include the whole numbers between the stated range. For example, the range of about 10 to about 20 discloses the ranges about 10 to about 11, about 11 to about 12, about 12 to about 13, about 13 to about 14, about 14 to about 15, about 15 to about 16, about 16 to about 17, about 17 to about 18, about 18 to about 19, and about 19 to about 20.

The meaning of any range disclosed to one-tenth (e.g., 0.0) is understood to include the whole numbers and one-tenth numbers between the stated range. For example, the range of about 0.0 to about 1.0 discloses the ranges about 0.0 to about 0.1, about 0.1 to about 0.2, about 0.2 to about 0.3, about 0.3 to about 0.4, about 0.4 to about 0.5, about 0.5 to about 0.6, about 0.6 to about 0.7, about 0.7 to about 0.8, about 0.8 to about 0.9, and about 0.9 to about 1.0.

The compositions of the present disclosure may include one or more buffering agent(s) or pH adjusting agent(s) to provide improved pH control. In certain embodiments, a composition of this disclosure has a pH between about 5.0 and about 8.0, between about 5.0 and about 7.0, between about 6.0 and about 8.0, or between about 6.0 and about 7.0. In one embodiment, the pH of a composition of this disclosure is from about 5.3 to about 7.3. In one embodiment, the pH of a composition of this disclosure is from about 5.3 to about 6.3. In one embodiment, the pH of a composition of this disclosure is from about 6.3 to about 7.3. In one embodiment, a composition of this disclosure has an approximately neutral pH of about 6.8 to about 7.2. In another embodiment, the pH of a composition of this disclosure is from about 5.3 to about 5.8. In yet another embodiment, the pH of a composition of the present disclosure is about 5.5. In yet another embodiment, the pH of a composition of the present disclosure is optimized as known by those of skill in the art, see, e.g., Lukic, et al., Towards Optimal pH of the Skin and Topical Formulations: From the Current State of the Art to Tailored Products, Cosmetics, 2021, 8, 69, pgs. R1-19.

In one embodiment, at least one of the excipients is a non-aqueous excipient.

Compositions of the present invention may further comprise optional components such as lipid carriers such as oils or waxes so as to provide a semi-solid consistency as desired. Additional inactive excipients such as preservatives, antioxidants, perfumes, and the like may also be added.

Oils that may be used as optional components in the compositions of the present invention include, but are not limited to, petrolatum (petroleum jelly), vegetable oil, fruit oil, plant oil, animal oil, mineral oil, coconut oil, olive oil, lanolin, peanut oil, hydrogenated and sulphated oils such as cottonseed oil, soybean oils, almond oil, sesame oil, and the like. In another embodiment, the oil comprises corn oil, peanut oil, coconut oil, grape seed oil, sunflower oil, lemon oil, orange oil, peppermint oil, palm kernel oil, castor oil, hydrogenated cottonseed oil, hydrogenated soy oil, hydrogenated soybean oil, hydrogenated vegetable oil, partially hydrogenated soybean oil, partially hydrogenated palm oil, hydrogenated castor oil, light mineral oil, mineral oil, or a combination thereof.

Waxes that may be used as optional components in the compositions of the present invention include, but are not limited to, paraffin wax, beeswax, carnauba wax, cetyl ester wax, microcrystalline wax, spermaceti wax, and the like.

Other contemplated excipients, which may be utilized in the pharmaceutical compositions of this disclosure include, for example, antimicrobial agents, antioxidants, antistatic agents, lipids such as phospholipids or fatty acids, steroids such as cholesterol, protein excipients such as serum albumin (human serum albumin), recombinant human albumin, gelatin, casein, salt-forming counterfoils such sodium and the like. These and additional known pharmaceutical excipients and/or additives suitable for use in the formulations of this disclosure are known in the art, e.g., as listed in “The Handbook of Pharmaceutical Excipients, 4th edition, Rowe et al., Eds., American Pharmaceuticals Association (2003); and Remington: the Science and Practice of Pharmacy, 21st edition, Gennaro, Ed., Lippincott Williams & Wilkins (2005).

In yet another embodiment, the composition is prepared for topical administration. In one embodiment, the topical composition comprises an ointment. In another embodiment, the topical composition comprises a cream. In another embodiment, the topical composition comprises a lotion. In another embodiment, the topical composition comprises a balm. In another embodiment, the topical composition comprises a gel. In another embodiment, the topical composition comprises a paste. In another embodiment, the topical composition comprises a drop. In another embodiment, the topical composition comprises a foam. In another embodiment, the topical composition comprises a powder. solution. In another embodiment, the topical composition comprises a spray. In another embodiment, the topical composition comprises a patch.

The components of the topical formulation are admixed under various conditions, including sterile conditions, with a pharmaceutically acceptable excipient and any needed preservatives or buffers as may be required.

For topical administration to body surfaces using, for example, ointment, cream, lotion, balm, gel, paste, drop, foam, powder, solution, spray, or patch and the like, the compositions of the present disclosure can be prepared and applied in a physiologically acceptable diluent with or without a pharmaceutical carrier. The ointment, cream, lotion, balm, gel, paste, drop, foam, powder, solution, spray, or patch of the present disclosure may contain, in addition to the active compounds of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Adjuvants for topical formulations may include, for example, sodium carboxymethylcellulose, polyacrylates, polyoxyethylene-polyoxypropylene-block polymers, polyethylene glycol and wood wax alcohols.

Powders and sprays may contain, in addition to the compounds of this invention, excipients such as lactose, talc, silicic acid, aluminum hydroxide, calcium silicates and polyamide powder, or mixtures of these substances. Sprays may additionally contain customary propellants such as chlorofluorohydrocarbons.

[Emulsions] In one embodiment, a composition of the present disclosure is a water-in-oil emulsion comprising water and an emulsifier. In one embodiment, water is distilled water, and comprises about 25 to about 70 wt. % of the emulsion and the composition. In an embodiment, water comprises about 25 to about 50 wt. % or about 50 to about 70 wt. % of the emulsion. In another embodiment, an emulsifier is tri-(polyglyceryl-3/lauryl) hydrogenated trilinoleate (Cithrol PGTL), polyglyceryl-3 sorbityl linseedate (Sinerga), polyglyceryl-2 oleate, polyhydroxystearic acid, polyglyceryl-2 stearate (Innovacos), Polyglyceryl-3 Triolivate (Acme Hardesty), polyglyceryl-3 polyricilinoleate (IOI Oleo), polyglyceryl-2 dipolyhydroxystearate (BASF), polyglyceryl-2 sesquiisostearate (Clariant), polyglyceryl-3 diisostearate (Gattefosse, BASF), polyglyceryl-6 polyricinoleate (and) polyglyceryl-3 diisostearate (and) Disteardimonium Hectorite (Elementis), Olive Oil Polyglyceryl-6 Esters (and) Polyglyceryl-6 Pentaoleate (Coast Southwest), Polyglyceryl-6 Polyricilinoleate (and) Polyglyceryl-10 Dioleate (Grant), Cetyl PEG/PPG-10/1 Dimethicone (Evonik), Methylglucose Sesquioleate (Lubrizol), and/or Octyldodecanol (and) octyl dodecyl xyloside (and) PEG-30 dipolyhydroxystearate (Easynov).

In one embodiment, a water-in-oil emulsion of the present disclosure comprises about 0.1 to about 10.0 wt. % emulsifier, about 0.5 to about 5.0 wt. % emulsifier, about 1.0 to about 3.0 wt. % emulsifier, or about 1.0 to about 2.0 wt. % emulsifier. In another embodiment, the water-in-oil emulsion is a topical composition.

Alternative formulations include nasal sprays, liposomal formulations, dendrimer formulations, slow-release formulations, controlled-release formulations, and the like, as are known in the art. The use of liposomal formulations is known in the art and have been described for use in the development of topical formulations, see. e.g., Castaneda-Reyes, et al., Development, Characterization and Use of Liposomes as Amphipathic Transporters of Bioactive Compounds for Melanoma Treatment and Reduction of Skin Inflammation: A Review, Int J Nanomedicine, 2020, 15, pgs. 7627-7650. The use of dendrimer formulations is known in the art and have been described for use in the development of topical formulations, see. e.g., Pentek, et al., Development of a Topical Resveratrol Formulation for Commercial Applications Using Dendrimer Nanotechnology, Molecules, 2017, 22(1), pg. 137.

Another formulation employed in the methods of the present disclosure employs transdermal delivery devices (“patches”). Such transdermal patches may be used to provide continuous or discontinuous infusion of the compounds of the present disclosure in controlled amounts. The construction and use of transdermal patches for the delivery of pharmaceutical agents is well known in the art. Transdermal patches have the added advantage of providing controlled delivery of a compound to the body. Such dosage forms may be made by dissolving or dispensing the components disclosed herein in a proper medium. Absorption enhancers may also be used to increase the flux of the compound across the skin. The rate of release may also be controlled by either providing a rate controlling membrane or by dispersing the compound in a polymer matrix or gel.

One embodiment of the present disclosure is a method for the alleviation of symptoms and/or the treatment of palmar fascia fibromatosis. Another embodiment of the present disclosure is a method for the alleviation of symptoms and/or the treatment of Dupuytren's disease. Yet another embodiment of the present disclosure is a method for the alleviation of symptoms and/or the treatment of Dupuytren's contracture.

One embodiment of the present disclosure is a method for the alleviation of symptoms and/or the treatment of Ledderhose's disease.

One embodiment of the present disclosure is a method for the alleviation of symptoms and/or the treatment of Peyronie's disease.

One embodiment of the present disclosure is a method for the alleviation of symptoms and/or the treatment of scleroderma.

One embodiment of the present disclosure is a method for the alleviation of symptoms and/or the treatment of Raynaud's (or Renaud's) phenomenon.

One embodiment of the present disclosure is a method for the alleviation of symptoms and/or the treatment of wound healing, such as skin damage from cuts, abrasions and burns, as well as aging skin changes.

One embodiment of the present disclosure is a method for the alleviation of symptoms and/or the treatment of toe nail fungus.

One embodiment of the present disclosure is a method for the alleviation of symptoms and/or the treatment of chemotherapy and/or radiation induced oral mucosal ulceration.

One embodiment of the present disclosure is a method for the alleviation of symptoms and/or the treatment of aphthous ulcers.

One embodiment of the present disclosure is a method for the alleviation of symptoms and/or the treatment of fibrotic processes involved in wound healing including topical injuries and wounds from surgical procedures.

One embodiment of the present disclosure is a method for the alleviation of symptoms and/or the treatment of topical injuries including abrasions, acute sun burns, cat scratches, chemical burns, dog bites, finger/toe nail damage, heated object burns, spider and insect bites and superficial cuts.

One embodiment of the present disclosure is a method for the alleviation of symptoms and/or the treatment of topical disorders including cutaneous rashes due to allergic reactions, poison ivy, poison oak, secondary infectious rashes, including measles and chicken pox.

One embodiment of the present disclosure is a method for the alleviation of symptoms and/or the treatment of wounds due to surgical procedures including all wounds made in and through the skin, emergent first aid, regardless of type of surgery, skin grafts, and sutured and non-sutured incisions.

One embodiment of the present disclosure is a method for the alleviation of symptoms and/or the treatment of disorders including aged skin, sun exposed skin surfaces, and baldness.

Fibrotic processes involved in wound healing may include topical injuries or surgical procedures. Typical topical injuries include abrasions, acute sun burns, cat scratches, chemical burns, dog bites, finger/toe nail damage, heated object burns, spider and insect bites and superficial cuts. Other topical disorders include cutaneous rashes due to allergic reactions, poison ivy, poison oak, secondary infectious rashes, including measles and chicken pox. Wounds due to surgical procedures include all wounds made in and through the skin, emergent first aid, regardless of type of surgery, skin grafts, and sutured and non-sutured incisions. Other disorders contemplated by the present invention include aged skin, sun exposed skin surfaces, and baldness.

In one embodiment, the present invention may be used topically or transdermally.

In one embodiment, the term “administering” or “administration” refers to bringing into contact with a composition of the present disclosure. In one embodiment, the compositions are applied locally. In one embodiment, the compositions are applied topically. In one embodiment, the compositions are applied mucosally, e.g., to a mucous membrane. Administration can be accomplished to organs, cells, tissue cultures, or to living organisms, for example a human.

In one embodiment, a “therapeutic” treatment is the administration of a composition of the present disclosure to a subject experiencing or exhibiting signs or symptoms of the diseases or disorders disclosed herein for the purpose of diminishing or eliminating those diseases, disorders and/or their symptoms.

According to any of the methods of the present invention and in one embodiment, the subject is mammalian. In another embodiment, the subject is human. In another embodiment, the subject is a primate, which in one embodiment, is a non-human primate. In another embodiment, the subject is murine, which in one embodiment is a mouse, and, in another embodiment is a rat. In another embodiment, the subject is canine, feline, bovine, equine, caprine, ovine, porcine, simian, ursine, vulpine, or lupine. In one embodiment, the subject is a chicken or a fish.

In some embodiments, the composition of the present invention comprises a therapeutically effective amount of one or more of the compounds disclosed herein, or a pharmaceutically acceptable salt or solvate thereof, and at least one pharmaceutically acceptable excipient. In one embodiment, a “therapeutically effective amount” of a composition of the present disclosure is an amount of the composition that is sufficient to provide a beneficial effect to the subject to which the composition is administered.

In one embodiment of the disclosure, the pharmaceutical composition is administered locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material. According to some embodiments, administration can be by direct injection, e.g., via a syringe, at the site of a tumor or neoplastic or pre-neoplastic tissue.

In one embodiment, the compositions of the present disclosure may be administered in combination with a biodegradable, biocompatible polymeric implant, which releases the compound over a controlled period of time at a selected site. Examples of polymeric materials include polyanhydrides, polyorthoesters, polyglycolic acid, polylactic acid, polyethylene vinyl acetate, copolymers and blends thereof (See, e.g., Medical Applications of Controlled Release, Langer and Wise (eds.), 1974, CRC Pres., Boca Raton, Fla.). In yet another embodiment, a controlled release system may be placed in proximity of the site to be administered.

In other embodiments, the compositions of the present disclosure may be administered by any convenient route, for example by infusion or bolus injection, by absorption through epithelial linings (e.g., oral mucosa, rectal and intestinal mucosa, etc.), and may be administered together with other therapeutically active agents.

The therapeutic effect of the compositions and formulations disclosed herein may be objective (i.e., measurable by some test or marker) or subjective (i.e., subject provides or evidences an indication of treatment for example the alleviation of symptoms related to the disorder). Effective doses will vary depending on the route of administration, in addition to the usage with other pharmaceutical and/or medicinal agents. The specific therapeutically effective dose level for any particular patient will depend upon a variety of factors including the disorder being treated, the severity of the disorder, the activity of the composition employed, the specific composition employed, the age, body weight, general health, sex and diet of the subject, the time and frequency of administration, the route of administration, the rate of excretion of the specific composition employed, the duration of the treatment, and additional medications or treatments used in combination or contemporaneously with the specific composition employed; and like factors well known in the art.

In one embodiment, “amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

The amount and duration of treatment of a composition of the present disclosure that will be effective for a particular fibrotic disease or disorder will depend on the nature of the disease or disorder, and may be determined using clinical techniques known to those of skill in the art. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may also be extrapolated from dose-response curves derived from in vitro or animal model bioassays or systems.

In one embodiment, the composition of the present disclosure is administered once per day. In another embodiment, the composition of the present disclosure is administered twice per day. In another embodiment, the composition of the present disclosure is administered three times per day. In another embodiment, the composition of the present disclosure is administered four times per day. In another embodiment, the composition of the present disclosure is administered hourly. In another embodiment, the composition of the present disclosure is administered once every two days, once every three days, twice a week, once a week, once every 2 weeks, once every 3 weeks.

In one embodiment, the composition of the present disclosure is administered for 7 days to 28 days. In another embodiment, the composition of the present disclosure is administered for 7 days to 8 weeks. In another embodiment, the composition of the present disclosure is administered for 7 days to 50 days. In another embodiment, the composition of the present disclosure is administered for 7 days to six months. In another embodiment, the composition of the present disclosure is administered for 7 days to one and half years. In another embodiment, the composition of the present disclosure is administered for 14 days to 12 months. In another embodiment, the composition of the present disclosure is administered for 14 days to 3 years. In another embodiment, the composition of the present disclosure is administered for several years. In another embodiment, the composition of the present disclosure is administered for one month to six months. In another embodiment, the composition of the present disclosure is administered until the subject no longer experiences or exhibits symptoms of the fibrotic disease or disorder.

Another embodiment is a kit for the treatment of the fibrotic diseases and disorders of the present disclosure.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

### EXAMPLES

### Experimental Procedures

The following examples are presented in order to more fully describe the compositions and processes of the present disclosure, which are intended only as illustration and not limiting of the scope of this disclosure. Various alterations and modifications to the disclosed examples and embodiments will be apparent to those skilled in the art and such alterations and modifications including, without limitation, those relating to the processes, formulations and/or methods of this disclosure may be made without departing from the scope of the disclosure and of the claims.

### Example 1

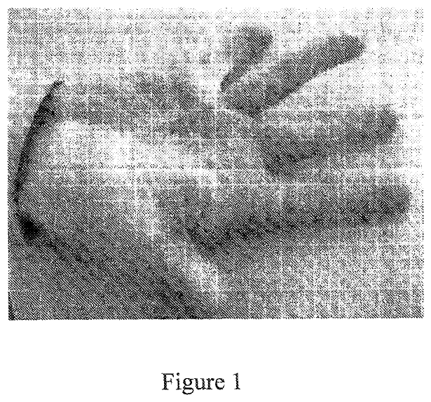

Dupuytren's Disease of the hands is an early form of what most likely will evolve overtime into Dupuytren's Contracture of the hands. This relatively infrequent disease is based on northern European ancestry that increases in incidence typically from age 45-50 years of age with increasing incidence and severity of existing disease throughout the rest of these people's lives. There is a mild increase in the incidence of males over females in the progression of the disease. The earlier in life it begins the expectation of more accelerated progression, and the involvement of more fingers will be experienced. It converts from Dupuytren's Disease to Dupuytren's Contracture when the fingers begin to bend in towards the palms. The abnormality begins as the Disease with the discovery of a subcutaneous nodule on the palmar surface of the hand that is usually not tender at the base of fingers 3, 4, and 5 on either hand. After a relatively short time, abnormal skin folds begin to develop below the nodule into the palm. Additional nodules may form then or at any time in the future on either hand mostly involving fingers 3, 4, and 5. Another example of involvement of any finger is observed by placing the palm on a flat surface and determining the ability to raise the affected finger up off the table. Over time, the involved fingers will not be able to rise above the flat surface. Progression of this problem is expected for any involved finger in Dupuytren's Disease to Dupuytren's contracture whenever the angle between any finger and the palm begins to decrease from vertical with the hands held upright. Ongoing disease progression of Dupuytren's Contracture will be noted with further movement of the fingers into the palm, as shown in FIG. 1. This degree of curvature is measured to document ongoing progression of the disease.

The disclosed composition halts the progression of Dupuytren's disease to the

Dupuytren's contracture stage. All nodules show significant decrease in size which appears to happen within the first 2-4 months of treatment.

### Example 2

White male, 69 years old, developed palm nodules and skin folds at base of both left and right hands with rigid third digits in 2014. When the left hand had progressed to bend towards the palm at 5 degrees in 2015, the first preliminary product formulation used twice daily, thoroughly rubbing it into the skin, made nodules disappear and stopped progression of contracture of third digit. The composition was applied to both fingers twice a day. Within a few weeks of using the composition, the nodule size and thickness began to decrease, first on the left hand and then followed on the right hand. More importantly, the left fourth finger had returned back to vertical, reversing from the early Contracture Stage back to the Disease Stage of Dupuytren's. Many months usage of the product kept these progressions limited. However, when no product was available for 6 months in 2017, ventral surface cords developed with new chords extending into the palm on both the left and right hands extending from the fourth fingers down into the palm. When product became available again, the previous progression of these lesions halted with continuous usage of this product twice a day up to November 2018. Current observations show decreased skin folds, softening of the flexor chords, and no new contractures noted with continued twice a day usage.

### Example 3

White male, 66 years of age developed skin nodules and skin folds on left and right third digits at the base. Both nodules disappeared and skin folds decreased with 6 months of twice a day applications.

### Example 4

A small clinical trial for the use of the disclosed composition for patients with active Dupuytren's Disease and Contracture was conducted in Ukraine with two patients.

The first is a 73 year old male, 5 foot 6 inches weighing 139 pounds who had untreated contractures on the right hand for 11 years and on the left hand for 16 years. Prior to treatment, the right hand had a fifth finger Stage 3 contracture with 100° of flexion and a left hand fifth finger Stage 4 contracture with 150° of flexion. After 6 weeks of treatment with the disclosed composition, the right hand fifth finger contraction reduced to a Stage 2 contracture with 55° of flexion, but the left hand Stage 4 contraction treated for the same time remained a stage 4 lesion that only reduced to a 140° of flexion.

The second patient in this preliminary trial was also a 73 year old male, 5 foot 8 inches weighing 194 pounds who had untreated contractures on the right fifth digit Stage I with 25° of flexion and a fourth digit Stage 2 with 75° of flexion for 13 years. The left hand had a fifth digit Stage 3 contraction with 95° of flexion and a fourth finger Stage 4 contraction with 140° contraction for 9 years. After 6 weeks of treatment with the disclosed composition, the right hand fifth digit Stage 1 lesion with 25° of flexion remained a Stage 1 but no longer had any degrees of flexion. The right hand fourth digit Stage 2 contracture with 75° flexion reduced to 55° of flexion. The left hand fifth digit Stage 3 contracture with 95° of flexion reduced to a Stage 2 contraction with 45° of flexion. The left hand fourth finger Stage 4 contraction with 140° of flexion reduced to a Stage 3 contracture with 110° of contraction.

Table 5 lists the composition formulation for treatment of Dupuytren's Disease and Contracture.

TABLE 5
Dupuytren's Disease and Contracture Composition Formulation

| Hydrophobic | | | Hydrophilic | | |
|---|---|---|---|---|---|
| Components | amount | | Components | amount | |
| a-Pinene (APN) | 60 | ml | 1-Tetradecanol | 2.5 | g |
| Apricot Kemal Oil | 5 | ml | AF (Artie Fish) | 40 | g |
| (AKO) | | | Collagen (AFC) | | |
| Argan Nut Oil (AGN) | 5 | ml | Agave nectar (AGV) | 10 | ml |
| Baobab Oil (BAB) | 2.5 | ml | Alanine (ALA) | 5 | g |
| Bees Wax (BW) | 40 | g | Aloe Vera Leaf Gel | 275 | g |
| | | | (AVLG) | | |
| Benzoin (BZN) | 4 | ml | Arginine (ARG) | 5 | g |
| Benzyl Alcohol (BA) | 50 | ml | Distilled water | 500 | ml |
| Calendula Oil (CDA) | 5 | ml | Ferulic Acid (FRA) | 10 | g |
| Camellia Oil (CMA) | 10 | ml | Glutamine (GLU) | 5 | g |
| Capaiba Oil (CPO) | 10 | ml | Glycine (GLY) | 5 | g |
| Caprylic, Capric | 10 | ml | Histidine (HST) | 5 | g |
| Triglyceride (CCT) | | | | | |
| Carvacrol (CVC) | 5 | ml | Hydroxyproline (HDP) | 5 | g |
| Caryophyllene (CAP) | 10 | ml | Isoleucine (ISL) | 5 | g |
| Cetyl Alcohol (CTA) | 40 | g | Lecithin (LCT) | 25 | g |
| Chamomile German | 10 | ml | Leucine (LUC) | 5 | g |
| Oil (CGB) | | | | | |
| Elemi Oil (EMO) | 5 | ml | Linoleic Acid (LOA) | 5 | ml |
| Geranium Oil (GER) | 2.5 | ml | Lysine (LYS) | 5 | g |
| Ghana Shea Butter | 275 | g | Methyl Sulfonyl | 25 | g |
| (GSB) | | | Methane (MSM) | | |
| Glycerol (GLY) | 10 | ml | Niacinamide (NCM) | 2.5 | g |
| Ho Wood Oil (HWO) | 2.5 | ml | Oleic Acid (OLA) | 5 | ml |
| Jasmine Oil (JAS) | 2.5 | ml | Praline (PRL) | 5 | g |
| Kanuka Oil (KNO) | 2.5 | ml | Protocatechuic Acid | 10 | g |
| | | | (PCT) | | |
| Lavandin Oil (LVO) | 10 | ml | Sericin | 50 | g |
| Limonene Oil (LMN) | 40 | ml | Sodium Polyacrylate | 20 | g |
| Litsea Cubeba Oil | 5 | ml | Vitamin A (VTA) | 2.5 | g |
| (LCO) | | | | | |
| Marula Oil (MRL) | 2.5 | ml | Vitamin B3 (VTB3) or | 12.5 | g |
| | | | Niacin | | |
| Nerolina Oil (NRL) | 2.5 | ml | Vitamin C (VTC) | 2.5 | g |
| Black Cumin Seed Oil | 50 | ml | Vitamin E (VTE) | 2.5 | g |
| (BCS) | | | | | |

| | | |
|---|---|---|
| Palmarosa Oil (PLO) | 2.5 | ml |
| Panthenol (PNT) | 10 | ml |
| Rosemary Oil (RMO) | 5 | ml |
| Sea Buckthorn Oil | 5 | ml |
| (SBO) | | |
| Tamanu Oil (TMU) | 5 | ml |

### Example 5

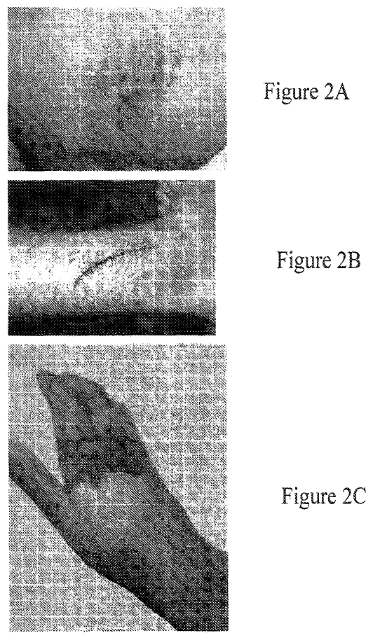

There are many first aid products currently on the market focused on treating abrasions (FIG. 2A), cuts (FIG. 2B), and minor burns (FIG. 2C) that have a variety of effectiveness and varied times to achieve healing compared to no treatment besides cleansing. One of the oldest compositions and most successful in terms of global distribution is a triple antibiotic ointment called NEOSPORIN™ by Johnson & Johnson (1952). Its formulation is very basic including three antibiotics: neomycin, polymyxin, and bacitracin. It also contains petroleum jelly, cocoa butter, cottonseed oil, sodium pyruvate, and tocopheryl acetate. Since many users develop allergies to neomycin, Johnson & Johnson offers a double antibiotic composition containing only bacitracin and polymyxin called POLYSPORIN™. The NEOSPORIN™ composition, while very popular for years, has essentially no ingredients proven to hasten healing. Petroleum jelly from oil compositions is also called Vaseline as branded by Unilever and only prevents moisture loss from the skin. The only potentially healing agent would be cocoa butter that is processed from whole cocoa beans and melts at just under body temperature, making it convenient to use in many compositions, including soaps and lotions. Cocoa butter contains Oleic acid (34.5%-Omega-9, used in food and soap), Stearic acid (34.5%-surfactant, detergents, soaps, lubricants), Palmitic acid (26.0%-soaps, food compositions, cosmetics), Linoleic acid (3.2%-Omega-6, essential oil, surfactant, anti-inflammatory), Arachidic acid (1.0%-detergents, lubricants) and Palmitoleic acid (0.3%-Omega-7). Cottonseed oil is a cooking oil. Sodium pyruvate protects against peroxides. Tocopherol acetate is a form of Vitamin E that can resist acid destruction. None of these NEOSPORIN™ components are able to hasten healing in the skin.

Establishing formulations for these problems is complicated by utilizing many of the components collected due to their long history of enabling accelerated healing. The disclosed composition has been used by nearly a dozen people who had received abrasions, cuts, and minor burns at different times. The results of their use of the composition show a consistent ability to heal these skin wounds rapidly, usually within 2 to 7 days depending upon the size of the wound. Small wounds or injuries usually are sealed and healed within 2-4 days. The larger abrasions on extremities heal well with little evidence of scarring within 7-9 days that would normally take 10 to 14 days. So these results demonstrate a composition that clearly improves healing in a short time scale with little scarring after it is healed. There also is a clear protection
against microbial contamination and infection at the same time. In fact, one can simply apply the composition to an infected finger nail or toe nail that had that had been previously cut or injured that over a few days will show the infection has been eliminated while the composition continues to hasten healing.

### Example 6

A 39 year white male was a bicycle rider going on trails in the area. He fell while riding a trail and landed on his left side inducing a large, 5 by 12 cm skin abrasion with bleeding. On return from the ride, he washed it off and placed NEOSPORIN™ on the wound. The next day, he was offered to replace the NEOSPORIN™ with the disclosed composition. His experience with NEOSPORIN™ for this kind of partial thickness skin wound, would take 10 to 14 days for it to heal. The three times a day usage of this product permitted the healing to be essentially over in 5-6 days with little evidence of scarring.

### Example 7

A 70 year old white male fell at home while working outside and suffered a superficial laceration of the skin on the knee. The disclosed composition was applied to the injury after cleaning. The disclosed composition was applied three times a day. By the second day, the wound was closed and no longer weeping fluid and had lost most of its pain. By the fourth day, the wound was essentially healed over and without pain or sign of infection.

### Example 8

A 52 year old white male applied the disclosed composition on multiple occasions of scratches, scrapes, and cuts. The disclosed composition treats injuries without infection, and healed in half the time usually required for NEOSPORIN™

Table 6 lists the composition formulation for treatment of cuts, abrasions, and minor burns.

TABLE 6
Abrasions, Cuts, and Minor Bums Composition Formulation

| Hydrophobic | | | Hydrophilic | | |
|---|---|---|---|---|---|
| Components | amount | | Components | amount | |
| a-Pinene (APN) | 60 | ml | 1-Tetradecanol | 2.5 | g |
| Acai Berry | 10 | ml | AF (Artie Fish) | 40 | g |
| | | | Collagen (AFC) | | |
| Anethole Anise | 5 | ml | Agave nectar (AGV) | 10 | ml |
| Camphor | | | | | |
| Argan Nut Oil (AGN) | 5 | ml | Alanine (ALA) | 5 | g |
| Baobab Oil (BAB) | 5 | ml | Aloe Vera Leaf Gel | 275 | g |
| | | | (AVLG) | | |
| Bees Wax (BW) | 40 | g | Arginine (ARG) | 5 | g |
| Benzoin (BZN) | 4 | ml | Distilled water | 500 | ml |
| Benzyl Alcohol (BA) | 50 | ml | Ferulic Acid (FRA) | 10 | g |
| Cabreuva Oil | 5 | ml | Glutamine (GLU) | 5 | g |
| Calendula Oil (CDA) | 5 | ml | Glycine (GLY) | 5 | g |
| Camellia Oil (CMA) | 10 | ml | Histidine (HST) | 5 | g |
| Capaiba Oil (CPO) | 10 | ml | Hydroxyproline (HDP) | 5 | g |
| Caprylic acid (CCT) | 10 | ml | Isoleucine (ISL) | 5 | g |
| Carvacrol (CVC) | 5 | ml | Lecithin (LCT) | 25 | g |
| Caryophyllene (CAP) | 10 | ml | Leucine (LUC) | 5 | g |
| Cetyl Alcohol (CTA) | 40 | g | Linoleic Acid (LOA) | 5 | ml |
| Chamomile German | 10 | ml | Lysine (LYS) | 5 | g |
| Oil (CGB) | | | | | |
| Ghana Shea Butter | 275 | g | Methyl Sulfonyl | 25 | g |
| (GSB) | | | Methane (MSM) | | |
| Glycerol (GLY) | 10 | ml | Niacinamide (NCM) | 2.5 | g |
| Kaempferol | 5 | g | Oleic Acid (OLA) | 5 | ml |
| Kanuka Oil (KNO) | 2.5 | ml | Praline (PRL) | 5 | g |
| Limonene Oil (LMN) | 40 | ml | Protocatechuic Acid | 10 | g |
| | | | (PCT) | | |
| Litsea Cubeba Oil | 5 | ml | Sericin | 50 | g |
| (LCO) | | | | | |
| Myrrh | 5 | ml | Sodium Polyacrylate | 20 | g |
| Nerolina Oil (NRL) | 2.5 | ml | Vitamin A (VTA) | 2.5 | g |
| Niaouli Oil | 5 | ml | Vitamin B3 (VTB3) or | 12.5 | g |
| | | | Niacin | | |
| Black Cumin Seed | 50 | ml | Vitamin C (VTC) | 2.5 | g |
| Oil (BCS) | | | | | |
| Panthenol (PNT) | 10 | ml | Vitamin E (VTE) | 2.5 | g |

| | | |
|---|---|---|
| Peppermint | 5 | ml |
| Rosemary Oil (RMO) | 5 | ml |
| Sachi Inchi oil | 5 | ml |
| Sea Buckthom Oil | 5 | ml |
| (SBO) | | |
| Tamanu Oil (TMU) | 5 | ml |
| Terpineol | 5 | ml |
| Thyme Red, White | 5 | ml |

### Example 9

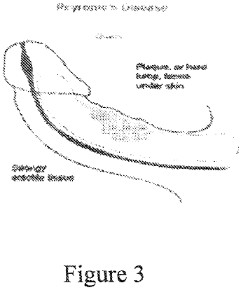

Peyronie's Disease is a connective tissue disorder of the penis affecting 5% of the male population. It involves fibrous plaques developing along the fibrous sheath, the tunica albuginea, that surrounds the spongy erectile tissue, the corpus cavernosa, in the penis. When the penis fills with blood and erects, these fibrous plaques or bands prevent that portion of the penis from erecting properly. This leads to two types of deformities of the penis: the more common is a marked curvature of the penis in any direction at the point of the stricture (FIG. 3) while the least common is an hour glass stricture completely around the penis. Regardless of the type of stricture, as it progresses it causes more deformity. Penile penetration during intercourse becomes painful and eventually impossible. A small percentage of men with Dupuytren's Disease or Contracture also develop Peyronie's Disease for unknown reasons. There is no known cause of these progressive deformities of the penis. Without treatment, 10-15% will spontaneously improve over time, 40-50% will get worse, and 35-50% will not change.

Currently, there is no effective treatment that does not include a high risk of doing more harm. Injection of collagenase into the plaques can soften them to reduce the strictures causing the deformities. However, if the physician happens to inject collagenase into the spongy erectile tissue, it can be weakened and fracture during intercourse producing a non-functional penis.

Trying to surgically remove these plaques has a high risk of permanently injuring the penis, which can lead to internal fracture leading to non-function regarding intercourse. At this point, there is no effective treatment to this disease without a high risk of making it worse.

Due to the observations of the effect of the disclosed composition in people with Dupuytren's Disease and Contracture, a similar preparation was tried in men with untreated Peyronie's disease. Four patients were selected and provided with the topical composition to apply twice a day to the region of the penis involved. At one month follow up, the first two patients, one with a marked lateral curve and the other with an hour-glass deformity near the end of the penis returned quite pleased with their results. The marked curve patient's penis had the curvature reduced by 50% at that time permitting him to again have intercourse. The second patient with the hour-glass deformity had his stricture essentially resolve in this time leaving him to have fairly normal intercourse as well. They are continuing to use the medication. The second two patients are in treatment currently and will be returning with their one month follow up soon.

### Example 10

A 34 year old, middle eastern male with Peyronie's disease of the cicatrix, circumferential type with significant curvature of the penis restricting intercourse. Photos and measurements were made after injection of agents into the base of the penis to cause a maximal erection of the penis. The patient was given the disclosed composition for treatment of Peyronie's Disease and told to apply it twice a day to the lesion near by tissue in the penis. On 3 month follow up, the penile constricting lesion had significantly loosened with a resulting reduction of the curvature from 80 degrees down to 45 degrees which has been sufficient to permit intercourse. Additional treatment and follow up are underway.

### Example 11

A 38 year old, middle eastern male with Peyronie's disease of the dorsal ligament as a thickened nodule causing a dorsal and partially lateral curvature of the penis with significant curvature of the penis restricting intercourse. Photos and measurements were made after injection of agents into the base of the penis to cause a maximal erection of the penis. The patient was given the disclosed composition for treatment of Peyronie's Disease and told to apply it twice a day to the lesion near by tissue in the penis. After 4 months of follow up, the dorsal penile ligament lesion had softened permitting the curvature to reduce from 65 degrees down to 35 degrees permitting the ability to have intercourse again. Treatment continues with additional observation periods in follow up.

Table 7 lists the composition formulation for treatment of Peyronie's Disease or Induration Penis Plastica.

TABLE 7
Peyronie's Disease or Induration
Penis Plastica Composition Formulation

| Hydrophobic | | | Hydrophilic | | |
|---|---|---|---|---|---|
| Components | amount | | Components | amount | |
| a-Pinene (APN) | 60 | ml | 1-Tetradecanol | 2.5 | g |
| Acai Berry | 10 | ml | AF (Artie Fish) | 40 | g |
| | | | Collagen (AFC) | | |
| Anethole Anise | 5 | ml | Agave nectar (AGV) | 10 | ml |
| Camphor | | | | | |
| Argan Nut Oil (AGN) | 5 | ml | Alanine (ALA) | 5 | g |
| Bees Wax (BW) | 40 | g | Aloe Vera Leaf Gel | 275 | g |
| | | | (AVLG) | | |
| Benzoin (BZN) | 4 | ml | Arginine (ARG) | 5 | g |
| Benzyl Alcohol (BA) | 50 | ml | Distilled water | 500 | ml |
| Calendula Oil (CDA) | 5 | ml | Ferulic Acid (FRA) | 10 | g |
| Camellia Oil (CMA) | 10 | ml | Glutamine (GLU) | 5 | g |
| Capaiba Oil (CPO) | 10 | ml | Glycine (GLY) | 5 | g |
| Caprylic, Capric | 10 | ml | Histidine (HST) | 5 | g |
| Triglyceride (CCT) | | | | | |
| Carvacrol (CVC) | 5 | ml | Hydroxyproline (HDP) | 5 | g |
| Caryophyllene (CAP) | 10 | ml | Isoleucine (ISL) | 5 | g |
| Cetyl Alcohol (CTA) | 40 | g | Lecithin (LCT) | 25 | g |
| Chamomile German Oil | 10 | ml | Leucine (LUC) | 5 | g |
| (CGB) | | | | | |
| Elemi Oil (EMO) | 5 | ml | Linoleic Acid (LOA) | 5 | ml |
| Ghana Shea Butter | 275 | g | Lysine (LYS) | 5 | g |
| (GSB) | | | | | |
| Glycerol (GLY) | 10 | ml | Methyl Sulfonyl | 25 | g |
| | | | Methane (MSM) | | |
| Kanuka Oil (KNO) | 2.5 | ml | Niacinamide (NCM) | 2.5 | g |
| Limonene Oil (LMN) | 40 | ml | Oleic Acid (OLA) | 5 | ml |
| Litsea Cubeba Oil | 5 | ml | Praline (PRL) | 5 | g |
| (LCO) | | | | | |
| Marula Oil (MRL) | 2.5 | ml | Protocatechuic Acid | 10 | g |
| | | | (PCT) | | |
| Black Cumin Seed Oil | 50 | ml | Sericin | 50 | g |
| (BCS) | | | | | |
| Palo Santo | 2.5 | ml | Sodium Polyacrylate | 20 | g |
| Panthenol (PNT) | 10 | ml | Vitamin A (VTA) | 2.5 | g |
| Rosemary Oil (RMO) | 5 | ml | Vitamin B3 (VTB3) or | 12.5 | g |
| | | | Niacin | | |
| Sea Buckthorn Oil | 5 | ml | Vitamin C (VTC) | 2.5 | g |
| (SBO) | | | | | |
| Terpineol | 5 | ml | Vitamin E (VTE) | 2.5 | g |

### Example 12

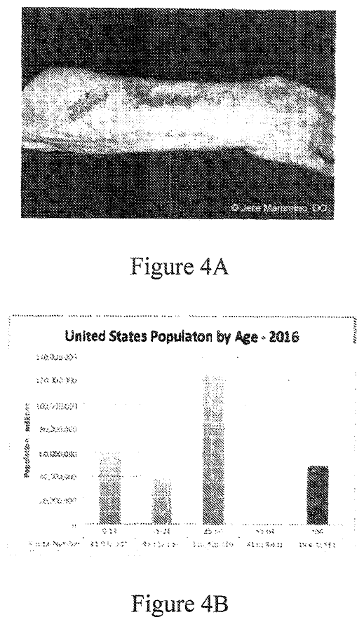

As everyone ages overtime, their skin is under easy observation, and readily shows multiple aging changes. The age relating skin changes include thinning of the outer surface of the skin, loss of substance and softness in both the outer and deeper skin components, wrinkles, easy bruising, appearance of pigmented flat lesions, scaly lesions, skin tags, loss of hair, and scratches which take much longer to heal (FIG. 4A). The appearance and the rate of the increasing incidence of these aging disorders predominantly depend upon the individual's history

of sun exposure, history and the degree of routine skin care, race, as well as, genetics. An increasing incidence of skin cancers also will occur with increasing age. Routine skin care of the face, as well as, the use of sun protectants is more common in both men and women than routine skin care of the hands, arms, legs, and feet. Thus, it is very common to see multiple types of accelerated skin aging problems in the arms and hands of the vast majority of the aging populations.

If one examines the USA population by age from 2016, one finds there are 49.4 million people over the age of 64 years of age (FIG. 4B). This is 15.2% of the population that is subject to aging skin and would certainly have the skin aging readily observable and open for potential treatment. Of this age group, 44.3% are males and 55.7% are females. While aging skin can certainly begin in the 55 year olds, it is not as frequent as in the >64 year olds.

### Example 13

A 72 year old, white male, had progressive hair loss on the dorsal surfaces of both forearms and hands that had progressed over the last several years. Skin thinning had also progressed to the marked reduction of surface hair and easy bruising of the skin. Several superficial skin tears had also occurred leaving scars.

There were many actinic keratoses present on these same surfaces from previous sun damage. This person had been using moisturizers for several years on these surfaces without any effect on delaying these-age related changes. The disclosed composition was applied to the dorsal surface of the right forearm while moisturizers were continued on the left forearm. Within a couple of months, there was noticeable difference in the appearance of the two forearms. The left forearm was unchanged.

The right forearm skin had begun to thicken with increased terger of the skin noted along with the epidermal layer clearly thickening. The actinic keratoses began to flatten and become thinner and smoother. Then, new hair began to appear across the right forearm without any new hair forming on the left forearm. The test subject was allowed to apply the disclosed composition to both forearms after the changes became quite obvious when examining both forearms. Several months of continued use of the disclosed composition on both forearms shows established hair, both dark and white, growing well to its normal length, increased dermal thickening, strengthening and thickening of the epidermis without any new tears, and ongoing reduction in the actinic keratoses size and appearance.

Table 8 lists the composition formulation for treatment of exposed aging skin.

TABLE 8
Old-Age Skin Treatment Composition Formulation

| Hydrophobic | | | Hydrophilic | | |
|---|---|---|---|---|---|
| Components | amount | | Components | amount | |
| a-Pinene (APN) | 60 | ml | 1-Tetradecanol | 2.5 | g |
| Acai Berry | 10 | ml | AF (Artie Fish) | 140 | g |
| | | | Collagen (AFC) | | |
| Argan Nut Oil (AGN) | 5 | ml | Agave nectar (AGV) | 10 | ml |
| Baobab Oil (BAB) | 5 | ml | Alanine (ALA) | 5 | g |
| Bees Wax (BW) | 40 | g | Aloe Vera Leaf Gel | 275 | g |
| | | | (AVLG) | | |
| Benzoin (BZN) | 4 | ml | Arginine (ARG) | 5 | g |
| Benzyl Alcohol (BA) | 50 | ml | Distilled water | 500 | ml |
| Cabreuva Oil | 5 | ml | Ferulic Acid (FRA) | 10 | g |
| Caffeic acid | 5 | g | Glutamine (GLU) | 5 | g |
| Calendula Oil (CDA) | 5 | ml | Glycine (GLY) | 5 | g |
| Camellia Oil (CMA) | 10 | ml | Histidine (HST) | 5 | g |
| Capaiba Oil (CPO) | 10 | ml | Hydroxyproline (HDP) | 5 | g |
| Caprylic, Capric | 10 | ml | Isoleucine (ISL) | 5 | g |
| Triglyceride (CCT) | | | | | |
| Carvacrol (CVC) | 5 | ml | Lecithin (LCT) | 25 | g |
| Caryophyllene (CAP) | 10 | ml | Leucine (LUC) | 5 | g |
| Cetyl Alcohol (CTA) | 40 | g | Linoleic Acid (LOA) | 5 | ml |
| Chamomile German | 10 | ml | Lysine (LYS) | 5 | g |
| Oil (CGB) | | | | | |
| Elemi Oil (EMO) | 5 | ml | Methyl Sulfonyl Methane | 25 | g |
| | | | (MSM) | | |
| Geranium Oil (GER) | 2.5 | ml | Niacinamide (NCM) | 2.5 | g |
| Ghana Shea Butter | 275 | g | Oleic Acid (OLA) | 5 | ml |
| (GSB) | | | | | |
| Glycerol (GLY) | 10 | ml | Praline (PRL) | 5 | g |
| Ho Wood Oil (HWO) | 2.5 | ml | Protocatechuic Acid | 10 | g |
| | | | (PCT) | | |
| Jasmine Oil (JAS) | 2.5 | ml | Sericin | 150 | g |
| Kaempferol | 5 | g | Sodium Polyacrylate | 20 | g |
| Kanuka Oil (KNO) | 2.5 | ml | Valine (VAL) | 5 | g |
| Lavandin Oil (LVO) | 10 | ml | Vitamin A (VTA) | 2.5 | g |
| Limonene Oil (LMN) | 40 | ml | Vitamin B3 (VTB3) or | 12.5 | g |
| | | | Niacin | | |
| Litsea Cubeba Oil | 5 | ml | Vitamin C (VTC) | 2.5 | g |
| (LCO) | | | | | |
| Marula Oil (MRL) | 2.5 | ml | Vitamin E (VTE) | 2.5 | g |

| | | |
|---|---|---|
| Myrrh | 5 | ml |
| Nerolina Oil (NRL) | 2.5 | ml |
| Niaouli Oil | 5 | ml |
| Black Cumin Seed | 50 | ml |
| Oil (BCS) | | |
| Palmarosa Oil (PLO) | 2.5 | ml |
| Palo Santo | 2.5 | ml |
| Panthenol (PNT) | 10 | ml |
| Peppermint | 5 | ml |
| Rose Hip Seed oil | 5 | ml |
| Rosemary Oil (RMO) | 5 | ml |
| Sachi Inchi oil | 5 | ml |
| Sea Buckthorn Oil | 5 | ml |
| (SBO) | | |
| Tamanu Oil (TMU) | 5 | ml |
| Vetiver | 2.5 | ml |

### Example 14

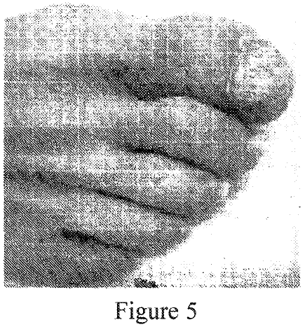

Toe nail fungus is medically known as onychomycosis, or tinea unguium, commonly affects toe nails and can be progressive to the state of nail destruction, if not treated properly (FIG. 5). It can also affect the finger nails. It is the most common of all the nail afflictions affecting 10% of the population and represents 50% of all nail afflictions. The progression is from mild forms to the most severe with loss of most of the normal nail.

The pathogens involved in onchyomycosis are all represented in the fungus kingdom that includes dermatophytes in the western world and Candida and non-dermatophyes in the tropics and subtropics. The dermatophytes most common representative is Trichophyton rubrum with multiple related species. Candida species predominantly affect the finger nails, rather than the toe nails. The non-dermatophytes are predominantly molds, mostly from the genus of Neoscytalidium, Scopulariopsis, and Aspergillus. Conditions that can be confused with onychomycosis include nail psoriasis, lichen planus, contact dermatitis, and nail bed tumors such as melanoma, thrauma, and Q yellow nail syndrome.

Six patients with this condition have used the disclosed composition for treating infected toe nails. One patient had the most involvement with all ten toe nails involved to the severe stage. His podiatrist suggested he needed to have all ten nails removed to enable healing his problem. He was supplied with the disclosed formulation with application once a day after showering, as well as, keeping his nails trimmed well through out the treatment. Within one month, most all of the nails were growing out with normal nails below the fungal infected outer edge. By two to three months, all of the toe nails had been eliminated from their fungus. The other patients had involvements of lesser degree, but all patients had eliminated their toe nail fungus in a similar time frame.

### Example 15

A 66 year old white male had chronic, severe onychomycosis involving fungal infections of all of his toe nails and was being followed by a podiatrist. After trying several known potential remedies over months to a few years, the podiatrist told this patient that there was nothing else topically to help his problem. He asked to schedule him for removal of all of his toenails so he could remove the difficult to heal infected toenails and clear the growth beds that were not responding to his treatment. He delayed the proposed surgery and started applying the disclosed composition daily to his nails after showering. Before starting the treatment, the podiatrist cleaned the extra debris from the infection and cut the nails close. Within a few weeks, the toenails began to heal and growing out healthy nails from the nail beds. When all of the infected toenail and base had been cleared, he had normal looking nails and no longer needed the surgery the podiatrist wanted to perform.

### Example 16

A 69 year old white male had developed chronic onychomycosis, or toe nail fungus, involving most of the toe nails on both feet, but with different degrees of infection. The disclosed composition was applied once a day on all of the nails after shower or bath. The nails were to be kept well clipped and the disclosed composition applied daily. Over a few weeks, the mildly infected nails cleared of their fungus, but the more heavily infected nails took a few more weeks to clear. But, at the end, all of the nails recovered from their fungal infection.

Table 9 lists the composition formulation for treatment of toe nail fungus.

TABLE 9
Toe Nail Fungus Composition Formulation

| Hydrophobic | | | Hydrophilic | | |
|---|---|---|---|---|---|
| Components | amount | | Components | amount | |
| a-Pinene (APN) | 60 | ml | 1-Tetradecanol | 2.5 | g |
| Acai Berry | 10 | ml | AF (Artie Fish) | 40 | g |
| | | | Collagen (AFC) | | |
| Anethole Anise | 5 | ml | Agave nectar (AGV) | 10 | ml |
| Camphor | | | | | |
| Argan Nut Oil (AGN) | 5 | ml | Alanine (ALA) | 5 | g |
| Baobab Oil (BAB) | 5 | ml | Aloe Vera Leaf Gel | 275 | g |
| | | | (AVLG) | | |
| Bees Wax (BW) | 40 | g | Arginine (ARG) | 5 | g |
| Benzoin (BZN) | 4 | ml | Distilled water | 500 | ml |
| Benzyl Alcohol (BA) | 50 | ml | Ferulic Acid (FRA) | 10 | g |
| Cabreuva Oil | 5 | ml | Glutamine (GLU) | 5 | g |
| Calendula Oil (CDA) | 5 | ml | Glycine (GLY) | 5 | g |
| Camellia Oil (CMA) | 10 | ml | Histidine (HST) | 5 | g |
| Capaiba Oil (CPO) | 10 | ml | Hydroxyproline (HDP) | 5 | g |
| Caprylic acid (CCT) | 10 | ml | Isoleucine (ISL) | 5 | g |
| Carvacrol (CVC) | 5 | ml | Lecithin (LCT) | 25 | g |
| Cetyl Alcohol (CTA) | 40 | g | Leucine (LUC) | 5 | g |
| Chamomile German | 10 | ml | Linoleic Acid (LOA) | 5 | ml |
| Oil (CGB) | | | | | |
| Ghana Shea Butter | 275 | g | Lysine (LYS) | 5 | g |
| (GSB) | | | | | |
| Glycerol (GLY) | 10 | ml | Methyl Sulfonyl | 25 | gm |
| | | | Methane (MSM) | | |
| Kanuka Oil (KNO) | 2.5 | ml | Niacinamide (NCM) | 2.5 | g |
| Limonene Oil (LMN) | 40 | ml | Oleic Acid (OLA) | 5 | ml |
| Litsea Cubeba Oil | 5 | ml | Praline (PRL) | 5 | g |
| (LCO) | | | | | |
| Myrrh | 5 | ml | Protocatechuic Acid | 10 | g |
| | | | (PCT) | | |
| Niaouli Oil | 5 | ml | Sericin | 50 | g |
| Black Cumin Seed Oil | 50 | ml | Sodium Polyacrylate | 20 | g |
| (BCS) | | | | | |
| Panthenol (PNT) | 10 | ml | Vitamin A (VTA) | 2.5 | g |
| Peppermint | 5 | ml | Vitamin B3 (VTB3) or | 12.5 | g |
| | | | Niacin | | |
| Sea Buckthorn Oil | 5 | ml | Vitamin C (VTC) | 2.5 | g |
| (SBO) | | | | | |
| Terpineol | 5 | ml | Vitamin E (VTE) | 2.5 | g |

| | | |
|---|---|---|
| Thyme Red, White | 5 | ml |
| Winter Savory | 5 | ml |

### Example 17

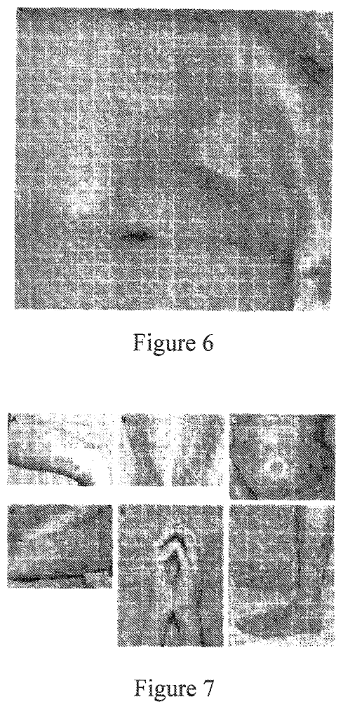

Aphthous ulcers or stomatitis are characterized by repeated, benign and non-contagious oral cavity ulcerations that are self-limited to usually 7-10 days, as shown in FIG. 6. These are painful and sensitive to increased pain by topical exposure to compositions with excessive pH, spices, and increased temperature. Episodes usually occur 3-6 times a year for several years around the time of adolescence. There is no known cause but are thought to be multi-factorial.

While the ulceration triggers are not identified, the ulcers develop due to immune cell reactions from T-cells, mast cells, and macrophages to unknown triggers with the emphasis on T-cell involvement. Forty percent of persons getting these lesions have a family history of involvement. Some episodes seem to clearly be responding to acute, excessive stress in those who get these ulcers, but most do not. The natural duration of 7-10 days remains fairly uniform, even in spite of several different types of proposed treatments.

The disclosed composition has demonstrated the ability to reduce the duration of these ulcers to 2-4 days while relieving the pain. However, it does not appear to prevent the regular occurrence of these ulcers. Approximately 10 patients have used this formulation and uniformly experience a shorter duration of time and a reduction of pain.

### Example 18

A 65 year old white male, was having occasional episodes of aphthous ulcers of the mouth that were typical in severity and duration with pain lasting 7 to IO days and final clearing from 2 to 2.5 weeks. These aphthous ulcers were occurring every three to four months without an obvious cause. The disclosed composition was applied at the start of an episode. The disclosed composition was designed to be thick enough to stay within the bed of the ulcer with application 2 to 4 times a day. From starting the use of the disclosed composition on the first day of the lesion appearing, and treating it 3 times a day after meals and again at bed time, the ulcer lost its pain in 2-3 days and disappeared in 4-5 days. Due to benzocaine being used as a product ingredient, the ulcer had a significant decrease in pain during the treatment phase and prior to its healing.

Table 10 lists the composition formulation for treatment of aphthous ulcers of the mouth.

TABLE 10
Aphthous Ulcers of the Mouth Composition Formulation

| Hydrophobic | | | Hydrophilic | | |
|---|---|---|---|---|---|
| Components | amount | | Components | amount | |
| a-Pinene (APN) | 60 | ml | 1-Tetradecanol | 2.5 | g |
| Acai Berry | 10 | ml | AF (Artie Fish) | 40 | g |
| | | | Collagen (AFC) | | |
| Anethole Anise | 5 | ml | Agave nectar (AGV) | 10 | ml |
| Camphor | | | | | |
| Argan Nut Oil (AGN) | 5 | ml | Alanine (ALA) | 5 | g |
| Baobab Oil (BAB) | 5 | ml | Aloe Vera Leaf Gel | 275 | g |
| | | | (AVLG) | | |
| Bees Wax (BW) | 40 | g | Arginine (ARG) | 5 | g |
| Benzoin (BZN) | 4 | ml | Benzocaine | 3 | g |
| Benzyl Alcohol (BA) | 50 | ml | Distilled water | 500 | ml |
| Cabreuva Oil | 5 | ml | Ferulic Acid (FRA) | 10 | g |
| Caffeic acid | 5 | g | Glutamine (GLU) | 5 | g |
| Calendula Oil (CDA) | 5 | ml | Glycine (GLY) | 5 | g |
| Capaiba Oil (CPO) | 10 | ml | Histidine (HST) | 5 | g |
| Caprylic acid (CCT) | 10 | ml | Hydroxyproline (HDP) | 5 | g |
| Carvacrol (CVC) | 5 | ml | Isoleucine (ISL) | 5 | g |
| Caryophyllene (CAP) | 10 | ml | Lecithin (LCT) | 25 | g |
| Cedarwood essential Oil | 2.5 | nl | Leucine (LUC) | 5 | g |
| Cetyl Alcohol (CTA) | 40 | g | Linoleic Acid (LOA) | 5 | ml |
| Chamomile German | 10 | ml | Lysine (LYS) | 5 | g |
| Oil (CGB) | | | | | |
| Ghana Shea Butter | 275 | g | Methyl Sulfonyl | 25 | g |
| (GSB) | | | Methane (MSM) | | |
| Glycerol (GLY) | 10 | ml | Niacinamide (NCM) | 2.5 | g |
| Kanuka Oil (KNO) | 2.5 | ml | Oleic Acid (OLA) | 5 | ml |
| Limonene Oil (LMN) | 40 | ml | Praline (PRL) | 5 | g |
| Litsea Cubeba Oil | 5 | ml | Protocatechuic Acid | 10 | g |
| (LCO) | | | (PCT) | | |
| Niaouli Oil | 5 | ml | Sericin | 50 | g |
| Black Cumin Seed Oil | 50 | ml | Sodium Polyacrylate | 20 | g |
| (BCS) | | | | | |
| Panthenol (PNT) | 10 | ml | Vitamin A (VTA) | 2.5 | g |
| Peppermint | 5 | ml | Vitamin B3 (VTB3) or | 12.5 | g |
| | | | Niacin | | |
| Sea Buckthorn Oil | 5 | ml | Vitamin C (VTC) | 2.5 | g |
| (SBO) | | | | | |
| Thyme Red, White | 5 | ml | Vitamin E (VTE) | 2.5 | g |

| | | |
|---|---|---|
| Winter Savory | 5 | ml |

### Example 19

Scleroderma is a systemic disease that results in progressive fibrosis of organs starting with skin lesions that can then progress to involving major organs, such as, the heart, lungs, intestines and kidneys. The pathophysiology for the skin appears to involve the laying down in a random orientation of collagen much like scar formation diffusively in the subcutaneous tissues. This leads to joint immobilization starting in the fingers and spreading into the wrists and elbows as well as in the toes and ankle (FIG. 7). Its increased pressure beneath the skin also reduces blood flow causing extensive Raynaud's phenomenon, or skin blanching episodes on the digits that readily leads to poorly healing ulcerations and potential digit loss.

A white female, 52 years old developed Scleroderma with cutaneous manifestations that resulted in contractures of all the joints of the fingers on both hands, both wrists, and early limitations of both elbows. Fibrosis of the fingers only left about 5-10% motion in all fingers. The wrist joints were about 50% inhibited with elbows about 30% inhibited, all due to the subcutaneous fibrosis. She had evidence of some areas of collagen formation beneath the skin in both upper extremities. The patient's primary complaint was that her skin was giving her so many symptoms on a 24 hour basis; she was treating subcutaneous discomfort in both arms with over the counter skin moisturizers every two hours without much relief, including at night. She tested the disclosed composition on the skin of both arms and found symptomatic relief. Instead of every two hours, she found by applying this product only every 12 hours, she got improved relief of her previous cutaneous symptoms. In addition, she had developed significant Raynaud's Phenomenon on fingers on both hands with early joint ulcerations noted on several fingers. With some early systemic symptoms, she was treated with an oral drug for Scleroderma for several months but systemic disease progression continued. She underwent bone marrow ablation and received a bone marrow transplant and is currently under remission. The disclosed composition was discontinued when her disease progression began to advance to systemic symptoms.

Table 11 lists the composition formulation for treatment of cutaneous scleroderma.

TABLE 11
Cutaneous Scleroderma Composition Formulation

| Hydrophobic | | | Hydrophilic | | |
|---|---|---|---|---|---|
| Components | amount | | Components | amount | |
| a-Pinene (APN) | 60 | ml | 1-Tetradecanol | 2.5 | g |
| Acai Berry | 10 | ml | AF (Artie Fish) | 140 | g |
| | | | Collagen (AFC) | | |
| Argan Nut Oil (AGN) | 5 | ml | Agave nectar (AGV) | 10 | ml |
| Baobab Oil (BAB) | 5 | ml | Alanine (ALA) | 5 | g |
| Bees Wax (BW) | 40 | g | Aloe Vera Leaf Gel | 275 | g |
| | | | (AVLG) | | |
| Benzoin (BZN) | 4 | ml | Arginine (ARG) | 5 | g |
| Benzyl Alcohol (BA) | 50 | ml | Distilled water | 500 | ml |
| Calendula Oil (CDA) | 5 | ml | Ferulic Acid (FRA) | 10 | g |
| Camellia Oil (CMA) | 10 | ml | Glutamine (GLU) | 5 | g |
| Capaiba Oil (CPO) | 10 | ml | Glycine (GLY) | 5 | g |
| Caprylic, Capric | 10 | ml | Histidine (HST) | 5 | g |
| Triglyceride (CCT) | | | | | |
| Carvacrol (CVC) | 5 | ml | Hydroxyproline (HDP) | 5 | g |
| Caryophyllene (CAP) | 10 | ml | Isoleucine (ISL) | 5 | g |
| Cetyl Alcohol (CTA) | 40 | g | Lecithin (LCT) | 25 | g |
| Chamomile German | 10 | ml | Leucine (LUC) | 5 | g |
| Oil (CGB) | | | | | |
| Elemi Oil (EMO) | 5 | ml | Linoleic Acid (LOA) | 5 | ml |
| Geranium Oil (GER) | 2.5 | ml | Lysine (LYS) | 5 | g |
| Ghana Shea Butter | 275 | g | Methyl Sulfonyl Methane | 25 | g |
| (GSB) | | | (MSM) | | |
| Glycerol (GLY) | 10 | ml | Niacinamide (NCM) | 2.5 | g |
| Ho Wood Oil (HWO) | 2.5 | ml | Oleic Acid (OLA) | 5 | ml |
| Jasmine Oil (JAS) | 2.5 | ml | Praline (PRL) | 5 | g |
| Kanuka Oil (KNO) | 2.5 | ml | Protocatechuic Acid | 10 | g |
| | | | (PCT) | | |
| Lavandin Oil (LVO) | 10 | ml | Sericin | 50 | g |
| Limonene Oil (LMN) | 40 | ml | Sodium Polyacrylate | 20 | g |
| Litsea Cubeba Oil | 5 | ml | Vitamin A (VTA) | 2.5 | g |
| (LCO) | | | | | |
| Marula Oil (MRL) | 2.5 | ml | Vitamin B3 (VTB3) or | 12.5 | g |
| | | | Niacin | | |
| Nerolina Oil (NRL) | 2.5 | ml | Vitamin C (VTC) | 2.5 | g |
| Black Cumin Seed | 50 | ml | Vitamin E (VTE) | 2.5 | g |
| Oil (BCS) | | | | | |

| | | |
|---|---|---|
| Palo Santo | 2.5 | ml |
| Panthenol (PNT) | 10 | ml |
| Rose Hip Seed Oil | 10 | ml |
| Rosemary Oil (RMO) | 5 | ml |
| Sachi Inchi oil | 5 | ml |
| Sea Buckthorn Oil | 5 | ml |
| (SBO) | | |
| Tamanu Oil (TMU) | 5 | ml |

### Example 20

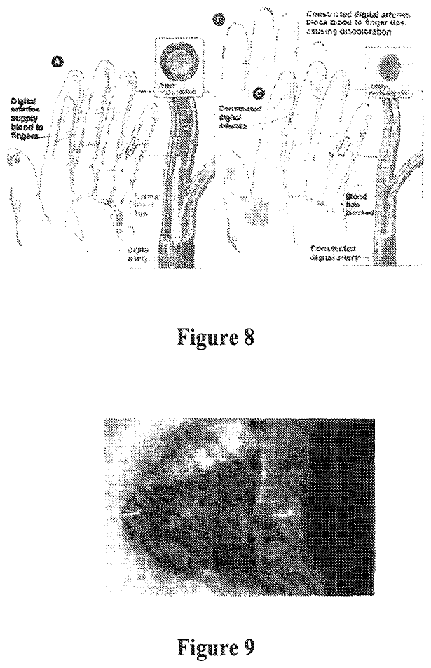

Raynaud's Phenomenon or Syndrome is a medical condition that involves episodic spasm of the arteries that markedly reduces blood flow to the hands and/or feet that can eventually lead to poorly healing ulcers and to gangrene with loss of digits (FIG. 8). The episodes affecting

the hands or feet initially turn white and later blue, often with associated numbness or pain. As the spasm ends with blood flow returning, the affected parts turn red and are typically painful. These episodes usually only last a few minutes, but can last for hours. There are two major types of Raynaud's Phenomenon that includes 4% of the population: primary without a known cause and secondary which occurs related to other primary conditions. The secondary conditions that can cause these episodes include immune disorders, such as, lupus and scleroderma, former trauma including frost bite, smoking, thyroid problems, and certain medications, such as, birth control pills. The existing primary treatment is to eliminate known stimulants, such as, smoking and to avoid exposing the affected extremities to cold temperatures. If these are not effective, then calcium channel blockers or a vasodilator can be tried. There currently are no other known remedies.

The patient that presented with scleroderma also suffered from Raynaud's phenomenon in both hands with sufficient frequency and intensity that multiple ulcers formed on the dorsum of several digits. The disclosed composition for treatment of Raynaud's Phenomenon was applied to the hands, between episodes of Raynaud's attacks, of the patient with scleroderma and improved the color of the fingers which also were found by palpation to be warmer.

Table 12 lists the composition formulation for treatment of Raynaud's Phenomenon.

TABLE 12
Raynaud's Phenomenon Composition Formulation

| Hydrophobic | | | Hydrophilic | | |
|---|---|---|---|---|---|
| Components | amount | | Components | amount | |
| a-Pinene (APN) | 60 | ml | 1-Tetradecanol | 2.5 | g |
| Acai Berry | 10 | ml | AF (Artie Fish) | 40 | g |
| | | | Collagen (AFC) | | |
| Argan Nut Oil (AGN) | 5 | ml | Agave nectar (AGV) | 10 | ml |
| Baobab Oil (BAB) | 5 | ml | Alanine (ALA) | 5 | g |
| Bees Wax (BW) | 40 | g | Aloe Vera Leaf Gel | 275 | g |
| | | | (AVLG) | | |
| Benzoin (BZN) | 4 | ml | Arginine (ARG) | 5 | g |
| Benzyl Alcohol (BA) | 50 | ml | Distilled water | 500 | ml |
| Caffeic acid | 5 | g | Ferulic Acid (FRA) | 10 | g |
| Calendula Oil (CDA) | 5 | ml | Glutamine (GLU) | 5 | g |
| Camellia Oil (CMA) | 10 | ml | Glycine (GLY) | 5 | g |
| Capaiba Oil (CPO) | 10 | ml | Histidine (HST) | 5 | g |
| Caprylic, Capric | 10 | ml | Hydroxyproline (HDP) | 5 | g |
| Triglyceride (CCT) | | | | | |
| Carvacrol (CVC) | 5 | ml | Isoleucine (ISL) | 5 | g |
| Cetyl Alcohol (CTA) | 40 | g | Lecithin (LCT) | 25 | g |
| Chamomile German | 10 | ml | Leucine (LUC) | 5 | g |
| Oil (CGB) | | | | | |
| Ghana Shea Butter | 275 | g | Linoleic Acid (LOA) | 5 | ml |
| (GSB) | | | | | |
| Glycerol (GLY) | 10 | ml | Lysine (LYS) | 5 | g |
| Kanuka Oil (KNO) | 2.5 | ml | Methyl Sulfonyl | 25 | g |
| | | | Methane (MSM) | | |
| Limonene Oil (LMN) | 40 | ml | Niacinamide (NCM) | 2.5 | g |
| Litsea Cubeba Oil | 5 | ml | Oleic Acid (OLA) | 5 | ml |
| (LCO) | | | | | |
| Black Cumin Seed | 50 | ml | Praline (PRL) | 5 | g |
| Oil (BCS) | | | | | |
| Panthenol (PNT) | 10 | ml | Protocatechuic Acid | 10 | g |
| | | | (PCT) | | |
| Terpineol | 5 | ml | Sericin | 50 | g |

| | | |
|---|---|---|
| Sodium Polyacrylate | 20 | g |
| Vitamin A (VTA) | 2.5 | g |
| Vitamin B3 (VTB3) or | 12.5 | g |
| Niacin | | |
| Vitamin C (VTC) | 2.5 | g |
| Vitamin E (VTE) | 2.5 | g |

### Example 21

A common problem with both systemic irradiation and chemotherapy is the development of multiple induced oral mucosal ulcers scattered throughout the mouth to the 40-60% patient incidence level (FIG. 9). These are very painful and have no known treatment at the moment. The pathophysiology of these ulcers relates to the fact that the oral mucosal lining cells normally have a rapid turnover and replacement time. Whenever chemotherapy and/or radiation therapy in the body occurs, the turnover replication rate of these oral mucosal lining cells reduces significantly. This means these cells cannot replicate sufficiently fast enough to replace their normal losses resulting in the formation of a mucosa with major gaps in their surface cell coverage.

Currently there is no treatment for this painful consequence of the tumor treatment. The significant factor one has to consider in developing a replacement is that the increased cell replication agent cannot interfere with the treatment of the tumor. This means that the treatment has to remain local within the mouth and cannot be absorbed into the body that is being treated for systemic cancers.

Table 13 lists the composition formulation for treatment of chemotherapy/radiation therapy induced oral mucosal lesions.

TABLE 13
Chemotherapy/Radiation Therapy Induced Oral
Mucosa! Lesions Composition Formulation

| Hydrophobic | | | Hydrophilic | | |
|---|---|---|---|---|---|
| Components | amount | | Components | amount | |
| a-Pinene (APN) | 60 | ml | 1-Tetradecanol | 2.5 | g |
| Acai Berry | 10 | ml | AF (Artie Fish) | 40 | g |
| | | | Collagen (AFC) | | |
| Anethole Anise | 5 | ml | Agave nectar (AGV) | 10 | ml |
| Camphor | | | | | |
| Argan Nut Oil (AGN) | 5 | ml | Alanine (ALA) | 5 | g |
| Baobab Oil (BAB) | 5 | ml | Aloe Vera Leaf Gel | 275 | g |
| | | | (AVLG) | | |
| Bees Wax (BW) | 40 | g | Arginine (ARG) | 5 | g |
| Benzoin (BZN) | 4 | ml | Distilled water | 500 | ml |
| Benzyl Alcohol (BA) | 50 | ml | Ferulic Acid (FRA) | 10 | g |
| Cabreuva Oil | 5 | ml | Glutamine (GLU) | 5 | g |
| Calendula Oil (CDA) | 5 | ml | Glycine (GLY) | 5 | g |
| Camellia Oil (CMA) | 10 | ml | Histidine (HST) | 5 | g |
| Capaiba Oil (CPO) | 10 | ml | Hydroxyproline (HDP) | 5 | g |
| Caprylic, Capric | 10 | ml | Isoleucine (ISL) | 5 | g |
| Triglyceride (CCT) | | | | | |
| Carvacrol (CVC) | 5 | ml | Lecithin (LCT) | 25 | g |
| Caryophyllene (CAP) | 10 | ml | Leucine (LUC) | 5 | g |
| Cedarwood essential Oil | 2.5 | ml | Linoleic Acid (LOA) | 5 | ml |
| Cetyl Alcohol (CTA) | 40 | g | Lysine (LYS) | 5 | g |
| Chamomile German Oil | 10 | ml | Methyl Sulfonyl | 25 | g |
| (CGB) | | | Methane (MSM) | | |
| Ghana Shea Butter | 275 | g | Niacinamide (NCM) | 2.5 | g |
| (GSB) | | | | | |
| Glycerol (GLY) | 10 | ml | Oleic Acid (OLA) | 5 | ml |
| Kaempferol | 5 | g | Praline (PRL) | 5 | g |
| Kanuka Oil (KNO) | 2.5 | ml | Protocatechuic Acid | 10 | g |
| | | | (PCT) | | |
| Limonene Oil (LMN) | 40 | ml | Sericin | 50 | g |
| Litsea Cubeba Oil (LCO) | 5 | ml | Sodium Polyacrylate | 20 | g |
| Niaouli Oil | 5 | ml | Vitamin A (VTA) | 2.5 | g |
| Black Cumin Seed Oil | 50 | ml | Vitamin B3 (VTB3) or | 12.5 | g |
| (BCS) | | | Niacin | | |
| Panthenol (PNT) | 10 | ml | Vitamin C (VTC) | 2.5 | g |
| Peppermint | 5 | ml | Vitamin E (VTE) | 2.5 | g |
| Sea Buckthorn Oil (SBO) | 5 | ml | | | |
| Thyme Red, White | 5 | ml | | | |
| Winter Savory | 5 | ml | | | |

### Example 22

The methods involved in producing these compositions are generally similar but have different components for each composition. There are hydrophobic components and hydrophilic components that must be initially mixed separately under different temperature controls and then combined together properly. As many of these components are not liquid at room temperature, heating to phase change so they all become liquids is the first requirement. The first temperature level for the first mixture is set at 40° C. to 50° C., preferably 45° C., with a 300 to 500 rotations per minute (RPM), preferably 400 RPM, propeller mixing speed. All of the components that are a liquid or become a liquid at that temperature are mixed together in one of two vessels: a hydrophobic vessel and a hydrophilic vessel. The remaining unmixed components require higher temperatures to become liquid. Thus, the temperatures of the mixtures are increased to 60° C. to 80° C., preferably 60° C., and the speed of rotation is increased to 700 to 900 RPM, preferably 800 RPM. The remaining hydrophobic group components are added to its vessel and the hydrophilic group components are added to its vessel. After sufficient time of 1 to 8 hours, preferably more than 8 hours, for mixing and completion of reaction in each of the two vessels, the mixed hydrophilic group of components is then poured into the hydrophobic group vessel, maintaining the 700 to 900 RPM, preferably 800 RPM and the 60° C. to 80° C., preferably 60° C., temperature for another 10-40 minutes, preferably 20 minutes, of reaction time. At this point, the mixture is slowly cooled to room temperature over several hours of 2 to 10 hours while reducing the mixing speed to 500-700 RPM, preferably 600 RPM, continuing to insulate the mixing container. This lets the mixture cool to ambient room temperature of 25° C. to 37° C., preferably 25° C., over a slow period time while continuously being thoroughly mixed. When cooled to room temperature, the propeller is stopped, and this final mixing vessel is removed to a surface top. This final composition will remain semisolid until it is returned to partial liquid at 37° C. by design to melt onto the skin when applied. Filling individual pumps (15 ml to 50 ml) is the final step prior to storage and then for delivery of this composition to the purchaser on a regular basis.

### Example 23

Primary human adult dermal fibroblast (hDFa) cells were obtained commercially from Lifeline Cell Technologies. The hDFa cells were cultured under sterile conditions using FibroLife fibroblast media, also from Lifeline Cell Technologies and containing the recommended Supplements and Growth Factors. Cells were propagated under standard conditions in T75 and/or T150 culture flasks.

Dermal myofibroblast (hDMFa) cell were obtained through differentiation of hDFa cells by plating the hDFa cells at low density (approx. 500 cell per cm²) and growing until confluent. To detect myofibroblast cell viability, the cell proliferation assay MTS reagent sulfonyl-tetrazolium salt was obtained from Abcam plc, Product No. ab223881, CAS No. 138169-43-4. The assay was performed as described by the manufacturer.

hDMFa myofibroblast cells were cultured in a 96-well plate containing 200 μL of media and grown to approximately 80-90% confluence. The growth serum was removed and replaced with 200 μL of fresh serum-free media containing varying concentrations of inhibitor. The cells were then incubated for an additional 8-12 hours in the inhibitor media. Post incubation cell viability was measured by adding MTS reagent to each well (10-20 μL), including treated and untreated negative control wells. Absorbance of the resulting formazan (formed in viable cells only) was measured using a 96-well plate reader after 3-6 hours of incubation at 37° C. Inhibition data for each well was normalized as a percentage of the negative control and are reported in the tables below. Results ranged from low (+) to high (+++++) inhibitory activity.

TABLE 14
Compound Myofibroblast Cytotoxicity Activity

| Cmpd | | | | | |
|---|---|---|---|---|---|
| No. | Chemical Name | CAS No. | MW | Molecular Formula | Activity |
| 1 | (−)-Epicatechin gallate | 1257-08-5 | 442.37 | C22H18O10 | ++ |
| 2 | Z-Ligustilide | 4431-01-0 | 190.24 | C12H14O2 | ++ |
| 3 | α-Asarone | 2883-98-9 | 208.25 | C12H16O3 | ++ |
| 4 | α-Estradiol | 57-91-0 | 272.38 | C18H24O2 | ++++ |
| 5 | α-Phellandrene | 99-83-2 | 136.23 | C10H16 | ++ |
| 6 | α-Terpineol, 95% | 98-55-5 | 154.25 | C10H18O | + |
| 7 | β-Carotene | 7235-40-7 | 536.87 | C40H56 | +++ |
| 8 | β-Caryophyllene | 87-44-5 | 204.35 | C15H24 | +++ |
| 9 | γ-Linolenic Acid | 506-26-3 | 278.4 | C18H30O2 | +++ |
| 10 | (+)- Corydaline | 518-69-4 | 369.44 | C22H27NO4 | ++ |
| 11 | (+)-Camphor | 464-49-3 | 152.23 | C10H16O | ++ |
| 12 | (+)-Ketoconazole | 142128-59-4 | 531.43 | C26H28Cl2N4O4 | +++ |
| 13 | (±)-Sulfinpyrazone | 57-96-5 | 404.48 | C23H20N2O3S | ++++ |
| 14 | (±)-Atenolol | 29122-68-7 | 266.3 | C14H22N2O3 | +++ |
| 15 | (±)-Baclofen | 1134-47-0 | 213.7 | C10H12ClNO2 | +++ |
| 16 | (±)-Menthol | 89-78-1 | 156.15 | C10H20O | ++ |
| 17 | (±)-Palmitoylcarnitine | 6865-14-1 | 436.07 | C23H46ClNO4 | ++ |
| | chloride | | | | |
| 18 | (±)-α-Pinene | 80-56-8 | 136.23 | C10H16 | ++ |
| 19 | (1R)-(+)-α-Pinene | 7785-70-8 | 136.23 | C10H16 | + |
| 20 | (1S)-(−)-Verbenone | 1196-01-6 | 150.22 | C10H14O | ++ |
| 21 | (1S)-(−)-α-Pinene | 7785-26-4 | 136.23 | C10H16 | ++ |
| 22 | (1S)-(−)-β-Pinene | 18172-67-3 | 136.23 | C10H16 | ++ |
| 23 | (2S,3R,4S,5R)-2- | 5505-63-5 | 215.63 | C6H14ClNO5 | ++ |
| | Amino-3,4,5,6- | | | | |
| | tetrahydroxyhexanal | | | | |
| | hydrochloride | | | | |
| 24 | (R)-(−)-Carvone | 6485-40-1 | 150.22 | C10H14O | ++ |
| 25 | (R)-(+)-1,2- | 1200-22-2 | 206.33 | C8H14O2S2 | ++ |
| | Dithiolane-3- | | | | |
| | pentanoic acid | | | | |
| 26 | (R)-(+)-Limonene | 5989-27-5 | 136.23 | C10H16 | + |
| 27 | (R)-Crizotinib | 877399-52-5 | 450.34 | C21H22Cl2FN5O | ++++ |
| 28 | (S)-(+)-Carvone | 2244-16-8 | 150.22 | C10H14O | ++ |
| 29 | (S)-Crizotinib | 877399-52-5; | 450.34 | C21H22Cl2FN5O | ++++ |
| | | 1374356-45-2 | | | |
| 30 | (S)-Flurbiprofen | 51543-39-6 | 244.3 | C15H13FO2 | ++++ |
| 31 | (S)-Ketoprofen | 22161-81-5 | 254.3 | C16H14O3 | ++++ |
| 32 | (S)-Naproxen | 22204-53-1 | 230.3 | C14H14O3 | ++ |
| 33 | 1,2-Dioleoyl-3- | 132172-61-3 | 698.54 | C42H80ClNO4 | +++ |
| | trimethylammoniumpropane | | | | |
| | (chloride) | | | | |
| 34 | 1,2-Octanediol | 1117-86-8 | 146.23 | C8H18O2 | ++ |
| 35 | 1,2-propanediol | 57-55-6 | 76.09 | C3H8O2 | ++ |
| 36 | 1,4-Benzoquinone | 106-51-4 | 108.09 | C6H4O2 | ++ |
| 37 | 1,5-Dihydro-4H- | 184789-03-5 | 136.11 | C5H4N4O | + |
| | pyrazolo[3,4-d] | | | | |
| | Pyrimidin-4-one | | | | |
| 38 | 1,8-Octanediol | 629-41-4 | 146.23 | C8H18O2 | + |
| 39 | 10-Undecenoic acid | 112-38-9 | 184.28 | C11H20O2 | ++ |
| 40 | 17-AAG (KOS953) | 75747-14-7 | 585.7 | C31H43N3O8 | +++ |
| 41 | 1-Hexadecanol | 36653-82-4 | 242.44 | C16H34O | ++ |
| 42 | 1-Octanol | 111-87-5 | 130.23 | C8H18O | ++ |
| 43 | 1-Tetradecanol | 112-72-1 | 214.39 | C14H30O | + |
| 44 | 2-Aminoethanethiol | 60-23-1 | 77.15 | C2H7NS | ++ |
| 45 | 2-APB | 524-95-8 | 225.1 | C14H16BNO | ++++ |
| 46 | 2-Deoxy-D-ribose | 533-67-5 | 134.13 | C5H10O4 + adduct | ++++ |
| 47 | 2-Deoxyuridine | 951-78-0 | 228.2 | C9H12N2O5 | ++ |
| 48 | 2-Hydroxybenzyl | 90-01-7 | 124.14 | C7H8O2 | ++ |
| | alcohol | | | | |
| 49 | 2-Methoxyestradiol | 362-07-2 | 302.41 | C19H26O3 | ++ |
| | (2-MeOE2) | | | | |
| 50 | 3-5- | 99-10-5 | 154.12 | C7H6O4 | ++ |
| | Dihydroxybenzoic | | | | |
| | Acid | | | | |
| 51 | 3-acetyl-11-keto-β- | 67416-61-9 | 512.7 | C32H48O5 | +++++ |
| | Boswellic Acid | | | | |
| 52 | 3-Hydroxy-2-methyl- | 118-71-8 | 126.11 | C6H6O3 | ++ |
| | 4H-pyran-4-one | | | | |
| 53 | 3-isopropyl-5- | 3228-03-3 | 150.22 | C10H14O | + |
| | methylphenol | | | | |
| 54 | 3-n-Butylphathlide | 6066-49-5 | 190.24 | C12H14O2 | ++ |
| 55 | 3-Phenoxybenzoic | 3739-38-6 | 214.22 | C13H10O3 | ++ |
| | acid | | | | |
| 56 | 4-(Aminomethyl) | 56-91-7 | 151.16 | C8H9NO2 | ++ |
| | benzoic acid | | | | |
| 57 | 4-Aminobenzoic acid | 150-13-0 | 137.14 | C7H7NO2 | ++++ |
| 58 | 4-Aminophenol | 123-30-8 | 109.13 | C6H7NO | +++ |
| 59 | 4-Aminosalicylic acid | 65-49-6 | 153.14 | C7H7NO3 | ++ |
| 60 | 4-Biphenylacetic acid | 5728-52-9 | 212.24 | C14H12O2 | ++ |
| 61 | 4-Hydroxybenzoic | 99-96-7 | 138.12 | C7H6O3 | ++++ |
| | acid | | | | |
| 62 | 4-Methylbenzylidene | 36861-47-9 | 254.4 | C18H22O | +++ |
| | camphor | | | | |
| 63 | 4- | 90-33-5 | 176.17 | C10H8O3 | ++++ |
| | Methylumbelliferone | | | | |
| | (4-MU) | | | | |
| 64 | 5-Azacytidine | 320-67-2 | 244.2 | C8H12N4O5 | +++ |
| 65 | 5-hydroxy-L- | 4350-09-8 | 220.22 | C11H12N2O3 | ++ |
| | Tryptophan | | | | |
| 66 | 5-hydroxymethyl-2- | 67-47-0 | 126.11 | C6H6O3 | ++ |
| | furaldehyde | | | | |
| 67 | 5-Methoxypsoralen | 484-20-8 | 216.19 | C12H8O4 | ++ |
| 68 | 5-Methoxytryptamine | 608-07-1 | 190.24 | C11H14N2O | ++++ |
| 69 | 6,7- | 305-01-1 | 178.14 | C9H6O4 | ++ |
| | Dihydroxycoumarin | | | | |
| 70 | 6-Aminonicotinamide | 329-89-5 | 137.1 | C6H7N3O | +++ |
| 71 | 6H05 | 1469338-01-9 | 590.14 | C22H31ClF3N3O4S3 | ++ |
| 72 | 6-Hydroxydopamine | 636-00-0 | 250.09 | C8H11NO3•HBr | ++++ |
| | hydrobromide | | | | |
| 73 | 6-Methoxy-2- | 2471-70-7 | 202.21 | C12H10O3 | ++ |
| | naphthoic acid | | | | |
| 74 | 8-Hydroxyquinoline | 148-24-3 | 145.16 | C9H7NO | ++ |
| 75 | Abiraterone acetate | 154229-18-2 | 391.55 | C26H33NO2 | +++ |
| 76 | ABT-199 | 1257044-40-8 | 868.44 | C45H50ClN7O7S | ++++ |
| 77 | ABT-263 | 923564-51-6 | 974.61 | C47H55ClF3N5O6S3 | +++++ |
| | (Navitoclax) | | | | |
| 78 | Acetamide | 60-35-5 | 59.07 | C2H5NO | ++ |
| 79 | Acetanilide | 103-84-4 | 135.16 | C8H9NO | ++++ |
| 80 | Acetate gossypol | 12542-36-8 | 578.61 | C32H34O10 | +++++ |
| 81 | Acetohydroxamic | 546-88-3 | 75.07 | C2H5NO2 | ++++ |
| | Acid | | | | |
| 82 | Acetophenone | 98-86-2 | 120.15 | C8H8O | ++ |
| 83 | Acetylcholine iodide | 2260-50-6 | 273.11 | C7H16INO2 | ++ |
| 84 | Acitretin | 55079-83-9 | 326.43 | C21H26O3 | +++ |
| 85 | ACP-196 | 1420477-60-6 | 465.51 | C26H23N7O2 | ++++ |
| 86 | Actinomycin D | 50-76-0 | 1255.43 | C62H86N12O16 | ++ |
| 87 | Adapalene | 106685-40-9 | 412.52 | C28H28O3 | ++ |
| 88 | Adapalene sodium | 911110-93-5 | 434.5 | C28H27NaO3 | +++ |
| | salt | | | | |
| 89 | Adefovir Dipivoxil | 142340-99-6 | 501.47 | C20H32N5O8P | ++++ |
| 90 | Adenosine 5′- | 18422-05-4 | 365.24 | C10H16N5O8P | ++ |
| | monophosphate | | | | |
| | monohydrate | | | | |
| 91 | Adenosine 5- | 34369-07-8 | 507.18 | C10H16N5O13P3 | +++ |
| | triphosphate | | | | |
| 92 | Adenosine-5′- | 58-64-0 | 427.2 | C10H15N5O10P2 | ++ |
| | diphosphate | | | | |
| 93 | Afatinib (BIBW2992) | 439081-18-2 | 485.94 | C24H25ClFN5O3 | ++++ |
| 94 | Afatinib dimaleate | 850140-73-7 | 718.08 | C32H33ClFN5O11 | ++++ |
| 95 | Afuresertib | 1047644-62-1 | 427.32 | C18H17Cl2FN4OS | ++++ |
| | (GSK2110183) | | | | |
| 96 | AG-120 | 1448347-49-6 | 582.96 | C28H22ClF3N6O3 | ++++ |
| 97 | Alantolactone | 546-43-0 | 232.31 | C15H20O2 | ++++ |
| 98 | Alfacalcidol | 41294-56-8 | 400.65 | C27H44O2 | +++ |
| 99 | Allopregnanolone | 516-54-1 | 318.49 | C21H34O2 | ++++ |
| 100 | Alogliptin Benzoate | 850649-62-6 | 461.51 | C25H27N5O4 | +++ |
| 101 | Alosetron | 122852-69-1 | 330.81 | C17H19ClN4O | ++++ |
| | Hydrochloride | | | | |
| 102 | Altrenogest | 850-52-2 | 310.43 | C21H26O2 | +++ |
| 103 | Amifampridine | 54-96-6 | 109.13 | C5H7N3 | ++ |
| 104 | Amikacin | 37517-28-5 | 585.6 | C22H43N5O13 | ++++ |
| 105 | Amiloride HCl | 17440-83-4 | 302.12 | C6H8ClN7O•HCl•2H2O | ++ |
| | dihydrate | | | | |
| 106 | Aminophylline | 317-34-0 | 420.43 | C16H24N10O4 | +++ |
| 107 | Amiodarone HCl | 19774-82-4 | 681.77 | C25H30ClI2NO3 | +++ |
| 108 | Amitraz | 33089-61-1 | 293.41 | C19H23N3 | +++++ |
| 109 | Amlexanox | 68302-57-8 | 298.29 | C16H14N2O4 | +++ |
| 110 | Amlodipine | 88150-42-9 | 408.88 | C20H25ClN2O5 | +++ |
| 111 | Amlodipine Besylate | 111470-99-6 | 567.05 | C20H25ClN2O5•C6H6O3S | ++++ |
| 112 | Amlodipine maleate | 88150-47-4 | 524.95 | C24H29ClN2O9 | +++++ |
| 113 | Amorolfine HCl | 78613-38-4 | 353.97 | C21H36ClNO | +++ |
| 114 | Amoxapine | 14028-44-5 | 313.78 | C17H16ClN3O | ++ |
| 115 | Amsacrine | 51264-14-3 | 393.46 | C21H19N3O3S | +++ |
| 116 | Amsacrine | 54301-15-4 | 429.92 | C21H20ClN3O3S | +++ |
| | hydrochloride | | | | |
| 117 | Amuvatinib (MP-470, | 850879-09-3 | 447.51 | C23H21N5O3S | ++ |
| | HPK 56) | | | | |
| 118 | Amygdalin | 29883-15-6 | 457.42 | C20H27NO11 | ++ |
| 119 | AN-2690 | 174671-46-6 | 151.93 | C7H6BFO2 | +++ |
| 120 | Andrographolide | 5508-58-7 | 350.44 | C20H30O5 | ++ |
| 121 | Androsterone | 53-41-8 | 290.44 | C19H30O2 | ++ |
| 122 | Anethole | 104-46-1 | 148.2 | C10H12O | ++ |
| 123 | Anethole trithione | 532-11-6 | 240.36 | C10H8OS3 | ++ |
| 124 | Anidulafungin | 166663-25-8 | 1140.24 | C58H73N7O17 | +++++ |
| 125 | Anisodamine | 55869-99-3 | 305.37 | C17H23NO4 | ++++ |
| 126 | Aprepitant | 170729-80-3 | 534.43 | C23H21F7N4O3 | ++++ |
| 127 | Apronal | 528-92-7 | 184.24 | C9H16N2O2 | ++ |
| 128 | Arbidol HCl | 131707-23-8 | 513.88 | C22H25BrN2O3S•HCl | +++ |
| 129 | Aripiprazole | 129722-12-9 | 448.39 | C23H27Cl2N3O2 | +++ |
| 130 | Aristolochic Acid A | 313-67-7 | 341.27 | C17H11NO7 | ++ |
| 131 | ARN-509 | 956104-40-8 | 477.43 | C21H15F4N5O2S | ++++ |
| 132 | Artesunate | 88495-63-0 | 384.42 | C19H28O8 | +++ |
| 133 | Ascomycin(FK 520) | 104987-12-4 | 792.01 | C43H69NO12 | +++ |
| 134 | Asenapine | 85650-56-2 | 401.84 | C17H16ClNO•C4H4O4 | ++ |
| 135 | Asiatic acid | 464-92-6 | 488.7 | C30H48O5 | +++++ |
| 136 | Astemizole | 68844-77-9 | 458.57 | C28H31FN4O | ++++ |
| 137 | Asunaprevir (BMS- | 630420-16-5 | 748.29 | C35H46ClN5O9S | ++++ |
| | 650032) | | | | |
| 138 | Atazanavir | 198904-31-3 | 704.9 | C38H52N6O7 | ++ |
| 139 | Atipamezole | 104075-48-1 | 248.75 | C14H16N2•HCl | ++ |
| | hydrochloride | | | | |
| 140 | Atorvastatin Calcium | 134523-03-8 | 1155.34 | 2(C33H34FN2O5)•Ca | ++++ |
| 141 | ATP | 56-65-5 | 507.18 | C10H16N5O13P3 | ++ |
| 142 | Atracurium Besylate | 64228-81-5 | 1243.48 | C53H72N2O12•2(C6H5O3S) | +++ |
| 143 | Atropine | 51-55-8 | 289.37 | C17H23NO3 | |
| 144 | Atropine sulfate | 55-48-1 | 676.82 | C34H48N2O10S | ++ |
| 145 | Auranofin | 34031-32-8 | 678.48 | C20H34AuO9PS | +++++ |
| 146 | AZD-9291 | 1421373-65-0 | 499.61 | C28H33N7O2 | +++++ |
| 147 | Azelate | 123-99-9 | 188.22 | C9H16O4 | ++ |
| | (nonanedioate) | | | | |
| 148 | Azelnidipine | 123524-52-7 | 582.65 | C33H34N4O6 | +++++ |
| 149 | Azilsartan Medoxomil | 863031-21-4 | 568.53 | C30H24N4O8 | ++ |
| 150 | Bacitracin Zinc | 1405-89-6 | 1486.06 | C66H101N17O16SZn | ++ |
| 151 | BAF312 (Siponimod) | 1230487-00-9 | 516.6 | C29H35F3N2O3 | +++ |
| 152 | Bafetinib (INNO-406) | 887650-05-7 | 576.62 | C30H31F3N8O | +++ |
| 153 | Baicalein | 491-67-8 | 270.24 | C15H10O5 | ++ |
| 154 | Balofloxacin | 151060-21-8 | 425.45 | C20H28FN3O6 | ++ |
| | Dihydrate | | | | |
| 155 | Baloxavir marboxil | 1985606-14-1 | 571.55 | C27H23F2N3O7S | +++++ |
| 156 | Bardoxolone methyl | 218600-53-4 | 505.69 | C32H43NO4 | +++++ |
| 157 | Bay 11-7085 | 196309-76-9 | 249.33 | C13H15NO2S | +++++ |
| 158 | Bay 11-7821(BAY | 19542-67-7 | 207.25 | C10H9NO2S | +++++ |
| | 11-7082) | | | | |
| 159 | Bazedoxifene acetate | 198481-33-3 | 530.65 | C32H38N2O5 | ++++ |
| 160 | Benactyzine | 57-37-4 | 363.88 | C20H26ClNO3 | ++ |
| | hydrochloride | | | | |
| 161 | Benidipine HCl | 91599-74-5 | 542.02 | C28H31N3O6•HCl | ++ |
| 162 | Benserazide HCl | 14919-77-8 | 293.7 | C10H15N3O5•HCl | ++ |
| 163 | Benzamidine HCl | 1670-14-0 | 156.61 | C7H9ClN2 | +++ |
| 164 | Benzbromarone | 3562-84-3 | 424.08 | C17H12Br2O3 | ++++ |
| 165 | Benzethonium | 121-54-0 | 448.08 | C27H42NO2•Cl | +++++ |
| | Chloride | | | | |
| 166 | Benzocaine | 94-09-7 | 165.19 | C9H11NO2 | ++ |
| 167 | Benzoic Acid | 65-85-0 | 122.12 | C7H6O2 or C6H5COOH | ++ |
| 168 | Benzophenone | 119-61-9 | 182.22 | C13H10O | ++ |
| 169 | Benzyl alcohol | 100-51-6 | 108.14 | C7H8O or C6H5CH2OH | ++++ |
| 170 | Benzylpenicillin | 113-98-4 | 372.48 | C16H17KN2O4S | ++ |
| | potassium | | | | |
| 171 | Berberine | 2086-83-1 | 336.36 | C20H18NO4 | ++ |
| 172 | Berberine | 633-65-8 | 371.81 | C20H18ClNO4 | ++ |
| | hydrochloride | | | | |
| 173 | Berberine Sulfate | 633-66-9 | 433.43 | C20H19NO8S | ++ |
| 174 | beta-Alanine | 107-95-9 | 89.09 | C3H7NO2 | ++++ |
| 175 | Betahistine mesylate | 54856-23-4 | 328.41 | C10H20N2O6S2 | ++ |
| 176 | Betamethasone | 2152-44-5 | 476.58 | C27H37FO6 | ++ |
| | Valerate | | | | |
| 177 | Betamipron | 3440-28-6 | 193.2 | C10H11NO3 | +++++ |
| 178 | Bexarotene | 153559-49-0 | 348 | C24H28O2 | ++++ |
| 179 | BHT | 128-37-0 | 220.35 | C15H24O | ++ |
| 180 | BI6727 (Volasertib) | 755038-65-4 | 618.83 | C34H50N8O3 | ++++ |
| 181 | BIBR-1048 | 211915-06-9 | 627.73 | C34H41N7O5 | +++ |
| 182 | Bifonazole | 60628-96-8 | 310.39 | C22H18N2 | +++ |
| 183 | BIRB 796 | 285983-48-4 | 527.66 | C31H37N5O3 | ++ |
| | (Doramapimod) | | | | |
| 184 | Birinapant (TL32711) | 1260251-31-7 | 806.94 | C42H56F2N8O6 | ++ |
| 185 | Bisacodyl | 603-50-9 | 361.39 | C22H19NO4 | ++ |
| 186 | Bisphenol A | 80-05-7 | 228.29 | C15H16O2 | ++ |
| 187 | Blonanserin | 132810-10-7 | 367.5 | C23H30FN3 | +++ |
| 188 | Boceprevir | 394730-60-0 | 519.68 | C27H45N5O5 | ++ |
| 189 | Bosentan | 147536-97-8 | 551.63 | C27H29N5O6S | ++ |
| 190 | Bosutinib (SKI-606) | 380843-75-4 | 530.45 | C26H29Cl2N5O3 | +++ |
| 191 | Brexpiprazole | 913611-97-9 | 433.57 | C25H27N3O2S | +++ |
| 192 | Bromhexine HCl | 611-75-6 | 412.59 | C14H20Br2N2•HCl | +++ |
| 193 | Bronopol | 52-51-7 | 199.99 | C3H6BrNO4 | +++++ |
| 194 | Broxyquinoline | 521-74-4 | 302.95 | C9H5Br2NO | +++ |
| 195 | Butamben | 94-25-7 | 193.24 | C11H15NO2 | ++ |
| 196 | Butane diacid | 110-15-6 | 118.09 | C4H6O4 | ++ |
| 197 | Butenafine HCl | 101827-46-7 | 353.93 | C23H28ClN | +++ |
| 198 | Butoconazole nitrate | 64872-77-1 | 474.79 | C19H17Cl3N2S•HNO3 | +++ |
| 199 | Cabozantinib malate | 1140909-48-3 | 635.59 | C32H30FN3O10 | ++ |
| | (XL184) | | | | |
| 200 | Caffeic acid | 331-39-5 | 180.16 | C9H8O4 | + |
| | | 501-16-6 | | | |
| 201 | Caffeic Acid | 104594-70-9 | 284.31 | C17H16O4 | ++ |
| | Phenethyl Ester | | | | |
| 202 | Calcifediol | 19356-17-3 | 400.64 | C27H44O2 | +++ |
| 203 | Calcipotriol | 112965-21-6 | 412.60 | C27H40O3 | +++ |
| 204 | Calcitriol | 32222-06-3 | 416.64 | C27H44O3 | +++ |
| 205 | Calcium dobesilate | 20123-80-2 | 418.41 | C12H10CaO10S2 | ++ |
| 206 | Calcium Gluceptate | 17140-60-2 | 490.42 | C14H26CaO16 | ++++ |
| 207 | Calenduladiol | 10070-48-1 | 442.7 | C30H50O2 | + |
| 208 | Canagliflozin | 842133-18-0 | 444.52 | C24H25FO5S | +++++ |
| 209 | Canagliflozin | 928672-86-0 | 453.52 | C24H26FO5•5S | +++++ |
| | (hemihydrate) | | | | |
| 210 | Candesartan | 139481-59-7 | 440.45 | C24H20N6O3 | ++ |
| 211 | Candesartan Cilexetil | 145040-37-5 | 610.66 | C33H34N6O6 | +++ |
| 212 | Caprate (10:0) | 334-48-5 | 172.26 | C10H20O2 | ++ |
| 213 | Capsaicin | 404-86-4 | 293.40 | C17H27NO3 | +++ |
| 214 | Carbamoylcholine | 51-83-2 | 182.65 | C6H15ClN2O2 | ++ |
| | chloride | | | | |
| 215 | Carbenoxolone | 7421-40-1 | 614.72 | C34H48Na2O7 | ++++ |
| | disodium | | | | |
| 216 | Carboplatin | 41575-94-4 | 371.26 | C6H12N2O4Pt | +++ |
| 217 | Carfilzomib (PR-171) | 868540-17-4 | 719.91 | C40H57N5O7 | ++ |
| 218 | Carmustine | 154-93-8 | 214.05 | C5H9Cl2N3O2 | ++ |
| 219 | Carteolol HCl | 51781-21-6 | 328.83 | C16H25ClN2O3 | ++ |
| 220 | Carvacrol | 499-75-2 | 150.22 | C10H14O | ++ |
| 221 | Carvedilol | 72956-09-3 | 406.47 | C24H26N2O4 | +++ |
| 222 | Caryophyllene Oxide | 1139-30-6 | 220.35 | C15H24O | ++ |
| 223 | Caspofungin | 162808-62-0 | 1092.33 | C53H89N9O15 | +++ |
| 224 | Caspofungin Acetate | 179463-17-3 | 1213.42 | C56H96N10O19 | ++++ |
| 225 | CB-5083 | 1542705-92-9 | 413.47 | C24H23N5O2 | ++ |
| 226 | Cediranib (AZD217) | 288383-20-0 | 450.51 | C25H27FN4O3 | ++++ |
| 227 | Cefadroxil (hydrate) | 66592-87-8 | 381.4 | C16H17N3O5S•H2O | ++ |
| 228 | Cefazolin (sodium | 27164-46-1 | 476.5 | C14H13N8O4S3•Na | +++ |
| | salt) | | | | |
| 229 | Cefditoren Pivoxil | 117467-28-4 | 620.72 | C25H28N6O7S3 | ++ |
| 230 | Cefetamet pivoxil | 111696-23-2 | 548.03 | C20H26ClN5O7S2 | +++++ |
| | hydrochloride | | | | |
| 231 | Cefixime | 79350-37-1 | 453.4 | C16H15N5O7S2 | ++++ |
| 232 | Cefonicid (sodium | 61270-78-8 | 586.5 | C18H16N6O8S3•2Na | ++++ |
| | salt) | | | | |
| 233 | Cefotaxime (sodium | 64485-93-4 | 477.5 | C16H16N5O7S2•Na | +++ |
| | salt) | | | | |
| 234 | Cefoxitin (sodium | 33564-30-6 | 449.4 | C16H16N3O7S2•Na | +++++ |
| | salt) | | | | |
| 235 | Cefprozil | 92665-29-7 | 389.43 | C18H19N3O5S | ++ |
| 236 | Cefsulodin (sodium | 52152-93-9 | 554.5 | C22H19N4O8S2•Na | ++++ |
| | salt) | | | | |
| 237 | Ceftiofur sodium | 104010-37-9 | 545.54 | C19H16N5NaO7S3 | ++ |
| 238 | Cefuroxime axetil | 64544-07-6 | 510.47 | C20H22N4O10S | ++ |
| 239 | Cellobiose | 528-50-7 | 342.3 | C12H22O11 | +++ |
| 240 | CEP-18770 | 847499-27-8 | 413.3 | C21H28BN3O5 | ++ |
| 241 | Cepharanthine | 481-49-2 | 606.71 | C37H38N2O6 | ++ |
| 242 | Cetrimonium | 57-09-0 | 364.45 | C19H42N•Br | +++++ |
| | Bromide (CTAB) | | | | |
| 243 | Cetylpyridinium | 123-03-5 | 339.99 | C21H38N•Cl | +++++ |
| | Chloride | | | | |
| 244 | Chelerythrine | 34316-15-9 | 383.83 | C21H18ClNO4 | +++++ |
| | Chloride | | | | |
| 245 | Chenodeoxycholic | 474-25-9 | 392.57 | C24H40O4 | +++ |
| | Acid | | | | |
| 246 | Chlorcyclizine | 14362-31-3 | 337.3 | C18H21ClN2•HCl | ++ |
| | (hydrochloride) | | | | |
| 247 | Chlorhexidine | 56-95-1 | 625.55 | C26H38Cl2N10O4 | +++++ |
| | diacetate | | | | |
| 248 | Chlorhexidine | 18472-51-0 | 897.76 | C34H54Cl2N10O14 | +++ |
| | digluconate | | | | |
| 249 | Chlorhexidine HCl | 3697-42-5 | 578.37 | C22H30Cl2N10•2HCl | +++++ |
| 250 | Chloropyramine | 6170-42-9 | 326.26 | C16H21Cl2N3 | ++ |
| | hydrochloride | | | | |
| 251 | Chloroxine | 773-76-2 | 214.05 | C9H5Cl2NO | ++++ |
| 252 | Chloroxylenol | 88-04-0 | 156.61 | C8H9ClO | ++ |
| 253 | Chlorpromazine HCl | 69-09-0 | 355.33 | C17H19ClN2S•HCl | +++ |
| 254 | Chlorprothixene | 113-59-7 | 315.87 | C18H18ClNS | +++ |
| 255 | Chlorprothixene | 6469-93-8 | 352.3 | C18H18ClNS•HCl | +++ |
| | (hydrochloride) | | | | |
| 256 | Chlortetracycline HCl | 64-72-2 | 515.34 | C22H23ClN2O8•HCl | ++ |
| 257 | Choline bitartrate | 87-67-2 | 253.25 | C9H19NO7 | +++ |
| 258 | Ciclopirox | 29342-05-0 | 207.27 | C12H17NO2 | ++++ |
| 259 | Ciclopirox | 41621-49-2 | 268.35 | C12H17NO2•C2H7NO | +++ |
| | ethanolamine | | | | |
| 260 | Cilastatin | 82009-34-5 | 358.45 | C16H26N2O5S | ++++ |
| 261 | Cilengitide | 188968-51-6 | 588.66 | C27H40N8O7 | ++++ |
| 262 | Cilnidipine | 132203-70-4 | 492.52 | C27H28N2O7 | ++ |
| 263 | Cinacalcet | 226256-56-0 | 357.41 | C22H22F3N | +++ |
| 264 | Cinacalcet HCl | 364782-34-3 | 393.87 | C22H23ClF3N | +++ |
| 265 | Cinchophen | 132-60-5 | 249.26 | C16H11NO2 | ++ |
| 266 | Cinnamic acid | 140-10-3 | 148.17 | C9H8O2 | ++ |
| 267 | Cinnarizine | 298-57-7 | 368.51 | C26H28N2 | ++ |
| 268 | Ciprofloxacin | 93107-08-5 | 367.8 | C17H18FN3O3•HCl | ++ |
| | (hydrochloride) | | | | |
| 269 | Ciprofloxacin | 86393-32-0 | 385.82 | C17H21ClFN3O4 | ++ |
| | hydrochloride hydrate | | | | |
| 270 | Citalopram | 59729-32-7 | 405.31 | C20H21FN2O•HBr | ++++ |
| | hydrobromide | | | | |
| 271 | Citicoline (sodium | 33818-15-4 | 510.31 | C14H25N4NaO11P2 | +++ |
| | salt) | | | | |
| 272 | Citiolone | 1195-16-0 | 159.21 | C6H9NO2S | ++ |
| 273 | Citric acid | 77-92-9 | 192.12 | C6H8O7 | ++++ |
| 274 | Clarithromycin | 81103-11-9 | 747.95 | C38H69NO13 | +++ |
| 275 | Clemastine Fumarate | 14976-57-9 | 459.96 | C21H26ClNO•C4H4O4 | +++ |
| 276 | Clemizole | 442-52-4 | 325.84 | C19H20ClN3 | +++ |
| 277 | Clemizole | 1163-36-6 | 362.3 | C19H21Cl2N3 | ++ |
| | hydrochloride | | | | |
| 278 | Clenbuterol | 21898-19-1 | 313.7 | C12H18Cl2N2O•HCl | ++ |
| | (hydrochloride) | | | | |
| 279 | Clevidipine Butyrate | 167221-71-8 | 456.32 | C21H23Cl2NO6 | ++ |
| 280 | Clindamycin | 58207-19-5 | 479.46 | C18H36Cl2N2O6S | ++ |
| | alcoholate | | | | |
| 281 | Clioquinol | 130-26-7 | 305.5 | C9H5ClINO | ++ |
| 282 | Clodronate (sodium | 88416-50-6 | 286.9 | CH7C12NaO7P2 | +++ |
| | salt hydrate) | | | | |
| 283 | Clofazimine | 2030-63-9 | 473.4 | C27H22Cl2N4 | ++ |
| 284 | Clomiphene citrate | 50-41-9 | 598.08 | C26H28ClNO•C6H8O7 | +++ |
| 285 | Clomipramine HCl | 17321-77-6 | 351.31 | C19H23ClN2•HCl | ++ |
| 286 | Clorgyline | 17780-75-5 | 308.6 | C13H15Cl2NO•HCl | ++ |
| | (hydrochloride) | | | | |
| 287 | Closantel | 57808-65-8 | 663.07 | C22H14Cl2I2N2O2 | +++ |
| 288 | Closantel Sodium | 61438-64-0 | 685.06 | C22 H13 Cl2I2N2O2•Na | +++ |
| 289 | Clotrimazole | 23593-75-1 | 344.84 | C22H17ClN2 | +++ |
| 290 | Cobimetinib | 934660-93-2 | 531.31 | C21H21F3IN3O2 | ++++ |
| 291 | Colistin Sulfate | 1264-72-8 | 2801.27 | 2(C52H98N16O13)•5(H2SO4) | ++ |
| 292 | Costunolide | 553-21-9 | 232.32 | C15H20O2 | +++ |
| 293 | CP21R7 | 125314-13-8 | 317.3 | C19H15N3O2 | ++++ |
| 294 | CP-945598 HCl | 686347-12-6 | 546.88 | C25H25Cl2N7O•HCl | +++ |
| 295 | Creatine monohydrate | 6020-87-7 | 149.15 | C4H11N3O3 | ++ |
| 296 | Crenolanib (CP- | 670220-88-9 | 443.54 | C26H29N5O2 | +++++ |
| | 868596) | | | | |
| 297 | Crizotinib | 1415560-69-8 | 486.8 | C21H23Cl3FN5O | ++++ |
| | hydrochloride | | | | |
| 298 | Crotamiton | 483-63-6 | 203.28 | C13H17NO | ++ |
| 299 | Crystal Violet | 548-62-9 | 407.98 | C25H30ClN3 | +++ |
| 300 | Curcumin | 458-37-7 | 368.38 | C21H20O6 | +++ |
| 301 | Cyclandelate | 456-59-7 | 276.37 | C17H24O3 | +++ |
| 302 | Cyclo (−RGDfK) | 161552-03-0 | 603.7 | C27H41N9O7 | ++ |
| 303 | Cyclo(RGDyK) | 250612-42-1 | 847.72 | C31H43F6N9O12 | ++ |
| 304 | Cytosine | 71-30-7 | 111.1 | C4H5N3O | ++ |
| 305 | D-(−)-Fructose | 57-48-7 | 180.16 | C6H12O6 | ++ |
| 306 | D-(−)-Isoascorbic | 89-65-6 | 176.12 | C6H8O6 | + |
| | Acid | | | | |
| 307 | D-(−)-Salicin | 138-52-3 | 286.28 | C13H18O7 | ++ |
| 308 | D-(+)-Raffinose | 17629-30-0 | 594.51 | C18H42O21 | ++ |
| | (hydrate) | | | | |
| 309 | Dabigatran etexilate | 872728-81-9 | 723.84 | C35H45N7O8S | +++ |
| | mesylate | | | | |
| 310 | Dabrafenib | 1195765-45-7 | 519.56 | C23H20F3N5O2S2 | ++ |
| | (GSK2118436) | | | | |
| 311 | Dabrafenib Mesylate | 1195768-06-9 | 615.67 | C24H24F3N5O5S3 | ++ |
| | (GSK-2118436) | | | | |
| 312 | Daclatasvir (BMS- | 1009119-64-5 | 738.88 | C40H50N8O6 | ++++ |
| | 790052) | | | | |
| 313 | Dacomitinib | 1110813-31-4 | 469.94 | C24H25ClFN5O2 | +++ |
| | (PF299804, PF299) | | | | |
| 314 | Daidzein | 486-66-8 | 254.24 | C15H10O4 | ++ |
| 315 | Daidzin | 552-66-9 | 416.37 | C21H20O9 | ++ |
| 316 | D-Alanine | 338-69-2 | 89.09 | C3H7NO2 | +++++ |
| 317 | Danazol | 17230-88-5 | 337.5 | C22H27NO2 | ++++ |
| 318 | Danthron | 117-10-2 | 240.21 | C14H8O4 | ++ |
| 319 | Dantrolene (sodium | 24868-20-0 | 399.29 | C14H16N4NaO8.5 | ++ |
| | hemiheptahydrate) | | | | |
| 320 | Dantrolene, sodium | 14663-23-1 | 336.23 | C14H9N4NaO5 | ++++ |
| | salt | | | | |
| 321 | Dapagliflozin ((2S)- | 960404-48-2 | 502.98 | C24H35ClO9 | ++ |
| | 1,2-propanediol, | | | | |
| | hydrate) | | | | |
| 322 | Dapoxetine HCl | 129938-20-1 | 341.87 | C21H23NO•HCl | ++ |
| 323 | Dapsone | 80-08-0 | 248.3 | C12H12N2O2S | ++ |
| 324 | DAPT (GSI-IX) | 208255-80-5 | 432.46 | C23H26F2N2O4 | ++ |
| 325 | Darunavir | 206361-99-1 | 547.66 | C27H37N3O7S | ++ |
| 326 | Dasatinib (BMS- | 302962-49-8 | 488.01 | C22H26ClN7O2S | +++ |
| | 354825) | | | | |
| 327 | Dasatinib | 854001-07-3 | 524.47 | C22H27Cl2N7O2S | +++ |
| | hydrochloride | | | | |
| 328 | Dasatinib | 863127-77-9 | 506.02 | C22H28ClN7O3S | +++ |
| | Monohydrate | | | | |
| 329 | Daunorubicin HCl | 23541-50-6 | 563.98 | C27H30ClNO10 | +++ |
| 330 | D-Biotin | 58-85-5 | 244.31 | C10H16N2O3S | + |
| 331 | Decanoic acid | 334-48-5 | 172.26 | C10H20O2 | + |
| 332 | Decitabine | 2353-33-5 | 228.08 | C8H12N4O4 | +++ |
| | (NSC127716, 5AZA- | | | | |
| | CdR) | | | | |
| 333 | Defactinib | 1073154-85-4 | 510.49 | C20H21F3N8O3S | ++++ |
| 334 | Dehydroandrographolide | 134418-28-3 | 332.42 | C20H28O4 | ++ |
| 335 | Dehydrocholic acid | 81-23-2 | 402.52 | C24H34O5 | ++ |
| 336 | Dehydrocostus | 477-43-0 | 230.3 | C15H18O2 | +++++ |
| | Lactone | | | | |
| 337 | Dehydroepiandrosterone | 853-23-6 | 330.46 | C21H30O3 | ++ |
| | acetate | | | | |
| 338 | delta-Gluconolactone | 90-80-2 | 178.14 | C6H10O6 | ++ |
| 339 | Deoxycholic acid | 302-95-4 | 414.55 | C24H39NaO4 | +++++ |
| | sodium salt | | | | |
| 340 | Dequalinium Chloride | 522-51-0 | 527.57 | C30H40N4.2Cl | ++ |
| 341 | D-erythro-Sphingosine | 123-78-4 | 299.5 | C18H37NO2 | ++ |
| | (synthetic) | | | | |
| 342 | Desipramine | 58-28-6 | 302.84 | C18H22N2•HCl | ++++ |
| | hydrochloride | | | | |
| 343 | Desloratadine | 100643-71-8 | 310.82 | C19H19ClN2 | ++ |
| 344 | Desogestrel | 54024-22-5 | 310.47 | C22H30O | +++ |
| 345 | Dexlansoprazole | 138530-94-6 | 369.36 | C16H14F3N3O2S | ++ |
| 346 | D-Glutamine | 5959-95-5 | 146.14 | C5H10N2O3 | ++ |
| 347 | Diacerein | 13739-02-1 | 368.29 | C19H12O8 | ++++ |
| 348 | Diatrizoic acid | 117-96-4 | 613.91 | C11H9I3N2O4 | ++ |
| 349 | Diaveridine | 5355-16-8 | 260.29 | C13H16N4O2 | ++ |
| 350 | Dibenzothiophene | 132-65-0 | 184.26 | C12H8S | ++ |
| 351 | Dibucaine | 85-79-0 | 343.46 | C20H29N3O2 | ++ |
| 352 | Dibutyryl-cAMP, | 16980-89-5 | 491.37 | C18H23N5NaO8P | ++ |
| | sodium salt | | | | |
| 353 | Dichlorophen | 97-23-4 | 269.12 | C13H10Cl2O2 | +++++ |
| 354 | Diclazuril | 101831-37-2 | 407.64 | C17H9Cl3N4O2 | +++ |
| 355 | Diclofenac | 15307-86-5 | 296.15 | C14H11Cl2NO2 | ++ |
| 356 | Diclofenac Sodium | 15307-79-6 | 318.13 | C14H10Cl2NNaO2 | ++ |
| 357 | Dicyclomine HCl | 67-92-5 | 345.95 | C19H35NO2•HCl | ++ |
| 358 | Diethylstilbestrol | 56-53-1 | 268.35 | C18H20O2 | +++ |
| 359 | Diflunisal | 22494-42-4 | 250.2 | C13H8F2O3 | ++ |
| 360 | Digoxin | 20830-75-5 | 780.94 | C41H64O14 | ++++ |
| 361 | Dihydroartemisinin | 71939-50-9 | 284.35 | C15H24O5 | ++ |
| 362 | Diiodohydroxyquinoline | 83-73-8 | 396.95 | C9H5I2NO | ++ |
| 363 | Dimenhydrinate | 523-87-5 | 469.96 | C24H28ClN5O3 | ++ |
| 364 | Dioscin | 19057-60-4 | 869.05 | C45H72O16 | +++++ |
| 365 | Diphenidol HCl | 3254-89-5 | 345.91 | C21H28ClNO | ++++ |
| 366 | Disopyramide | 3737-09-5 | 339.47 | C21H29N3O | ++ |
| 367 | Dithranol | 1143-38-0 | 226.23 | C14H10O3 | ++ |
| 368 | DL-Dithiothreitol | 3483-12-3 | 154.25 | C4H10O2S2 | ++++ |
| 369 | DL-Methionine | 59-51-8 | 149.21 | C5H11NO2S | ++ |
| 370 | DL-Panthenol | 16485-10-2 | 205.25 | C9H19NO4 | +++ |
| 371 | DL-α- | 96020-91-6 | 236.6 | C6H12F2N2O2•HCl | ++ |
| | Difluoromethylornithine | | | [H2O] | |
| | (hydrochloride | | | | |
| | hydrate) | | | | |
| 372 | DL-α-Lipoic Acid | 1077-28-7 | 206.33 | C8H14O2S2 | ++++ |
| 373 | DL-α-Tocopherol | 10191-41-0 | 430.71 | C29H50O2 | ++ |
| 374 | Docetaxel Trihydrate | 148408-66-6 | 861.93 | C43H59NO17 | ++ |
| 375 | Docosahexaenoic | 6217-54-5 | 328.49 | C22H32O2 | +++ |
| | Acid | | | | |
| 376 | Dolasetron | 115956-12-2 | 324.37 | C19H20N2O3 | ++ |
| 377 | Domiphen Bromide | 538-71-6 | 414.46 | C22H40NO•Br | +++++ |
| 378 | Domperidone | 57808-66-9 | 425.91 | C22H24ClN5O2 | ++ |
| 379 | Doramectin | 117704-25-3 | 899.11 | C50H74O14 | +++++ |
| 380 | Dorsomorphin 2HCl | 1219168-18-9 | 472.41 | C24H25N5O•2HCl | ++ |
| 381 | Dovitinib (TKI258) | 915769-50-5 | 500.52 | C24H29FN6O5 | +++ |
| | Lactate | | | | |
| 382 | Dovitinib (TKI-258, | 405169-16-6 | 392.43 | C21H21FN6O | ++ |
| | CHIR-258) | | | | |
| 383 | Dovitinib Dilactic | 852433-84-2 | 572.59 | C21H21FN6O•2C3H6O3 | +++ |
| | acid | | | | |
| 384 | Doxazosin Mesylate | 77883-43-3 | 547.58 | C23H25N5O5•CH4O3S | +++ |
| 385 | Doxepin | 1229-29-4 | 315.8 | C19H21NO•HCl | ++ |
| | (hydrochloride) | | | | |
| 386 | Doxercalciferol | 54573-75-0 | 412.66 | C28H44O2 | +++ |
| 387 | Doxifluridine | 3094-09-5 | 246.19 | C9H11FN2O5 | ++++ |
| 388 | Doxycycline HCl | 10592-13-9 | 480.9 | C22H24N2O8•HCl | ++ |
| 389 | D-Pantethine | 16816-67-4 | 554.72 | C22H42N4O8S2 | ++ |
| 390 | D-Panthenol | 81-13-0 | 205.25 | C9H19NO4 | ++ |
| 391 | D-Pantothenate | 867-81-2 | 241.22 | C9H16NNaO5 | ++ |
| | sodium | | | | |
| 392 | D-Phenylalanine | 673-06-3 | 165.19 | C9H11NO2 | ++ |
| 393 | D-Ribose | 50-69-1 | 150.13 | C5H10O5 | ++ |
| 394 | Dronedarone | 141626-36-0 | 556.77 | C31H44N2O5S | +++ |
| 395 | Dronedarone HCl | 141625-93-6 | 593.22 | C31H45ClN2O5S | +++ |
| 396 | D-Tartaric acid | 147-71-7 | 150.09 | C4H6O6 | ++ |
| 397 | Duloxetine HCl | 136434-34-9 | 333.88 | C18H19NOS•HCl | +++++ |
| 398 | Dyclonine HCl | 536-43-6 | 325.87 | C18H27NO2•HCl | +++ |
| 399 | Dyphylline | 479-18-5 | 254.24 | C10H14N4O4 | ++ |
| 400 | E 64d | 88321-09-9 | 342.43 | C17H30N2O5 | ++ |
| 401 | Ebastine | 90729-43-4 | 469.66 | C32H39NO2 | +++ |
| 402 | Econazole | 27220-47-9 | 381.68 | C18H15Cl3N2O | +++++ |
| 403 | Econazole nitrate | 24169-02-6 | 444.7 | C18H15Cl3N2O•HNO3 | +++ |
| 404 | edetate calcium | 62-33-9 | 374.27 | C10H12CaN2Na2O8 | ++ |
| | disodium | | | | |
| 405 | Edoxaban | 480449-70-5 | 548.06 | C24H30ClN7O4S | ++ |
| 406 | Edrophonium | 116-38-1 | 201.7 | C10H16NO•Cl | +++ |
| | (chloride) | | | | |
| 407 | Efavirenz | 154598-52-4 | 315.68 | C14H9ClF3NO2 | +++ |
| 408 | Eicosapentaenoic | 10417-94-4 | 302.45 | C20H30O2 | +++ |
| | Acid | | | | |
| 409 | Elacridar | 143664-11-3 | 563.64 | C34H33N3O5 | ++ |
| 410 | Eltrombopag | 496775-61-2 | 442.47 | C25H22N4O4 | ++ |
| 411 | Eltrombopag Olamine | 496775-62-3 | 564.63 | C29H36N6O6 | ++ |
| 412 | Elvitegravir (GS- | 697761-98-1 | 447.9 | C23H23ClFNO5 | ++++ |
| | 9137) | | | | |
| 413 | EMD-1214063 | 1100598-32-0 | 492.57 | C29H28N6O2 | ++++ |
| 414 | Emeramide | 351994-94-0 | 284.4 | C12H16N2O2S2 | ++ |
| 415 | Emodin (6-Methyl- | 518-82-1 | 270.24 | C15H10O5 | ++ |
| | 1,3,8- | | | | |
| | trihydroxyanthraquinone) | | | | |
| 416 | Enoxacin | 84294-96-2 | 347.34 | C15H20FN4O4•5 | ++ |
| | Sesquihydrate | | | | |
| 417 | Enoxolone | 471-53-4 | 470.68 | C30H46O4 | +++ |
| 418 | Entecavir Hydrate | 209216-23-9 | 295.29 | C12H15N5O3•H2O | +++ |
| 419 | Entrectinib | 1108743-60-7 | 560.64 | C31H34F2N6O2 | +++ |
| 420 | Enzastaurin | 170364-57-5 | 515.61 | C32H29N5O2 | ++++ |
| | (LY317615) | | | | |
| 421 | Epiandrosterone | 481-29-8 | 290.44 | C19H30O2 | ++ |
| 422 | Epothilone B | 152044-54-7 | 507.68 | C27H41NO6S | ++ |
| | (EPO906, Patupilone) | | | | |
| 423 | Eprodisate disodium | 36589-58-9 | 248.19 | C3H6Na2O6S2 | ++ |
| 424 | Eptifibatide Acetate | 148031-34-9 | 892.01 | C37H53N11O11S2 | ++ |
| 425 | Erlotinib | 183321-74-6 | 393.44 | C22H23N3O4 | ++ |
| 426 | Esculin | 531-75-9 | 340.28 | C15H16O9•xH2O | ++ |
| 427 | Esomeprazole | 161973-10-0 | 713.12 | C34H36MgN6O6S2 | ++ |
| | Magnesium | | | | |
| 428 | Esomeprazole | 217087-09-7 | 767.17 | C34H42MgN6O9S2 | ++ |
| | Magnesium trihydrate | | | | |
| 429 | Estradiol Benzoate | 50-50-0 | 376.49 | C25H28O3 | +++ |
| 430 | Estradiol Cypionate | 313-06-4 | 396.56 | C26H36O3 | +++ |
| 431 | Estradiol valerate | 979-32-8 | 356.5 | C23H32O3 | +++ |
| 432 | Estropipate | 7280-37-7 | 436.56 | C18H22O5S•C4H10N2 | +++ |
| 433 | Ethacridine lactate | 6402-23-9 | 361.39 | C15H15N3O•C3H6O3•H2O | ++++ |
| | monohydrate | | | | |
| 434 | Ethacrynic Acid | 58-54-8 | 303.14 | C13H12Cl2O4 | +++ |
| 435 | Ethidium bromide | 1239-45-8 | 394.31 | C21H20BrN3 | ++ |
| 436 | Ethosuximide | 77-67-8 | 141.17 | C7H11NO2 | ++ |
| 437 | Ethyl Cinnamate | 103-36-6 | 176.21 | C11H12O2 | ++ |
| 438 | Ethyl gallate | 831-61-8 | 198.17 | C9H10O5 | ++ |
| 439 | Ethynodiol diacetate | 297-76-7 | 384.51 | C24H32O4 | ++ |
| 440 | Etidronate disodium | 7414-83-7 | 249.99 | C2H6Na2O7P2 | ++++ |
| 441 | Etofenamate | 30544-47-9 | 369.34 | C18H18F3NO4 | +++ |
| 442 | Etretinate | 54350-48-0 | 354.48 | C23H30O3 | +++ |
| 443 | Eupatilin | 22368-21-4 | 344.32 | C18H16O7 | ++ |
| 444 | Evacetrapib | 1186486-62-3 | 638.65 | C31H36F6N6O2 | +++ |
| | (LY2484595) | | | | |
| 445 | Evans Blue | 314-13-6 | 960.82 | C34H24N6Na4O14S4 | ++ |
| | tetrasodium salt | | | | |
| 446 | Everolimus | 159351-69-6 | 958.22 | C53H83NO14 | +++++ |
| | (RAD001) | | | | |
| 447 | Famciclovir | 104227-87-4 | 321.33 | C14H19N5O4 | +++ |
| 448 | Felbamate | 25451-15-4 | 238.24 | C11H14N2O4 | +++ |
| 449 | Felodipine | 72509-76-3 | 384.25 | C18H19Cl2NO4 | +++ |
| 450 | Fendiline | 13636-18-5 | 351.9 | C23H25N•HCl | +++ |
| | (hydrochloride) | | | | |
| 451 | Fenretinide | 65646-68-6 | 391.55 | C26H33NO2 | +++ |
| 452 | Fenticonazole Nitrate | 73151-29-8 | 518.41 | C24H20Cl2N2OS•HNO3 | ++++ |
| 453 | Ferulic Acid | 537-98-4 | 194.18 | C10H10O4 | ++ |
| | | 1135-24-6 | | | |
| 454 | Fidaxomicin | 873857-62-6 | 1058.04 | C52H74Cl2O18 | +++++ |
| 455 | Fingolimod (FTY720) | 162359-56-0 | 343.94 | C19H34ClNO2 | ++++ |
| 456 | Flavin adenine | 84366-81-4 | 829.51 | C27H31N9Na2O15P2 | ++++ |
| | dinucleotide disodium | | | | |
| 457 | Flavone | 525-82-6 | 222.24 | C15H10O2 | ++ |
| 458 | Flopropione | 2295-58-1 | 182.17 | C9H10O4 | ++ |
| 459 | Floxuridine | 50-91-9 | 246.19 | C9H11FN2O5 | +++ |
| 460 | Flunarizine 2HCl | 30484-77-6 | 477.42 | C26H26F2N2•2HCl | ++ |
| 461 | Flunisolide | 3385-03-3 | 434.5 | C24H31FO6 | ++ |
| 462 | Fluorescein | 2321-07-5 | 332.31 | C20H12O5 | ++ |
| 463 | Fluorometholone | 426-13-1 | 376.46 | C22H29FO4 | ++ |
| 464 | Fluoxetine HCl | 56296-78-7 | 345.79 | C17H18F3NO•HCl | +++ |
| 465 | Fluphenazine | 146-56-5 | 510.44 | C22H28Cl2F3N3OS | +++++ |
| | dihydrochloride | | | | |
| 466 | Fluvoxamine maleate | 61718-82-9 | 434.41 | C14H18F3N2O2•C4H4O4 | ++ |
| 467 | Folic acid | 59-30-3 | 441.4 | C19H19N7O6 | ++ |
| 468 | Folinic Acid (calcium | 1492-18-8 | 511.5 | C20H21CaN707 | +++ |
| | salt) | | | | |
| 469 | Foretinib | 849217-64-7 | 632.65 | C34H34F2N4O6 | +++ |
| | (GSK1363089) | | | | |
| 470 | Fosaprepitant | 265121-04-8 | 1004.83 | C37H56F7N6O16P | +++ |
| | dimeglumine salt | | | | |
| 471 | Fostamatinib (R788) | 901119-35-5 | 580.46 | C23H26FN6O9P | ++ |
| 472 | Fruquintinib(HMPL- | 1194506-26-7 | 393.39 | C21H19N3O5 | ++++ |
| | 013) | | | | |
| 473 | Fudosteine | 13189-98-5 | 179.24 | C6H13NO3S | ++++ |
| 474 | Fumalic acid | 1135-24-6 | 194.18 | C10H10O4 | ++ |
| 475 | Furazolidone | 67-45-8 | 225.16 | C8H7N3O5 | ++ |
| 476 | Furfural | 98-01-1 | 96.08 | C5H4O2 | ++ |
| 477 | Fusidic Acid (sodium | 751-94-0 | 538.7 | C31H47O6•Na | +++ |
| | salt) | | | | |
| 478 | Gallic acid | 149-91-7 | 170.12 | C7H6O5 | ++ |
| 479 | GDC-0941 | 957054-30-7 | 513.64 | C23H27N7O3S2 | ++++ |
| 480 | Gefitinib (ZD1839) | 184475-35-2 | 446.90 | C22H24ClFN4O3 | +++ |
| 481 | Gefitinib | 184475-55-6 | 483.36 | C22H25Cl2FN4O3 | +++ |
| | hydrochloride | | | | |
| 482 | Germacrone | 6902-91-6 | 218.17 | C15H22O | +++++ |
| 483 | GHRP-2 | 158861-67-7 | 817.97 | C45H55N9O6 | ++ |
| 484 | Ginkgolide C | 15291-76-6 | 440.4 | C20H24O11 | ++ |
| 485 | Gliquidone | 33342-05-1 | 527.63 | C27H33N3O6S | ++ |
| 486 | Glycerol | 56-81-5 | 92.09 | C3H8O3 | ++ |
| 487 | Glycine (GLY) | 56-40-6 | 75.07 | C2H5NO2 | ++++ |
| 488 | Glycocholic Acid | 475-31-0 | 465.62 | C26H43NO6 | ++++ |
| 489 | Glycylglycine | 556-50-3 | 132.12 | C4H8N2O3 | ++ |
| 490 | Gossypol | 303-45-7 | 518.55 | C30H30O8 | +++++ |
| 491 | GS-9973 | 1229208-44-9 | 411.46 | C23H21N7O | ++++ |
| 492 | GSK1349572 sodiuM | 1051375-19-9 | 441.36 | C20H18F2N3NaO5 | ++++ |
| | salt | | | | |
| 493 | GSK2126458 | 1086062-66-9 | 505.5 | C25H17F2N5O3S | ++++ |
| 494 | Guaiacol | 90-05-1 | 124.14 | C7H8O2 | +++ |
| 495 | Guanethidine (sulfate) | 645-43-2 | 296.39 | C10H24N4O4S | ++++ |
| 496 | GW501516 | 317318-70-0 | 453.5 | C21H18F3NO3S2 | +++ |
| 497 | GZD824 | 1421783-64-3 | 724.77 | C29H27F3N6O•2CH4O3S | +++++ |
| 498 | Harmine | 442-51-3 | 212.25 | C13H12N2O | ++++ |
| 499 | Harpagoside | 19210-12-9 | 494.18 | C24H30O11 | ++ |
| 500 | Helicid | 80154-34-3 | 284.06 | C13H16O7 | ++ |
| 501 | Herniarin | 531-59-9 | 176.17 | C10H8O3 | ++ |
| 502 | Hesperidin | 520-26-3 | 610.56 | C28H34O15 | ++ |
| 503 | Hesperitin | 520-33-2 | 302.29 | C16H14O6 | ++ |
| 504 | Hexachlorophene | 70-30-4 | 406.9 | C13H6Cl6O2 | +++++ |
| 505 | Hexamethylcyclotrisiloxane | 541-05-9 | 222.46 | C6H18O3Si3 | + |
| 506 | Hexestrol | 84-16-2 | 270.37 | C18H22O2 | +++ |
| 507 | Honokiol | 35354-74-6 | 266.33 | C18H18O2 | +++++ |
| 508 | Hydroquinidine | 1435-55-8 | 326.43 | C20H26N2O2 | ++ |
| 509 | Hydroquinone | 123-31-9 | 110.11 | C6H6O2 | ++ |
| 510 | Hydroxylammonium | 5470-11-1 | 69.49 | ClH4NO | ++ |
| | chloride | | | | |
| 511 | Hydroxy-L-Proline | 618-28-0 | 131.13 | C5H9NO3 | + |
| 512 | Hyodeoxycholic acid | 83-49-8 | 392.56 | C24H40O4 | ++ |
| 513 | Ibudilast | 50847-11-5 | 230.3 | C14H18N2O | +++ |
| 514 | Icatibant Acetate | 138614-30-9 | 1364.57 | C61H93N19O15S | ++ |
| 515 | Idarubicin HCl | 57852-57-0 | 533.95 | C26H28ClNO9 | +++++ |
| 516 | Idebenone | 58186-27-9 | 338.44 | C19H30O5 | +++ |
| 517 | Idramantone | 20098-14-0 | 166.22 | C10H14O2 | ++ |
| 518 | Ifosfamide | 3778-73-2 | 261.09 | C7H15Cl2N2O2P | +++ |
| 519 | Imatinib (STI571) | 152459-95-5 | 493.6 | C29H31N7O | ++ |
| 520 | Imatinib Mesylate | 220127-57-1 | 589.71 | C29H31N7O•CH4SO3 | +++ |
| | (STI571) | | | | |
| 521 | IMD 0354 | 978-62-1 | 383.67 | C15H8ClF6NO2 | ++++ |
| 522 | Imidazole | 288-32-4 | 68.08 | C3H4N2 | ++++ |
| 523 | Imipramine | 113-52-0 | 316.9 | C19H24N2•HCl | +++ |
| | (hydrochloride) | | | | |
| 524 | INCB-024360 | 1204669-58-8 | 438.23 | C11H13BrFN7O4S | ++ |
| 525 | Indacaterol | 312753-06-3 | 392.49 | C24H28N2O3 | +++ |
| 526 | Indacaterol Maleate | 753498-25-8 | 508.56 | C24H28N2O3•C4H4O4 | +++ |
| 527 | Indapamide | 26807-65-8 | 365.83 | C16H16ClN3O3S | +++ |
| 528 | Indigo carmine | 860-22-0 | 466.35 | C16H8N2Na2O8S2 | ++ |
| 529 | Indobufen | 63610-08-2 | 295.33 | C18H17NO3 | ++ |
| 530 | Ingenol | 30220-46-3 | 348.43 | C20H28O5 | +++ |
| 531 | Iniparib (BSI-201) | 160003-66-7 | 292.03 | C7H5IN2O3 | +++++ |
| 532 | Iohexol | 66108-95-0 | 821.14 | C19H26I3N3O9 | ++++ |
| 533 | Iopromide | 73334-07-3 | 791.11 | C18H24I3N3O8 | +++ |
| 534 | Isavuconazole | 241479-67-4 | 437.47 | C22H17F2N5OS | ++ |
| 535 | Isoconazole nitrate | 24168-96-5 | 479.14 | C18H14Cl4N2O•HNO3 | +++ |
| 536 | Isoeugenol | 97-54-1 | 164.2 | C10H12O2 | ++ |
| 537 | Isonicotinic acid | 55-22-1 | 123.11 | C6H5NO2 | ++++ |
| 538 | Isoprinosine | 36703-88-5 | 1115.2 | C10H12N4O5•3C9H9NO3•3C5H13NO | ++ |
| 539 | Isoproterenol sulfate | 299-95-6 | 556.62 | C22H40N2O12S | ++ |
| | dihydrate | | | | |
| 540 | Isosorbide 5- | 16051-77-7 | 191.14 | C6H9NO6 | ++ |
| | mononitrate | | | | |
| 541 | Isosorbide Dinitrate | 87-33-2 | 236.14 | C6H8N2O8 | ++++ |
| 542 | Isoxsuprine | 579-56-6 | 337.8 | C18H23NO3•HCl | ++ |
| | (hydrochloride) | | | | |
| 543 | Ispinesib (SB- | 336113-53-2 | 517.06 | C30H33ClN4O2 | +++++ |
| | 715992) | | | | |
| 544 | Isradipine (Dynacirc) | 75695-93-1 | 371.39 | C19H21N3O5 | ++ |
| 545 | Ivacaftor (VX-770) | 873054-44-5 | 392.49 | C24H28N2O3 | +++++ |
| 546 | Ivermectin | 70288-86-7 | 875.09 | C48H74O14 | ++++ |
| 547 | K02288 | 1431985-92-0 | 352.38 | C20H20N2O4 | ++ |
| 548 | Kasugamycin | 19408-46-9 | 415.8 | C14H25N3O9•HCl | +++ |
| | (hydrochloride) | | | | |
| 549 | Kobe0065 | 436133-68-5 | 449.79 | C15H11ClF3N5O4S | +++ |
| 550 | L-(−)-Fucose | 2438-80-4 | 164.16 | C6H12O5 | ++ |
| 551 | L(+)-Ornithine | 3184-13-2 | 168.62 | C5H13ClN2O2 | +++++ |
| | hydrochloride | | | | |
| 552 | L-165041 | 79558-09-1 | 402.44 | C22H26O7 | ++ |
| 553 | Labetalol HCl | 32780-64-6 | 364.87 | C19H25ClN2O3 | ++ |
| 554 | Lacidipine | 103890-78-4 | 455.54 | C26H33NO6 | +++ |
| 555 | lactate | 79-33-4 | 90.08 | C3H6O3 | ++ |
| 556 | L-Alanine | 56-41-7 | 89.09 | C3H7NO2 | +++ |
| 557 | Lapatinib | 231277-92-2 | 581.06 | C29H26ClFN4O4S | +++ |
| 558 | Lapatinib Ditosylate | 388082-77-7 | 925.46 | C29H26ClFN4O4S2C7H8O3S | +++ |
| 559 | Laquinimod (ABR- | 248281-84-7 | 356.8 | C19H17ClN2O3 | ++++ |
| | 215062) | | | | |
| 560 | L-Arabinose | 5328-37-0 | 150.13 | C5H10O5 | ++++ |
| 561 | L-Arginine | 74-79-3 | 174.2 | C6H14N4O2 | ++ |
| 562 | L-Ascorbic Acid | 50-81-7 | 176.12 | C6H8O6 or HC6H7O6 | + |
| | (Vitamin C) | | | | |
| 563 | Latanoprost | 130209-82-4 | 432.59 | C26H40O5 | ++++ |
| 564 | Lauric Acid | 143-07-7 | 200.32 | C12H24O2 | ++ |
| 565 | Laurocapram | 59227-89-3 | 281.48 | C18H35NO | +++++ |
| 566 | Lawsone | 83-72-7 | 174.15 | C10H6O3 | ++ |
| 567 | L-Carnitine inner salt | 541-15-1 | 161.2 | C7H15NO3 | ++ |
| 568 | L-Carnosine | 305-84-0 | 226.23 | C9H14N4O3 | 8 |
| 569 | L-Cysteine HCl | 52-89-1 | 157.62 | C3H8ClNO2S | ++ |
| 570 | LCZ696 | 936623-90-4 | 956.99 | C48H54N6Na3O8•2.5H2O | ++ |
| 571 | LDE225 (NVP-LDE225, | 956697-53-3 | 485.5 | C26H26F3N3O3 | +++ |
| | Erismodegib) | | | | |
| 572 | LDE225 Diphosphate | 1218778-77-8 | 681.49 | C26H32F3N3O11P2 | ++ |
| 573 | LDK378 | 1032900-25-6 | 558.14 | C28H36ClN5O3S | +++++ |
| 574 | LDN193189 | 1062368-62-0 | 552.33 | C25H26Cl4N6 | +++++ |
| | Hydrochloride | | | | |
| 575 | LDN-212854 | 1432597-26-6 | 406.48 | C25H22N6 | ++++ |
| 576 | LDN-214117 | 1627503-67-6 | 419.52 | C25H29N3O3 | ++ |
| 577 | Lecithin, Soybean | 8002-43-5 | — | — | + |
| 578 | Lenalidomide (CC- | 191732-72-6 | 259.3 | C13H13N3O3 | ++ |
| | 5013) | | | | |
| 579 | Letermovir | 917389-32-3 | 572.55 | C29H28F4N4O4 | ++++ |
| 580 | Levamlodipine | 103129-82-4 | 408.88 | C20H25ClN2O5 | +++++ |
| 581 | Levosimendan | 141505-33-1 | 280.28 | C14H12N6O | ++ |
| 582 | Levothyroxine | 55-03-8 | 798.85 | C15H10I4NNaO4 | ++ |
| | sodium | | | | |
| 583 | Levulinic acid | 123-76-2 | 116.12 | C5H8O3 | ++ |
| 584 | L-Glutamic acid | 142-47-2 | 169.11 | C5H8NNaO4 | ++ |
| | monosodium salt | | | | |
| 585 | L-Glutathione | 70-18-8 | 307.32 | C10H17N3O6S | ++++ |
| | Reduced | | | | |
| 586 | L-Histidine | 71-00-1 | 155.15 | C6H9N3O2 | + |
| 587 | L-Hydroxyproline | 51-35-4 | 131.13 | C5H9NO3 | ++++ |
| 588 | Licofelone | 156897-06-2 | 379.88 | C23H22ClNO2 | +++ |
| 589 | Lidocaine | 6108-05-0 | 288.81 | C14H25ClN2O2 | ++ |
| | hydrochloride | | | | |
| | monohydrate | | | | |
| 590 | Linifanib (ABT-869) | 796967-16-3 | 375.41 | C21H18FN5O | ++ |
| 591 | Linoleic Acid | 60-33-3 | 280.45 | C18H32O2 | +++ |
| 592 | Linsitinib | 867160-71-2 | 421.51 | C26H23N5O | +++ |
| 593 | Lipex Sheasoft TR | 194043-92-0 | 150 | | + |
| 594 | L-Isoleucine (ISL) | 443-79-8 | 131.17 | C6H13NO2 | + |
| 595 | Lithium carbonate | 554-13-2 | 73.89 | Li2CO3 | ++++ |
| 596 | L-Leucine | 61-90-5 | 131.17 | C6H13NO2 | +++ |
| 597 | L-Lysine HCl | 10098-89-2 | 182.65 | C6H14N2O2 | + |
| 598 | L-Lysine | 657-27-2 | 182.65 | C6H15ClN2O2 | ++++ |
| | hydrochloride | | | | |
| 599 | L-Menthol | 2216-51-5 | 156.27 | C10H20O | ++ |
| 600 | L-Methionine | 63-68-3 | 149.21 | C5H11NO2S | ++++ |
| 601 | Loganin | 18524-94-2 | 390.38 | C17H26O10 | ++ |
| 602 | Lomerizine HCl | 101477-54-7 | 541.46 | C27H30F2N2O3•2HCl | +++ |
| 603 | Lomitapide | 182431-12-5 | 692.71 | C39H37F6N3O2 | +++ |
| 604 | Lomustine | 13010-47-4 | 233.7 | C9H16ClN3O2 | ++ |
| 605 | Lonafarnib | 193275-84-2 | 638.82 | C27H31Br2ClN4O2 | +++ |
| 606 | Loperamide HCl | 34552-83-5 | 513.5 | C29H33ClN2O2•HCl | +++ |
| 607 | Lopinavir | 192725-17-0 | 628.81 | C37H48N4O5 | ++++ |
| 608 | Loratadine | 79794-75-5 | 382.88 | C22H23ClN2O2 | +++ |
| 609 | Lornoxicam | 70374-39-9 | 371.82 | C13H10ClN3O4S2 | ++ |
| 610 | Lovastatin | 75330-75-5 | 404.54 | C24H36O5 | +++ |
| 611 | Loxoprofen Sodium | 80382-23-6 | 268.28 | C15H17NaO3 | ++ |
| 612 | L-Proline | 147-85-3 | 115.13 | C5H9NO2 | ++++ |
| 613 | L-Pyroglutamic acid | 98-79-3 | 129.11 | C5H7NO3 | ++++ |
| 614 | L-Threonine | 72-19-5 | 119.12 | C4H9NO3 | +++++ |
| 615 | L-Tryptophan | 73-22-3 | 204.23 | C11H12N2O2 | ++ |
| 616 | Lutein | 127-40-2 | 568.87 | C40H56O2 | ++++ |
| 617 | Lutidinic acid | 499-80-9 | 167.12 | C7H5NO4 | ++ |
| 618 | L-Valine | 72-18-4 | 117.15 | C5H11NO2 | ++ |
| 619 | L-Xylose | 609-06-3 | 150.13 | C5H10O5 | +++ |
| 620 | LY2228820 | 862507-23-1 | 612.74 | C24H29FN6•2CH4O3S | ++++ |
| 621 | LY2835219 | 1231930-82-7 | 602.7 | C27H32F2N8•CH4O3S | ++++ |
| 622 | LY2835219 free base | 1231929-97-7 | 506.59 | C27H32F2N8 | ++++ |
| 623 | Lynestrenol | 52-76-6 | 284.44 | C20H28O | +++++ |
| 624 | Macitentan | 441798-33-0 | 588.27 | C19H20Br2N6O4S | +++ |
| 625 | Madecassic acid | 18449-41-7 | 504.7 | C30H48O6 | +++++ |
| 626 | Mafenide Acetate | 13009-99-9 | 246.28 | C9H14N2O4S | ++++ |
| 627 | Mafenide | 138-37-4 | 222.69 | C7H11ClN2O2S | ++ |
| | hydrochloride | | | | |
| 628 | Magnolol | 528-43-8 | 266.32 | C18H18O2 | +++++ |
| 629 | Maleimide | 541-59-3 | 97.07 | C4H3NO2 | +++++ |
| 630 | Malic acid | 6915-15-7 | 134.09 | C4H6O5 | ++ |
| 631 | Malonic acid | 141-82-2 | 104.06 | C3H4O4 | ++++ |
| 632 | Malotilate | 59937-28-9 | 288.38 | C12H16O4S2 | ++ |
| 633 | Maltitol | 585-88-6 | 344.31 | C12H24O11 | ++ |
| 634 | Maltotriose | 1109-28-0 | 504.44 | C18H32O16 | +++ |
| 635 | Maprotiline HCl | 10347-81-6 | 313.86 | C20H23N•HCl | +++ |
| 636 | m-Cresol | 108-39-4 | 108.14 | C7H8O | ++ |
| 637 | Mebendazole | 31431-39-7 | 295.3 | C16H13N3O3 | ++++ |
| 638 | Mebeverine | 2753-45-9 | 466.01 | C25H36ClNO5 | ++ |
| | Hydrochloride | | | | |
| 639 | Mecamylamine | 826-39-1 | 203.75 | C11H21N•HCl | ++++ |
| | hydrochloride | | | | |
| 640 | Mechlorethamine HCl | 55-86-7 | 192.51 | C5H11Cl2N•HCl | ++ |
| 641 | Meclizine 2HCl | 1104-22-9 | 463.87 | C25H27Cl2N2•2HCl | ++ |
| 642 | Meclofenamate | 6385-02-0 | 318.13 | C14H11Cl2NO2•Na | ++ |
| | Sodium | | | | |
| 643 | Medroxyprogesterone | 71-58-9 | 386.52 | C24H34O4 | ++ |
| | acetate | | | | |
| 644 | Mefloquine | 51773-92-3 | 414.77 | C17H17ClF6N2O | ++++ |
| | hydrochloride | | | | |
| 645 | Meisoindigo | 97207-47-1 | 276.29 | C17H12N2O2 | +++ |
| 646 | Melamine | 108-78-1 | 126.12 | C3H6N6 | ++ |
| 647 | Melitracen | 10563-70-9 | 327.89 | C21H26ClN | +++++ |
| | hydrochloride | | | | |
| 648 | Meloxicam (Mobic) | 71125-38-7 | 351.4 | C14H13N3O4S2 | ++ |
| 649 | Melphalan | 148-82-3 | 305.2 | C13H18Cl2N2O2 | ++ |
| 650 | Menadiol Diacetate | 573-20-6 | 258.27 | C15H14O4 | +++++ |
| 651 | Menadione | 58-27-5 | 172.18 | C11H8O2 | +++ |
| 652 | Menaquinone 4 | 863-61-6 | 444.65 | C31H40O2 | ++ |
| 653 | Menbutone | 3562-99-0 | 258.27 | C15H14O4 | ++ |
| 654 | Meptazinol HCl | 59263-76-2 | 269.81 | C15H24ClNO | ++++ |
| 655 | Methacholine chloride | 62-51-1 | 195.69 | C8H18ClNO2 | ++ |
| 656 | Methicillin (sodium | 132-92-3 | 402.4 | C17H19N2O6S•Na | ++ |
| | salt) | | | | |
| 657 | Methionine | 348-67-4 | 149.21 | C5H11NO2S | ++ |
| 658 | Methotrexate | 7413-34-5 | 498.4 | C20H20N8Na2O5 | ++ |
| | disodium | | | | |
| 659 | Methyl salicylate | 119-36-8 | 152.15 | C8H8O3 | ++ |
| 660 | Methyl Sulfonyl | 67-71-0 | 94.14 | C2H6SO2 | + |
| | Methane (MSM) | | | | |
| 661 | Methylbenactyzine | 3166-62-9 | 422.36 | C21H28BrNO3 | ++ |
| | Bromide | | | | |
| 662 | Methylcobalamin | 13422-55-4 | 1344.38 | C63H91CoN13O14P+ | ++ |
| 663 | Methylene Blue | 61-73-4 | 319.85 | C16H18ClN3S | +++++ |
| 664 | Methylparaben | 99-76-3 | 152.15 | C8H8O3 | ++ |
| 665 | Metoprolol Tartrate | 56392-17-7 | 684.81 | 2C15H25NO3•C4H6O6 | ++ |
| 666 | Metronidazole | 13182-89-3 | 275.26 | C13H13N3O4 | ++ |
| | Benzoate | | | | |
| 667 | Metyrapone | 54-36-4 | 226.27 | C14H14N2O | ++ |
| 668 | Mevastatin | 73573-88-3 | 390.51 | C23H34O5 | +++ |
| 669 | m-Hydroxybenzoic | 99-06-9 | 138.12 | C7H6O3 | ++++ |
| | acid | | | | |
| 670 | Micafungin sodium | 208538-73-2 | 1292.26 | C56H70N9NaO23S | ++++ |
| 671 | Miconazole | 22916-47-8 | 416.13 | C18H14Cl4N2O | +++ |
| 672 | Miconazole Nitrate | 22832-87-7 | 479.14 | C18H14Cl4N2O•HNO3 | +++ |
| 673 | Midodrine | 43218-56-0 | 290.7 | C12H18N2O4•HCl | ++++ |
| | (hydrochloride) | | | | |
| 674 | Midostaurin | 120685-11-2 | 570.64 | C35H30N4O4 | ++ |
| | (PKC412) | | | | |
| 675 | Miglitol | 72432-03-2 | 207.22 | C8H17NO5 | ++++ |
| 676 | Milnacipran HCl | 101152-94-7 | 282.81 | C15H23ClN2O | ++++ |
| 677 | Miltefosine | 58066-85-6 | 407.57 | C21H46NO4P | +++++ |
| 678 | Minoxidil sulphate | 83701-22-8 | 289.31 | C9H15N5O4S | ++ |
| 679 | Mirtazapine | 85650-52-8 | 265.35 | C17H19N3 | ++++ |
| 680 | Mitomycin C | 50-07-7 | 334.33 | C15H18N4O5 | ++ |
| 681 | Mitotane (Lsodren) | 53-19-0 | 320.04 | C14H10Cl4 | +++ |
| 682 | Mitoxantrone HCl | 70476-82-3 | 517.4 | C22H29ClN4O6•HCl | +++++ |
| 683 | MK-5172 | 1350514-68-9 | 766.9 | C38H50N6O9S | +++++ |
| 684 | MLN2238 | 1072833-77-2 | 361 | C14H19BCl2N2O4 | ++ |
| 685 | MLN8237 (Alisertib) | 1028486-01-2 | 518.92 | C27H20ClFN4O4 | ++++ |
| 686 | MLN9708 | 1201902-80-8 | 517.1 | C20H23BCl2N2O9 | ++ |
| 687 | Molidustat (BAY85- | 1154028-82-6 | 314.3 | C13H14N8O2 | +++ |
| | 3934) | | | | |
| 688 | Mometasone furoate | 83919-23-7 | 521.43 | C27H30Cl2O6 | ++ |
| 689 | Montelukast Sodium | 151767-02-1 | 608.17 | C35H35ClNNaO3S | +++++ |
| 690 | Motolimod (VTX- | 926927-61-9 | 458.6 | C28H34N4O2 | ++ |
| | 2337) | | | | |
| 691 | Moxidectin | 113507-06-5 | 639.82 | C37H53NO8 | ++++ |
| 692 | myo-Inositol | 87-89-8 | 180.16 | C6H12O6 + adduct | ++++ |
| 693 | Myristic Acid | 544-63-8 | 228.37 | C14H28O2 | +++ |
| 694 | N-acetyl-D- | 7512-17-6 | 221.21 | C8H15NO6 | +++ |
| | Glucosamine | | | | |
| 695 | N-Acetyl-L-cysteine | 616-91-1 | 163.2 | C5H9NO3S | + |
| 696 | Naftifine HCl | 65473-14-5 | 323.86 | C21H21N•HCl | ++ |
| 697 | Naftopidil | 57149-07-2 | 392.49 | C24H28N2O3 | +++ |
| 698 | Napabucasin | 83280-65-3 | 240.21 | C14H8O4 | ++++ |
| 699 | Natamax B Plus | — | — | — | ++ |
| 700 | Natamax Salt | — | — | — | + |
| 701 | Nebivolol | 152520-56-4 | 441.9 | C22H26ClF2NO4 | ++++ |
| 702 | Neohesperidin | 13241-33-3 | 610.56 | C28H34O15 | ++ |
| 703 | Neratinib (HKI-272) | 698387-09-6 | 557.04 | C30H29ClN6O3 | +++ |
| 704 | N-Ethylmaleimide | 128-53-0 | 125.13 | C6H7NO2 | +++++ |
| | (NEM) | | | | |
| 705 | Niacinamide | 25334-23-1 | 122.12 | C6H6N2O | + |
| 706 | Nicardipine HCl | 54527-84-3 | 515.99 | C26H29N3O6•HCl | +++ |
| 707 | Nicergoline | 27848-84-6 | 484.39 | C24H26BrN3O3 | ++ |
| 708 | Niclosamide | 50-65-7 | 327.12 | C13H8C12N2O4 | ++ |
| 709 | Nifedipine | 21829-25-4 | 346.33 | C17H18N2O6 | +++ |
| 710 | Niflumic acid | 4394-00-7 | 282.22 | C13H9F3N2O2 | ++ |
| 711 | Nifuroxazide | 965-52-6 | 275.22 | C12H9N3O5 | ++ |
| 712 | Nilotinib | 923288-90-8 | 583.99 | C28H25ClF3N7O2 | +++ |
| | monohydrochloride | | | | |
| | monohydrate | | | | |
| 713 | Nilotinib(AMN-107) | 641571-10-0 | 529.53 | C28H22F3N7O | ++++ |
| 714 | Nilutamide | 63612-50-0 | 317.22 | C12H10F3N3O4 | ++++ |
| 715 | Nilvadipine | 75530-68-6 | 385.37 | C19H19N3O6 | +++ |
| 716 | Nintedanib (BIBF | 656247-17-5 | 539.62 | C31H33N5O4 | ++++ |
| | 1120) | | | | |
| 717 | Nisoldipine | 63675-72-9 | 388.41 | C20H24N2O6 | +++ |
| 718 | Nitazoxanide | 55981-09-4 | 307.28 | C12H9N3O5S | +++ |
| 719 | Nitrendipine | 39562-70-4 | 360.3699 | C18H20N2O6 | +++ |
| 720 | Nitrofurantoin | 67-20-9 | 238.16 | C8H6N4O5 | ++++ |
| 721 | Nitroxoline | 4008-48-4 | 190.16 | C9H6N2O3 | ++++ |
| 722 | Nordihydroguaiaretic | 500-38-9 | 302.36 | C18H22O4 | +++ |
| | acid | | | | |
| 723 | Nortriptyline | 894-71-3 | 299.8 | C19H21N•HCl | ++ |
| | (hydrochloride) | | | | |
| 724 | Novobiocin Sodium | 1476-53-5 | 634.61 | C31H35N2O11•Na | ++++ |
| 725 | NSC 3852 | 3565-26-2 | 174.16 | C9H6N2O2 | ++++ |
| 726 | Nuciferine | 475-83-2 | 295.38 | C19H21NO2 | ++ |
| 727 | Obeticholic Acid | 459789-99-2 | 420.63 | C26H44O4 | +++ |
| 728 | o-Cresol | 95-48-7 | 108.14 | C7H8O | ++ |
| 729 | Octanoic acid | 124-07-2 | 144.21 | C8H16O2 | ++ |
| 730 | Octenidine | 70775-75-6 | 623.83 | C36H64Cl2N4 | +++++ |
| | (dihydrochloride) | | | | |
| 731 | ODM-201 | 1297538-32-9 | 398.85 | C19H19ClN6O2 | ++ |
| 732 | Oleanolic acid | 508-02-1 | 456.71 | C30H48O3 | +++++ |
| 733 | Oleic Acid | 112-80-1 | 282.46 | C18H34O2 | +++ |
| 734 | Olivetol | 500-66-3 | 180.24 | C11H16O2 | ++ |
| 735 | Olmutinib | 1353550-13-6 | 486.59 | C26H26N6O2S | ++++ |
| | (HM61713, BI1482694) | | | | |
| 736 | Oltipraz | 64224-21-1 | 226.34 | C8H6N2S3 | ++ |
| 737 | Omeprazole Sodium | 95510-70-6 | 367.4 | C17H18N3NaO3S | ++ |
| 738 | Orcinol | 504-15-4 | 124.14 | C7H8O2 | ++ |
| 739 | Orphenadrine | 341-69-5 | 305.84 | C18H24ClNO | ++ |
| | Hydrochloride | | | | |
| 740 | Osthole | 484-12-8 | 244.29 | C15H16O3 | ++ |
| 741 | Otilonium Bromide | 26095-59-0 | 563.57 | C29H43BrN2O4 | +++++ |
| 742 | o-Toluic acid | 118-90-1 | 136.15 | C8H8O2 | ++ |
| 743 | Ouabain | 630-60-4 | 728.77 | C29H44O12•8H2O | ++ |
| 744 | Oxaceprol | 33996-33-7 | 173.17 | C7H11NO4 | ++ |
| 745 | Oxethazaine | 126-27-2 | 467.64 | C28H41N3O3 | +++++ |
| 746 | Oxiconazole (nitrate) | 64211-46-7 | 492.1 | C18H13Cl4N3O•HNO3 | +++ |
| 747 | Oxybutynin | 5633-20-5 | 357.49 | C22H31NO3 | ++ |
| 748 | Oxyclozanide | 2277-92-1 | 401.46 | C13H6Cl5NO3 | +++++ |
| 749 | Oxymatrine | 16837-52-8 | 264.36 | C15H24N2O2 | ++ |
| 750 | Ozanimod (RPC1063) | 1306760-87-1 | 404.46 | C23H24N4O3 | +++ |
| 751 | Pacritinib (SB1518) | 937272-79-2 | 472.58 | C28H32N4O3 | +++ |
| 752 | Paeonol | 552-41-0 | 166.18 | C9H10O3 | ++ |
| 753 | Palbociclib | 827022-33-3 | 573.66 | C24H29N7O2•C2H6O4S | +++++ |
| | (PD0332991) | | | | |
| | Isethionate | | | | |
| 754 | Palmitic Acid methyl | 112-39-0 | 270.45 | C17H34O2 | +++ |
| | ester | | | | |
| 755 | Palmitoleic Acid | 373-49-9 | 254.41 | C16H30O2 | +++ |
| 756 | Pamidronate | 57248-88-1 | 279.03 | C3H9NNa2O7P2 | +++ |
| | Disodium | | | | |
| 757 | Pargyline | 306-07-0 | 195.7 | C11H13N•HCl | ++ |
| | (hydrochloride) | | | | |
| 758 | Paroxetine HCl | 78246-49-8 | 365.83 | C19H20FNO3•HCl | +++ |
| 759 | Parthenolide | 20554-84-1 | 248.32 | C15H20O3 | +++++ |
| 760 | Pazufloxacin mesylate | 163680-77-1 | 414.4 | C17H19FN2O7S | +++ |
| 761 | PCI-32765 (Ibrutinib) | 936563-96-1 | 440.5 | C25H24N6O2 | +++ |
| 762 | p-Cresol | 106-44-5 | 108.14 | C7H8O | ++ |
| 763 | PD 0332991 | 827022-32-2 | 483.99 | C24H30ClN7O2 | ++++ |
| | (Palbociclib) HCl | | | | |
| 764 | Pelitinib (EKB-569) | 257933-82-7 | 467.92 | C24H23ClFN5O2 | +++ |
| 765 | Penfluridol | 26864-56-2 | 523.97 | C28H27ClF5NO | +++ |
| 766 | Penicillamine | 52-67-5 | 149.21 | C5H11NO2S | ++ |
| 767 | Pentadecanoic Acid | 1002-84-2 | 242.4 | C15H30O2 | ++ |
| 768 | Pentamidine | 140-64-7 | 592.68 | C19H24N4O2•2C2H6O4S | +++ |
| | isethionate | | | | |
| 769 | Pentoxifylline | 6493-05-6 | 278.31 | C13H18N4O3 | ++++ |
| 770 | Pentylenetetrazole | 54-95-5 | 138.17 | C6H10N4 | ++++ |
| 771 | Perifosine | 157716-52-4 | 461.67 | C25H52NO4P | +++++ |
| 772 | Perospirone | 129273-38-7 | 463.04 | C23H31ClN4O2S | +++++ |
| | hydrochloride | | | | |
| 773 | Perphenazine | 58-39-9 | 403.97 | C21H26ClN3OS | +++++ |
| 774 | Pexidartinib | 1029044-16-3 | 417.81 | C20H15ClF3N5 | ++ |
| | (PLX3397) | | | | |
| 775 | Phenazine | 299-11-6 | 306.34 | C14H14N2O4S | +++++ |
| | methosulfate | | | | |
| 776 | Phenoxybenzamine | 63-92-3 | 340.3 | C18H22ClNO•HCl | ++ |
| | HCl | | | | |
| 777 | Phenytoin | 57-41-0 | 252.27 | C15H12N2O2 | ++ |
| 778 | Phenytoin sodium | 630-93-3 | 274.25 | C15H11N2NaO2 | ++ |
| 779 | Phloridzin | 60-81-1 | 436.41 | C21H24O10 | ++ |
| 780 | Phosphocreatine | 922-32-7 | 255.08 | C4H8N3Na2O5P | ++++ |
| | disodium salt | | | | |
| 781 | Phthalic acid | 88-99-3 | 166.13 | C8H6O4 | ++ |
| 782 | Phytol | 150-86-7 | 296.5 | C20H40O | ++ |
| 783 | Pikamilone | 34562-97-5 | 208.21 | C10H12N2O3 | ++ |
| 784 | Pimozide | 2062-78-4 | 461.6 | C28H29F2N3O | ++++ |
| 785 | Pioglitazone HCl | 112529-15-4 | 392.9 | C19H20N2O3S•HCl | +++ |
| 786 | Pitavastatin Calcium | 147526-32-7 | 880.98 | C50H46CaF2N2O8 | +++ |
| 787 | Polydatin | 27208-80-6 | 390.39 | C20H22O8 | ++ |
| 788 | Polymyxin B (sulfate) | 1405-20-5 | 1301.6 | C56H98N16O13•H2SO4 | ++ |
| 789 | Pomalidomide (CC- | 19171-19-8 | 273.2 | C13H11N3O4 | ++ |
| | 4047) | | | | |
| 790 | Ponatinib (AP24534) | 943319-70-8 | 532.56 | C29H27F3N6O | +++ |
| 791 | Poziotinib | 1092364-38-9 | 491.34 | C23H21Cl2FN4O3 | ++ |
| 792 | Pralidoxime Iodide | 94-63-3 | 264.06 | C7H9IN2O | ++ |
| 793 | Pranlukast | 103177-37-3 | 481.5 | C27H23N5O4 | ++ |
| 794 | Prasugrel | 389574-19-0 | 409.9 | C20H21ClFNO3S | ++ |
| | hydrochloride | | | | |
| 795 | Prazosin HCl | 19237-84-4 | 419.86 | C19H21N5O4•HCl | +++ |
| 796 | Primaquine | 63-45-6 | 455.34 | C15H21N3O•2H3O4P | +++ |
| | Diphosphate | | | | |
| 797 | Prochlorperazine | 58-38-8 | 373.94 | C20H24ClN3S | +++++ |
| 798 | Proflavine | 1811-28-5 | 258.25 | C13H11N3•1/2H2O4S | +++++ |
| | Hemisulfate | | | | |
| 799 | Progesterone | 57-83-0 | 314.46 | C21H30O2 | +++ |
| 800 | Promestriene | 39219-28-8 | 328.49 | C22H32O2 | ++ |
| 801 | Promethazine HCl | 58-33-3 | 320.88 | C17H21ClN2S | ++ |
| 802 | Propofol | 2078-54-8 | 178.27 | C12H18O | ++ |
| 803 | Prostaglandin F2α | 38562-01-5 | 475.62 | C20H34O5•C4H11NO3 | ++++ |
| 804 | Protocatechuic acid | — | 154.12 | C1H604 | ++ |
| 805 | Proxyphylline | 603-00-9 | 238.2 | C10H14N4O3 | ++ |
| 806 | Prucalopride | 179474-81-8 | 367.87 | C18H26ClN3O3 | +++ |
| 807 | Purine | 120-73-0 | 120.11 | C5H4N4 | ++ |
| 808 | Puromycin | 58-58-2 | 544.43 | C22H29N7O5•2HCl | ++++ |
| | dihydrochloride | | | | |
| 809 | Pyridoxine | 65-23-6 | 169.18 | C8H11NO3 | ++ |
| 810 | Pyrocatechol | 120-80-9 | 110.11 | C6H6O2 | ++ |
| 811 | Pyrrolidinedithiocarbamate | 5108-96-3 | 164.28 | C5H12N2S2 | ++ |
| | ammonium | | | | |
| 812 | Quercitrin | 522-12-3 | 448.38 | C21H20O11 | ++ |
| 813 | Quinestrol | 152-43-2 | 364.5 | C25H32O2 | ++++ |
| 814 | Quinine | 130-95-0 | 324.42 | C20H24N2O2 | ++ |
| 815 | Quizartinib (AC220) | 950769-58-1 | 560.67 | C29H32N6O4S | ++++ |
| 816 | R788 disodium | 1025687-58-4 | 624.42 | C23H24FN6Na2O9P | ++ |
| 817 | Racecadotril | 81110-73-8 | 385.48 | C21H23NO4S | ++ |
| 818 | Radotinib(IY-5511) | 926037-48-1 | 530.5 | C27H21F3N8O | ++++ |
| 819 | Raloxifene HCl | 82640-04-8 | 510.04 | C28H28ClNO4S | ++++ |
| 820 | Rapamycin | 53123-88-9 | 914.18 | C51H79NO13 | ++++ |
| | (Sirolimus) | | | | |
| 821 | Rasagiline | 136236-51-6 | 171.24 | C12H13N | ++ |
| 822 | Regorafenib | 755037-03-7 | 482.82 | C21H15ClF4N4O3 | ++ |
| 823 | Regorafenib | 835621-07-3 | 519.28 | C21H16C12F4N4O3 | ++++ |
| | hydrochloride | | | | |
| 824 | Regorafenib | 1019206-88-2 | 500.83 | C21H17ClF4N4O4 | +++ |
| | monohydrate | | | | |
| 825 | Remdesivir (GS- | 1809249-37-3 | 602.58 | C27H35N6O8P | ++ |
| | 5734) | | | | |
| 826 | Repaglinide | 135062-02-1 | 452.59 | C27H36N2O4 | +++ |
| 827 | Resazurin sodium salt | 62758-13-8 | 251.17 | C12H6NNaO4 | ++ |
| 828 | Reserpine | 50-55-5 | 608.27 | C33H40N2O9 | ++ |
| 829 | Resveratrol | 501-36-0 | 228.24 | C14H12O3 | ++ |
| 830 | Retinyl (Vitamin A) | 79-81-2 | 524.86 | C36H60O2 | ++ |
| | Palmitate | | | | |
| 831 | RG7388 | 1229705-06-9 | 616.48 | C31H29Cl2F2N3O4 | ++++ |
| 832 | Ridaforolimus | 572924-54-0 | 990.21 | C53H84NO14P | ++++ |
| | (Deforolimus, MK- | | | | |
| | 8669) | | | | |
| 833 | Rifamycin sodium | 14897-39-3 | 719.75 | C37H46NNaO12 | ++ |
| | salt | | | | |
| 834 | Rifapentine | 61379-65-5 | 877.03 | C47H64N4O12 | +++ |
| 835 | Rilpivirine | 500287-72-9 | 366.42 | C22H18N6 | ++ |
| 836 | Rimantadine | 1501-84-4 | 215.76 | C12H22ClN | ++++ |
| | Hydrochloride | | | | |
| 837 | Ro 31-8220 Mesylate | 138489-18-6 | 553.65 | C25H23N5O2S•CH4O3S | +++ |
| 838 | Robenidine | 25875-50-7 | 370.66 | C15H14Cl3N5 | ++ |
| | hydrochloride | | | | |
| 839 | Romidepsin (FK228, | 128517-07-7 | 540.7 | C24H36N4O6S2 | ++ |
| | depsipeptide) | | | | |
| 840 | Ropivacaine Mesilate | 854056-07-8 | 370.51 | C18H30N2O4S | ++ |
| 841 | Roquinimex | 84088-42-6 | 308.33 | C18H16N2O3 | ++ |
| 842 | Rosuvastatin Calcium | 147098-20-2 | 1001.14 | C44H54F2N6O12S2 Ca | +++ |
| 843 | Rufinamide | 106308-44-5 | 238.19 | C10H8F2N4O | +++ |
| 844 | Rupatadine Fumarate | 182349-12-8 | 532.03 | C26H26ClN3•C4H4O4 | +++ |
| 845 | Rutin | 153-18-4 | 610.51 | C27H30O16 | ++ |
| 846 | Saikosaponin A | 20736-09-8 | 780.99 | C42H68O13 | +++++ |
| 847 | Salbutamol | 18559-94-9 | 239.31 | C13H21NO3 | ++ |
| 848 | Salirasib | 162520-00-5 | 358.54 | C22H30O2S | +++ |
| 849 | Salmeterol xinafoate | 94749-08-3 | 603.75 | C36H45NO7 | +++ |
| 850 | Sanguinarine | 2447-54-3 | 332.33 | C20H14NO4 | +++++ |
| 851 | Sanguinarine chloride | 5578-73-4 | 367.78 | C20H14ClNO4 | ++++ |
| 852 | Saquinavir mesylate | 149845-06-7 | 766.95 | C39H54N6O8S | ++++ |
| 853 | Saracatinib | 379231-04-6 | 542.03 | C27H32ClN5O5 | +++ |
| | (AZD0530) | | | | |
| 854 | Saxagliptin hydrate | 945667-22-1 | 333.43 | C18H27N3O3 | ++ |
| 855 | Sclareol | 515-03-7 | 308.29 | C20H36O2 | +++ |
| 856 | Scopolamine HBr | 6533-68-2 | 438.31 | C17H28BrNO7 | ++ |
| | trihydrate | | | | |
| 857 | Scopoletin | 92-61-5 | 192.17 | C10H8O4 | ++ |
| 858 | sebacate | 111-20-6 | 202.25 | C10H18O4 | ++ |
| | (decanedioate) | | | | |
| 859 | Sericin Gel | 474338-68-6 | 150 | C30H40N10O16 | + |
| 860 | Sertaconazole nitrate | 99592-39-9 | 500.78 | C20H15Cl3N2OS•HNO3 | +++++ |
| 861 | Sertraline HCl | 79559-97-0 | 342.69 | C17H18Cl3N | ++++ |
| 862 | Sildenafil mesylate | 1308285-21-3 | 570.68 | C23H34N6O7S2 | ++ |
| 863 | Simeprevir | 923604-59-5 | 749.96 | C38H47N5O7S2 | +++++ |
| 864 | Simvastatin (Zocor) | 79902-63-9 | 418.6 | C25H38O5 | +++ |
| 865 | SIS3 | 521984-48-5 | 489.99 | C28H28ClN3O3 | +++ |
| 866 | Sivelestat (ONO- | 127373-66-4 | 434.46 | C20H22N2O7S | ++ |
| | 5046) | | | | |
| 867 | SKF 525A | 62-68-0 | 390.0 | C23H31NO2•HCl | +++ |
| | (hydrochloride) | | | | |
| 868 | SLx-2119 | 911417-87-3 | 452.51 | C26H24N6O2 | ++++ |
| 869 | Sodium Aescinate | 20977-05-3 | 1131.26 | C55H86O24 | +++++ |
| 870 | Sodium benzoate | 532-32-1 | 144.1 | C7H5NaO2 | ++++ |
| 871 | Sodium dodecyl | 151-21-3 | 288.38 | C12H25NaO4S | +++++ |
| | sulfate | | | | |
| 872 | Sodium Nitroprusside | 14402-89-2 | 261.92 | C5FeN6Na2O | ++++ |
| 873 | Sodium | 1716-12-7 | 186.18 | C10H11O2•Na | +++ |
| | Phenylbutyrate | | | | |
| 874 | Sodium phosphate | 7558-79-4 | 141.96 | Na2HPO4 | ++++ |
| | dibasic | | | | |
| 875 | Sodium salicylate | 54-21-7 | 160.1 | C7H5NaO3 | ++++ |
| 876 | Sodium sulfadiazine | 547-32-0 | 272.26 | C10H9N4NaO2S | ++ |
| 877 | Sodium | 35807-85-3 | 521.69 | C26H44NNaO6S | ++ |
| | Tauroursodeoxycholate | | | | |
| | (TUDC) | | | | |
| 878 | Sofalcone | 64506-49-6 | 450.52 | C27H30O6 | ++++ |
| 879 | Sophoricoside | 152-95-4 | 432.38 | C21H20O10 | ++ |
| 880 | Sorafenib Tosylate | 475207-59-1 | 637.03 | C21H16ClF3N4O3•C7H8O3S | +++ |
| 881 | Sorbic acid | 110-44-1 | 112.13 | C6H8O2 | ++ |
| 882 | Sorbitol | 50-70-4 | 182.17 | C6H14O6 | ++++ |
| 883 | Sotalol hydrochloride | 959-24-0 | 308.82 | C12H21ClN2O3S | +++ |
| 884 | Sotrastaurin | 425637-18-9 | 438.48 | C25H22N6O2 | ++ |
| | (AEB071) | | | | |
| 885 | Spectinomycin | 22189-32-8 | 495.4 | C14H24N2O7•2HCl | +++ |
| | (hydrochloride | | | [5H2O] | |
| | hydrate) | | | | |
| 886 | Spectinomycin | 21736-83-4 | 405.27 | C14H26Cl2N2O7 | +++ |
| | dihydrochloride | | | | |
| 887 | Spermine | 71-44-3 | 202.3 | C10H26N4 | ++ |
| 888 | Staurosporine | 62996-74-1 | 466.53 | C28H26N4O3 | +++++ |
| 889 | Stearic Acid | 57-11-4 | 284.48 | C18H36O2 | +++ |
| 890 | Stearic Acid methyl | 112-61-8 | 298.5 | C19H38O2 | ++ |
| | ester | | | | |
| 891 | Succinylsulfathiazole | 116-43-8 | 355.39 | C13H13N3O5S2 | ++ |
| 892 | Sulconazole Nitrate | 61318-91-0 | 460.76 | C18H15Cl3N2S•HNO3 | ++++ |
| 893 | Sulfabenzamide | 127-71-9 | 276.31 | C13H12N2O3S | ++ |
| 894 | Sulfacetamide sodium | 6209-17-2 | 254.24 | C8H11N2NaO4S | ++ |
| | salt hydrate | | | | |
| 895 | Sulfamerazine | 127-79-7 | 264.3 | C11H12N4O2S | +++ |
| 896 | Sulfamethizole | 144-82-1 | 270.33 | C9H10N4O2S2 | ++++ |
| 897 | Sulfamethoxazole | 4563-84-2 | 275.26 | C10H10N3NaO3S | ++ |
| | sodium | | | | |
| 898 | Sulfanilamide | 63-74-1 | 172.2 | C6H8N2O2S | ++++ |
| 899 | Sulfaphenazole | 526-08-9 | 314.4 | C15H14N4O2S | +++ |
| 900 | Sulfathiazole | 72-14-0 | 255.32 | C9H9N3O2S2 | ++++ |
| 901 | Sulisobenzone | 4065-45-6 | 308.31 | C14H12O6S | ++ |
| 902 | Sultamicillin | 76497-13-7 | 594.66 | C25H30N4O9S2 | ++ |
| 903 | Sultamicillin Tosylate | 83105-70-8 | 766.86 | C32H38N4O12S3 | ++ |
| 904 | Sunitinib | 557795-19-4 | 398.47 | C22H27FN4O2 | ++++ |
| 905 | Sunitinib malate | 341031-54-7 | 532.56 | C22H27FN4O2•C4H6O5 | ++++ |
| 906 | Tacrine hydrochloride | 1684-40-8 | 234.73 | C13H14N2•HCl | ++++ |
| 907 | Tacrolimus (FK506) | 104987-11-3 | 804.02 | C44H69NO12 | +++ |
| 908 | TAK-438 | 1260141-27-2 | 461.46 | C17H16FN3O2S•C4H4O4 | +++ |
| 909 | Tamibarotene | 94497-51-5 | 351.44 | C22H25NO3 | ++++ |
| 910 | Tamoxifen | 10540-29-1 | 371.51 | C26H29NO | +++ |
| 911 | Tamoxifen Citrate | 54965-24-1 | 563.64 | C32H37NO8 | +++ |
| 912 | Tanshinone I | 568-73-0 | 276.29 | C18H12O3 | +++++ |
| 913 | Tanshinone IIA | 568-72-9 | 294.34 | C19H18O3 | +++++ |
| 914 | Tanshinone IIA--- | 69659-80-9 | 396.39 | C19H17NaO6S | ++ |
| | sulfonic sodium | | | | |
| 915 | Tasisulam | 519055-62-0 | 415.11 | C11H6BrCl2NO3S2 | ++ |
| 916 | Taurolidine | 19388-87-5 | 284.4 | C7H16N4O4S2 | +++ |
| 917 | Tauroursodeoxycholic | 14605-22-2 | 499.7 | C26H45NO6S | ++ |
| | Acid | | | | |
| 918 | Tazarotene | 118292-40-3 | 351.46 | C21H21NO2S | ++ |
| 919 | Tebipenempivoxil | 161715-24-8 | 497.63 | C22H31N3O6S2 | ++ |
| 920 | Tedizolid HCl | 856866-72-3 | 406.80 | C17H16ClFN6O3 | ++++ |
| 921 | Teicoplanin | 61036-62-2 | 1709.39 | C77H77N9O31Cl2 | +++ |
| 922 | Telmisattan | 144701-48-4 | 514.62 | C33H30N4O2 | +++ |
| 923 | Temocapril HCl | 110221-44-8 | 513.07 | C23H28N2O5S2•HCl | ++ |
| 924 | Temsirolimus (CCl- | 162635-04-3 | 1030.29 | C56H87NO16 | +++++ |
| | 779, NSC 683864) | | | | |
| 925 | Teniposide | 29767-20-2 | 656.7 | C32H32O13S | ++ |
| 926 | Terbinafine | 91161-71-6 | 291.43 | C21H25N | ++ |
| 927 | Terbinafine HCl | 78628-80-5 | 327.89 | C21H26ClN | ++++ |
| 928 | Terconazole | 67915-31-5 | 532.46 | C26H31Cl2N5O3 | ++ |
| 929 | Terephthalic acid | 100-21-0 | 166.13 | C8H6O4 | ++ |
| 930 | Terfenadine | 50679-08-8 | 471.67 | C32H41NO2 | +++++ |
| 931 | Terpinen-4-ol | 562-74-3 | 154.25 | C10H18O | ++ |
| 932 | Testosterone | 315-37-7 | 400.59 | C26H40O3 | +++++ |
| | Enanthate | | | | |
| 933 | Tetrabenazine | 58-46-8 | 317.42 | C19H27NO3 | +++ |
| 934 | Tetracycline | 64-75-5 | 480.90 | C22H25ClN2O8 | ++ |
| | Hydrochloride | | | | |
| 935 | tetrahydro-L- | 69056-38-8 | 314.17 | C9H17Cl2N5O3 | +++ |
| | Biopterin | | | | |
| | (hydrochloride) | | | | |
| 936 | Tetrandrine | 518-34-3 | 622.76 | C38H42N2O6 | ++ |
| 937 | TG101348 | 936091-26-8 | 524.68 | C27H36N6O3S | ++++ |
| | (SAR302503) | | | | |
| 938 | TH-302 | 918633-87-1 | 449.04 | C9H16Br2N5O4P | ++ |
| 939 | Thiamine HCl | 67-03-8 | 337.3 | C12H18Cl2N4OS | ++ |
| | (Vitamin B1) | | | | |
| 940 | Thioridazine HCl | 130-61-0 | 407 | C21H26N2S2•HCl | ++++ |
| 941 | Thiostrepton | 1393-48-2 | 1664.89 | C72H85N19O18S5 | ++ |
| 942 | Thonzonium Bromide | 553-08-2 | 591.71 | C32H55BrN4O | +++ |
| 943 | Thymidine | 50-89-5 | 242.2 | C10H14N2O5 | ++ |
| 944 | Thymol | 89-83-8 | 150.22 | C10H14O | + |
| 945 | Ticagrelor | 274693-27-5 | 522.57 | C23H28F2N6O4S | ++ |
| 946 | Tideglusib | 865854-05-3 | 334.39 | C19H14N2O2S | ++ |
| 947 | Tioconazole | 65899-73-2 | 387.71 | C16H13Cl3N2OS | +++ |
| 948 | Tirapazamine | 27314-97-2 | 178.15 | C7H6N4O2 | ++++ |
| 949 | Tiratricol | 51-24-1 | 621.93 | C14H9I3O4 | +++ |
| 950 | Tivantinib (ARQ 197) | 905854-02-6 | 369.42 | C23H19N3O2 | ++ |
| 951 | Tivozanib (AV-951) | 475108-18-0 | 454.86 | C22H19ClN4O5 | +++ |
| 952 | TOK-001 | 851983-85-2 | 388.56 | C26H32N2O | ++ |
| 953 | Tolazamide | 1156-19-0 | 311.4 | C14H21N3O3S | ++++ |
| 954 | Tolcapone | 134308-13-7 | 273.24 | C14H11NO5 | ++++ |
| 955 | Tolfenamic Acid | 13710-19-5 | 261.7 | C14H12ClNO2 | ++ |
| 956 | Tolnaftate | 2398-96-1 | 307.41 | C19H17NOS | ++++ |
| 957 | Toltrazuril | 69004-03-1 | 425.38 | C18H14F3N3O4S | ++++ |
| 958 | Topiroxostat | 577778-58-6 | 248.24 | C13H8N6 | +++ |
| 959 | Toremifene Citrate | 89778-27-8 | 598.08 | C32H36ClNO8 | +++ |
| 960 | Torsemide | 56211-40-6 | 348.42 | C16H20N4O3S | ++++ |
| 961 | Tramiprosate | 3687-18-1 | 139.17 | C3H9NO3S | +++++ |
| 962 | Tranilast | 53902-12-8 | 327.33 | C18H17NO5 | +++ |
| 963 | Trans-Anethole | 4180-23-8 | 148.2 | C10H12O | ++ |
| 964 | Tranylcypromine | 1986-47-6 | 169.65 | C9H11N•HCl | ++++ |
| | hydrochloride | | | | |
| 965 | Trehalose (hydrate) | 6138-23-4 | 378.33 | C12H22O11•2H2O | ++++ |
| 966 | Tretinoin (Aberela) | 302-79-4 | 300.4 | C20H28O2 | +++ |
| 967 | Triacetonamine | 826-36-8 | 155.24 | C9H17NO | ++ |
| 968 | Triclabendazole | 68786-66-3 | 359.66 | C14 H9Cl3N2OS | +++ |
| 969 | Triclosan | 3380-34-5 | 289.5 | C12H7Cl3O2 | +++++ |
| 970 | Trifluoperazine 2HCl | 440-17-5 | 480.42 | C21H24F3N3S•2HCl | ++++ |
| 971 | Triflupromazine | 1098-60-8 | 388.88 | C18H20ClF3N2S | +++++ |
| | hydrochloride | | | | |
| 972 | Triiodothyronine | 6893-02-3 | 650.97 | C15H12I3NO4 | ++ |
| 973 | Trilostane | 13647-35-3 | 329.43 | C20H27NO3 | ++ |
| 974 | Trimethobenzamide | 554-92-7 | 424.92 | C21H29ClN2O5 | ++ |
| | hydrochloride | | | | |
| 975 | Trimipramine | 521-78-8 | 410.5 | C20H26N2•C4H4O4 | ++ |
| | (maleate) | | | | |
| 976 | Troglitazone | 97322-87-7 | 441.54 | C24H27NO5S | +++ |
| 977 | Tropine | 120-29-6 | 141.21 | C8H15NO | ++ |
| 978 | Tryptamine | 61-54-1 | 160.22 | C10H12N2 | ++ |
| 979 | TSU-68 | 252916-29-3 | 310.35 | C18H18N2O3 | ++ |
| | (SU6668, Orantinib) | | | | |
| 980 | Tucidinostat | 1616493-44-7 | 390.41 | C22H19FN4O2 | ++ |
| 981 | Tyramine | 51-67-2 | 137.18 | C8H11NO | ++ |
| 982 | Udenafil | 268203-93-6 | 516.66 | C25H36N6O4S | ++ |
| 983 | UK 14,304 | 59803-98-4 | 292.13 | C11H10BrN5 | ++++ |
| 984 | UMP | 58-97-9 | 324.18 | C9H13N2O9P | ++ |
| 985 | Undecanoic Acid | 112-37-8 | 186.29 | C11H22O2 | +++ |
| 986 | Uric Acid | 69-93-2 | 168.11 | C5H4N4O3 | + |
| 987 | Uridine triacetate | 4105-38-8 | 370.31 | C15H18N2O9 | ++ |
| 988 | Ursodeoxycholic Acid | 128-13-2 | 392.6 | C24H40O4 | + |
| 989 | Ursolic acid | 77-52-1 | 456.68 | C30H48O3 | +++++ |
| 990 | UTP, Trisodium Salt | 19817-92-6 | 550.09 | C9H12N2Na3O15P3 | ++ |
| 991 | Vadadustat | 1000025-07-9 | 306.7 | C14H11ClN2O4 | ++++ |
| 992 | Valbenazine tosylate | 1639208-54-0 | 762.97 | C38H54N2O10S2 | ++ |
| 993 | Valproic acid | 99-66-1 | 144.21 | C8H16O2 | +++ |
| 994 | Vandetanib (ZD6474) | 443913-73-3 | 475.35 | C22H24BrFN4O2 | ++++ |
| 995 | Vanillic acid | 121-34-6 | 168.15 | C8H8O4 | ++ |
| 996 | Vardenafil | 224785-90-4 | 488.6 | C23H32N6O4S | ++++ |
| 997 | Vatalanib (PTK787) | 212141-51-0 | 419.73 | C20H15ClN4•2HCl | +++ |
| | 2HCl | | | | |
| 998 | Venlafaxine | 99300-78-4 | 313.86 | C17H27NO2•HCl | ++ |
| 999 | Verteporfin | 129497-78-5 | 718.79 | C41H42N4O8 | +++++ |
| 1000 | Vidofludimus | 717824-30-1 | 355.36 | C20H18FNO4 | ++ |
| 1001 | Vilanterol trifenatate | 503070-58-4 | 774.77 | C44H49Cl2NO7 | +++ |
| 1002 | Vinblastine sulfate | 143-67-9 | 909.05 | C46H60N4O13S | ++ |
| 1003 | Vincamine | 1617-90-9 | 354.44 | C21H26N2O3 | ++ |
| 1004 | Vincristine sulfate | 2068-78-2 | 923.04 | C46H58N4O14S | +++ |
| 1005 | Vinorelbine | 71486-22-1 | 778.93 | C45H54N4O8 | ++ |
| 1006 | Vinpocetine | 42971-09-5 | 350.45 | C22H26N2O2 | +++ |
| 1007 | Vitamin A | 68-26-8 | 286.45 | C20H30O | +++ |
| 1008 | Vitamin D2 | 50-14-6 | 396.65 | C28H44O | +++ |
| | (Ergocalciferol) | | | | |
| 1009 | Vitamin D3 | 67-97-0 | 384.64 | C27H44O | +++ |
| 1010 | Vitamin K1 | 84-80-0 | 450.7 | C31H46O2 | ++ |
| 1011 | Vortioxetine | 508233-74-7 | 298.45 | C18H22N2S | +++++ |
| 1012 | Vortioxetine (Lu | 960203-27-4 | 379.36 | C18H22N2S•HBr | +++++ |
| | AA21004) HBr | | | | |
| 1013 | VRT752271 | 869886-67-9 | 433.33 | C21H22Cl2N4O2 | ++++ |
| 1014 | VX-222 (VCH-222, | 1026785-59-0 | 445.6 | C25H35NO4S | ++ |
| | Lomibuvir) | | | | |
| 1015 | VX-661 | 1152311-62-0 | 520.5 | C26H27F3N2O6 | ++ |
| 1016 | VX-745 | 209410-46-8 | 436.27 | C19H9Cl2F2N3OS | ++ |
| 1017 | VX-809 | 936727-05-8 | 452.41 | C24H18F2N2O5 | +++ |
| 1018 | Wedelolactone | 524-12-9 | 314.25 | C16H10O7 | ++ |
| 1019 | WS6 | 1421227-53-3 | 568.59 | C29H31F3N6O3 | +++ |
| 1020 | xanthine | 69-89-6 | 152.11 | C5H4N4O2 | ++ |
| 1021 | Xylose | 25990-60-7 | 150.13 | C5H10O5 | ++++ |
| 1022 | YM155 | 781661-94-7 | 443.3 | C20H19BrN4O3 | +++++ |
| 1023 | Zafirlukast | 107753-78-6 | 575.68 | C31H33N3O6S | +++++ |
| 1024 | Zalcitabine | 7481-89-2 | 211.22 | C9H13N3O3 | ++++ |
| 1025 | Zidovudine | 30516-87-1 | 267.24 | C10H13N5O4 | +++ |
| 1026 | Zinc Pyrithione | 13463-41-7 | 317.7 | C10H8N2O2S2Zn | +++++ |
| 1027 | Zingerone | 122-48-5 | 194.23 | C11H14O3 | ++ |
| 1028 | ZIP | 863987-12-6 | 1928.4 | C90H154N30O17 | ++++ |
| 1029 | Ziprasidone HCl | 122883-93-6 | 449.4 | C21H21ClN4OS•HCl | ++ |

TABLE 15
Functional Oil Myofibroblast Cytotoxicity Activity

| Cmpd No. | Chemical Name | CAS No. | Activity |
|---|---|---|---|
| 1030 | Aloe Vera Inner Leaf Gel | 94349-62-9 | + |
| 1031 | Calendula Oil (CDA) | 70892-20-5 | + |
| 1032 | Copaiba Oil | 8001-61-4 | + |
| 1033 | Elemi Oil (EMO) | 8023-89-0 | + |
| 1034 | Ho Wood Oil (HWO) | 8022-91-1 | ++ |
| 1035 | Niaouli Oil | — | + |
| 1036 | Patchouli Oil | 8014-09-3 | ++ |
| 1037 | Vetiver Essential Oil | — | +++ |
| 1038 | Vetiver Oil Haiti | — | +++ |
| 1039 | Wintergreen | — | + |
| 1040 | Ylang Ylang Oil | 83863-30-3 | ++ |
| 1041 | Ajowan | 8001-99-8 | ++ |
| 1042 | Argan Nut Oil (AGN) | 223747-87-3 | + |
| 1043 | Beet Juice Powder | 7659-95-2 | ++ |
| 1044 | Black Cumin Seed Oil | 8014-13-9 | ++ |
| 1045 | Camellia Oil (CMA) | 68916-73-4 | + |
| 1046 | Cassia | 8007-80-5 | ++ |
| 1047 | Cedarwood essential Oil | 8000-27-9 | ++ |
| 1048 | Chamomile German Oil (CGB) | 8002-66-2 | ++ |
| 1049 | Coriander | 8008-52-4 | + |
| 1050 | Eucalyptus Mountain Herbs | 8000-48-4 | + |
| 1051 | Fir Needle | 8021-29-2 | + |
| 1052 | Frankincense | 8016-36-2 | ++ |
| 1053 | Geranium Egyptian | — | + |
| 1054 | Geranium Oil | — | + |
| 1055 | Helichrysum | 90045-56-0 | ++ |
| 1056 | Hyssop Oil | 8006-83-5 | ++ |
| 1057 | Jasmine Oil | 8022-96-6 | ++ |
| 1058 | Juniper Berry | 8002-68-4 | + |
| 1059 | Kanuka Oil (Kunzea) | — | ++ |
| 1060 | Lavandin Oil (Lavandula Hybrida | 8022-15-9 | ++ |
| | Grosso) | | |
| 1061 | Lavender | 8000-28-0 | + |
| 1062 | Litsea Cubeba Oil (LCO) | 68855-99-2 | ++ |
| 1063 | Michelia Alba Leaf | 8006-76-6 | + |
| 1064 | Nerolina Oil | 8014-68-4 | ++ |
| 1065 | Palmarosa Oil | 8014-19-5 | ++ |
| 1066 | Palo Santo | 959130-05-3 | + |
| 1067 | Patchouli Oil Dark | — | ++ |
| 1068 | Patchouli Oil Light | — | ++ |
| 1069 | Patitgrain (Sicily) | 8014-17-3 | + |
| 1070 | Peppermint Japanese | 68917-18-0 | ++ |
| 1071 | Pine Scotch | 8023-99-2 | + |
| 1072 | Ravensara Wild | 91770-56-8 | + |
| 1073 | Roman Chamomile Oil | — | + |
| 1074 | Rosemary Oil (RMO) | 8000-25-7 | ++ |
| 1075 | Sage Dalmatian | 8022-56-8 | ++ |
| 1076 | Sea Buckthorn Fruit Oil | 225234-03-7 | + |
| 1077 | Spearmint | 8008-79-5 | + |
| 1078 | Star Anise | 84650-59-9 | + |
| 1079 | Sweet Almond Oil | 8007-69-0 | ++ |
| 1080 | Tamanu Oil | 241148-25-4 | ++ |
| 1081 | Thyme Red | — | +++ |
| 1082 | Thyme White | — | + |
| 1083 | Vanzan | 11138-66-2 | + |
| 1084 | Winter Savory | 90106-57-3 | +++ |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of treating, reducing or ameliorating fibrotic diseases and disorders in a subject, comprising administration of comprising α-pinene, aloe vera inner leaf gel, and one or more adjuvants to a subject in need thereof in combination with the administration of Xiaflex.

2. The method of claim 1 wherein the manganese-platinum organic complex is one or more of MNTP-Bio2 and MNT4MPYP.

3. The method of claim 1 wherein the adjuvant is one or more of 4-(nicotinamido)butanic acid, 5,7-dihydroxyflavanone, allicin, allopurinol, allopurinol sodium, alpha tocopherol, ammonium purrolidyldithiocarbamate, anisodamine, arachidonic acid, asarinin (desmin), ascorbic acid (vitamin C), ascorbyl palmitate, bemethyl, beta-carotene, bilirubin, bromovanillin, butylated hydroxytoluene, butyric acid, caffeic acid, calcium ascorbate, carvacrol, chlorovanillin, Chromal, chromanol, cianidanol (catechin), cyanidin, cyanidin-3-glucose, diallyl sulfide, dihydroferulic acid, dihydrolipoic acid, dimethyl sulfoxide (DMSO), DL-alpha-tocopherol acetate, epicatechin, erythorbic acid, eugenol, ferulic acid, glutathione, glycylglycine, hydroxxytyrosol, iso-ascorbic acid, kaempferol, lecithin (hydrogenated), α-lipoic acid, lithospermate b, lithospermic acid, lithospermic acid b, luteolin, lycopene, manganese-platinum organic complexes (e.g., MntP-bio2 and Mnt4MPyP), melatonin, morin, morin hydrate, nicareven, pegorgotein, pentoxifyline, phytic acid, pinocembrin, proamipider, probucol, probucolc (Lorelco™), propyl gallate, puerarin, quercitin, rebamipide, rel-alpha (vitamin E), reservatrol, retinol, rosemarinate, rosmarinic acid, salvianolic acid a, sesamol, silibyn (silininin), silymarin, sodium ascorbate, sodium erythrobate, sodium ferulate, sodium terephthalate, S.R.R alpha-tocopherol, terephthalic acid, tetrahydro-curcumin, thicotic acid, tocopheryl acetate, trans-caffeic acid, Trimidox (vetoquinol), Trocotrienl, Trolox, Tyrosol, ubiquinol-10, ubiquinone, uric acid, vanillin, verbascoside, violuric acid, vitamin A, vitamin E, vitamin E acetate.

4. The method of claims 1-3 wherein the adjuvant is one or more of 1-tetradecanol, acai berry, agave, alanine, anethole anise camphor, apricot kernal oil, argan nut oil, arginine, soluble collagen, arctic fish (AF) collagen, baobab oil, bees wax, benzocaine, benzoin, benzyl alcohol, black cumin seed, cabreuva oil, caffeic acid, calendula oil, camellia oil, caprylic, capric triglyceride, carvacrol, carvone oil, caryophyllene, cedarwood essential oil, cetyl alcohol, chamomile german oil, copaiba oil, elemi oil, ferulic acid, geranium oil, Ghana shea butter, glutamine, glycerol, glycine, histidine, ho wood oil, hydroxyproline, isoleucine, jasmine oil, kaempferol, kanuka oil, lavandin oil, lecithin, leucine, limonene oil, linoleic acid, litsea cubeba oil, lysine, marula oil, methyl sulfonyl methane (MSM), myrrh, nerolina oil, niacinamide, niaouli oil, oleic acid, palmarosa oil, palo santo, panthenol, peppermint, praline, protocatechuic acid, rose hip oil, rosemary oil, sacha inchi oil, sea buckthorn oil, sericin, shea butter, sodium polyacrylate, tamanu oil, terpineol, thyme red, white, vetiver, vitamin A, vitamin B3 (Niacin), vitamin C, winter savory.

5. The method of claims 1-4, wherein the composition further comprises one or more of from about 1.0 to about 5.0 wt. % α-pinene, from about 0.1 to about 1.0 wt. % 1-tetradecanol, from about 1.0 to about 5.0 wt. % collagen, from about 0.1 to about 3.0 wt. % agave nectar, from about 0.1 to about 2.0 wt. % alanine, from about 10.0 to about 20.0 wt. % aloe vera inner leaf gel, from about 0.1 to about 2.0 wt. % apricot kernal oil, from about 0.1 to about 2.0 wt. % argan nut oil, from about 0.1 to about 2.0 wt. % arginine, from about 0.1 to about 1.0 wt. % baobab oil, from about 1.0 to about 5.0 wt. % bees wax, from about 0.1 to about 2.0 wt. % benzoin, from about 1.0 to about 5.0 wt. % benzyl alcohol, from about 1.0 to about 5.0 wt. % black cumin seed oil, from about 0.1 to about 3.0 wt. % calendula oil, from about 0.1 to about 3.0 wt. % camellia oil, from about 0.1 to about 3.0 wt. % caprylic, capric triglyceride, from about 0.1 to about 3.0 wt. % carvacrol, from about 0.1 to about 3.0 wt. % caryophyllene, from about 1.0 to about 5.0 wt. % cetyl alcohol, from about 0.1 to about 3.0 wt. % chamomile german oil, from about 0.1 to about 3.0 wt. % copaiba oil, from about 20 to about 35.0 wt. % distilled water, from about 0.1 to about 3.0 wt. % elemi oil, from about 0.1 to about 3.0 wt. % ferulic acid, from about 0.1 to about 1.0 wt. % geranium oil, from about 10.0 to about 20.0 wt. % shea butter, from about 0.1 to about 3.0 wt. % glutamine, from about 0.1 to about 3.0 wt. % glycerol, from about 0.1 to about 2.0 wt. % glycine, from about 0.1 to about 2.0 wt. % histidine, from about 0.1 to about 1.0 wt. % ho wood oil, from about 0.1 to about 2.0 wt. % hydroxyproline, from about 0.1 to about 2.0 wt. % isoleucine, from about 0.1 to about 1.0 wt. % jasmine oil, from about 0.1 to about 1.0 wt. % kanuka oil, from about 0.1 to about 3.0 wt. % lavandin oil, from about 0.5 to about 4.0 wt. % lecithin, from about 0.1 to about 2.0 wt. % leucine, from about 1.0 to about 5.0 wt. % limonene oil, from about 0.1 to about 2.0 wt. % linoleic acid, from about 0.1 to about 2.0 wt. % litsea cubeba oil, from about 0.1 to about 2.0 wt. % lysine, from about 0.1 to about 1.0 wt. % marula oil, from about 0.5 to about 3.0 wt. % methyl sulfonyl methane, from about 0.1 to about 1.0 wt. % nerolina oil, from about 0.1 to about 1.0 wt. % niacinamide, from about 0.1 to about 2.0 wt. % oleic acid, from about 0.1 to about 1.0 wt. % palmarosa oil, from about 0.1 to about 3.0 wt. % panthenol, from about 0.1 to about 2.0 wt. % praline, from about 0.1 to about 3.0 wt. % protocatechuic acid, from about 0.1 to about 2.0 wt. % rosemary oil, from about 0.1 to about 2.0 wt. % sea buckthorn oil, from about 1.0 to about 5.0 wt. % sericin, from about 0.1 to about 3.0 wt. % sodium polyacrylate, from about 0.1 to about 2.0 wt. % tamanu oil, from about 0.1 to about 1.0 wt. % vitamin A, from about 0.1 to about 3.0 wt. % Vitamin B3 (niacin), from about 0.1 to about 1.0 wt. % vitamin C, and from about 0.1 to about 1.0 wt. % vitamin E.

6. The method of claim 1-5, wherein the administration of the composition is topical administration.

7. The method of claim 6, wherein the administration of the composition is topical administration to skin.

8. A method of treating, reducing or ameliorating fibrotic diseases and disorders in a subject, the method comprising the step of contacting one or more cells of said subject with a therapeutically effective amount of the composition of one of claims 1 to 7 in combination with the administration of Xiaflex.
